(19) United States
(12) Patent Application Publication
(10) Pub. No.: US 2003/0070184 A1
(43) Pub. Date: Apr. 10, 2003

(54) LIGANDS FOR DISCOIDIN DOMAIN RECEPTOR TYROSINE KINASES AND COMPLEXES THEREOF

(76) Inventors: WOLFGANG VOGEL, TORONTO (CA); ANTHONY PAWSON, TORONTO (CA)

Correspondence Address: MERCHANT & GOULD PC, P.O. BOX 2903, MINNEAPOLIS, MN, 55402-0903, US

(21) Appl. No.: 09/355,815

(22) PCT Filed: Feb. 5, 1998
(86) PCT No.: PCT/CA98/00093

(51) Int. Cl. A01K 67/00; C07H 21/04; C12N 9/12; C12P 21/02; C12N 5/06; C07K 14/705

(52) U.S. Cl. 800/8; 435/69.1; 435/194; 435/325; 435/320.1; 530/350; 536/23.5

(57) ABSTRACT

Members of the collagen family are ligands for the discoidin domain receptor tyrosine kinases, DDR1 and DDR2. Collagen directly interacts with the extracellular domains and evokes tyrosine phosphorylation of DDRs in a time and concentration dependent manner. Collagen activation of DDR1 induced phosphorylation of a docking site for the Shc phosphotyrosine binding domain. Therefore, isolated complexes are described comprising (a) a discoidin domain receptor tyrosine kinase or a part thereof, and a collagen or a part thereof; or (b) a discoidin domain receptor tyrosine kinase or a part thereof and Shc or PTB binding domain of Shc or a protein containing a PDZ domain or a PDZ domain. Compositions, methods, and uses are also described based on the interaction of DDRs with collagens.

### FIELD OF THE INVENTION

The invention relates to novel complexes, methods of activating a discoidin domain receptor tyrosine kinase (DDR)-mediated signaling pathway in a cell, and methods of identifying substances that affect the pathway.

### BACKGROUND OF THE INVENTION

The cloning of cDNAs for mammalian receptor tyrosine kinases (RTK) has recently resulted in the identification of a new subfamily of receptors which possess an extracellular domain related to the lectin discoidin, found in the slime mould Dictyostelium dscoideum. Two distinct RTKs with discoidin repeats have been identified, namely discoidin domain receptor 1 (DDR1)(also termed DDR, MCK10, Cak, NEP, trkE, Ptk3 or RTK6) (1-7) and discoidin domain receptor 2 (DDR2) (also termed CCK2, TKT or Tyro 10) (1, 8, 9). DDR1 is primarily expressed in epithelial cells (1), and is particularly abundant in neuroepithelial cells during mouse embryonic development (4). High levels of DDR1 have been detected in human ovarian and breast cancer samples, suggesting that DDR1 overexpression may be involved in tumor development (1, 10). The extracellular domain of DDR1 possesses the motif RXRR, which is a potential recognition site for endoprotease cleavage. Indeed a considerable fraction of DDR1 is processed to a truncated membrane-associated β-subunit and a soluble α-subunit (1).

In comparison with other receptor tyrosine kinases, DDR1 has a relatively long juxtamembrane region, which is modified by alternative splicing to yield two distinct DDR1 isoforms (1). The DDR1a isoform has 139 amino acids in the juxtamembrane region, whereas the DDR1b isoform differs from the a-isoform by the incorporation of an additional stretch of 37 amino acids in the juxtamembrane region, encoded by an extra exon (7).

DDR receptors may be involved in tumorigenesis. Both a- and b-specific DDR1 RNA transcripts have been detected in various human ovarian (7) and breast² cancer cell lines. In situ analysis of several human primary mammary carcinomas has shown that the expression of DDR1 mRNA can be at least 3-fold higher in tumor cells tan in the adjacent normal epithelia (Barker et al., Oncogene 11: 569-575, 1995). Using probes for both genes, in situ hybridization on adjacent sections of human ovary or lung carcinomas has shown that DDR1 is expressed in the tumor cells themselves, whereas DDR2 is detected in the stromal cells surrounding the tumor (Alves et al., Oncogene 10: 609-618, 1995).

The b-specific insert in DDR1b displays sequence motifs which suggests that the insert may be involved in signaling downstream of the DDR1 receptor (1). Notably, the b-isoform-specific insert contains the sequence LLSNPAY, which potentially might serve as a docking site for the phosphotyrosine-binding (PTB) domain of the Shc adaptor protein.

The Shc protein contains both a C-terminal SH2 domain and an N-terminal PTB domain of approximately 160 residues, that, unlike SH2 domains, recognizes phosphotyrosine (pTyr) sites with the consensus sequence: hydrophobic-X-Asn-Pro-X-pTyr (11-14). Such phosphorylated motifs can be found in the juxtamembrane region of the nerve growth factor (NGF) receptor, and in the C-terminal tails of the epidermal growth factor (EGF) receptor, ErbB2 and ErbB3 (15-18). Interaction of the Shc PTB domain with activated receptors can stimulate Shc phosphorylation at Tyr 317, within a motif (YVNV) which is recognized by the Grb2 SH2 domain (19). The association of phosphorylated Shc with Grb2 provides a mechanism by which Shc can stimulate the Ras pathway. In addition to growth factor receptors, cytoplasmic proteins such as the polyomavirus middle T antigen and the SHIP SH2-containing inositol phosphatase contain NPXY motifs which can potentially bind the Shc PTB domain upon phosphorylation (15, 20).

Analysis of both the Shc PTB domain and its binding sites has shown that an Asn three residues N-terminal to the phosphorylated Tyr (at the −3 position) is essential for binding, and that Pro is favored at the −2 position (21-25). These residues are important for forming a β-turn that positions the phosphotyrosine in a basic pocket of the PTB domain (26). High affinity binding of phosphopeptides to the Shc PTB domain also requires a hydrophobic residue at the −5 position (16, 18). In addition, a hydrophobic residue at the −6 position can make contact with the PTB domain (26). The LLSNPAY sequence within the b-isoform-specific insert of DDR1 conforms to this consensus for Shc PTB-binding.

### SUMMARY OF THE INVENTION

The present inventors have significantly shown that members of the collagen family are ligands for the discoidin domain receptor tyrosine kinases, DDR1 (also known as MCK-10, DDR, NEP, cak, trkE, RTK6, and ptk3) and DDR2 (also known as CCK-2, tyro-10, and TKT). Collagen was found to directly interact with the extracellular domains and evoke tyrosine phosphorylation of DDRs in a time and concentration dependent manner. In particular, collagen types I, II, III, IV, and V were shown to be good ligands for DDR1. Collagen type I and III were shown to be highly potent ligands for DDR2; while collagen type II and V showed moderate activity. The present inventors also showed that the glycosylation of collagen is essential for DDRs activation, in particular DDR2 activation. Stimulation of DDR receptor tyrosine kinase activity required the native triple helical structure of collagen.

Collagen activation of DDR1 induced phosphorylation of a docking site for the Shc phosphotyrosine binding domain, whose presence is controlled by alternative splicing. In particular, the present inventors showed by direct evidence that the PTB domain of the Shc adaptor protein binds selectively to DDR1b utilizing the LLSNPAY motif encoded by the alternatively spliced b-specific exon. Activation of DDR2 by collagen was also shown to result in the up-regulation of matrix metalloproteinase-1 (MMP-1) expression. Thus, DDR1 and DDR2 are novel collagen receptors that can control cellular responses to the extracellular matrix.

Broadly stated the present invention provides an isolated complex comprising a DDR or a part thereof, and a collagen or a part thereof, or a complex comprising a DDR or a part thereof and Shc, or a protein containing a PDZ domain. Peptides derived from the binding domain of a DDR that interacts with a collagen or part of a collagen, or interacts with Shc or a PDZ domain, or a molecule derived from the binding domain of collagen'that interacts with a DDR or a part thereof are also contemplated. The invention also includes antibodies specific for the complexes and peptides.

The present invention also provides a method of modulating, and in particular activating, a discoidin domain receptor tyrosine kinase (DDR) -mediated signaling pathway in a cell, comprising reacting a discoidin domain receptor tyrosine kinase protein, or an isoform or a part of the protein on the cell, with a collagen or part of a collagen, thereby modulating the signaling pathway in the cell. In an embodiment, the protein or part of the protein comprises an oligomerized receptor or the extracellular domain or an oligomerized extracellular domain of discoidin domain receptor tyrosine kinase. The pathway may also be activated by employing a complex or peptide of the invention

In an embodiment, the invention contemplates a method for modulating extracellular matrix synthesis, degradation or remodeling responses in a cell comprising reacting a discoidin domain receptor tyrosine kinase protein, or an isoform or a part of the protein having at least 20 contiguous amino acids of the protein on a cell, with a collagen or part of a collagen, thereby modulating extracellular matrix synthesis, degradation or remodelling responses. Extracellular matrix synthesis, degradation, or remodelling responses may be modulated using a complex or peptide of the invention.

Still further the invention provides a method for evaluating a compound for its ability to modulate a DDR-mediated signaling pathway. For example, a substance which inhibits or enhances the interaction of a DDR and a collagen, or a substance which binds to DDR or a part thereof, or to collagen or part of a collagen may be evaluated.

In an embodiment, the invention provides a method for identifying a substance which affects a DDR receptor tyrosine kinase-mediated signaling pathway, comprising the steps of:

(a) reacting a collagen, and at least one discoidin domain receptor tyrosine kinase protein, or an isoform or a part of the protein, and a test substance, wherein the collagen and discoidin domain receptor tyrosine kinase protein are selected so that they bind to form a collagen-discoidin domain receptor tyrosine kinase protein complex; and

(b) comparing to a control in the absence of the substance to determine the effect of the substance.

In particular, a method is provided for identifying a substance which affects a DDR receptor tyrosine kinase-mediated signaling pathway in a cell, comprising

(a) reacting a collagen or part thereof, and at least one discoidin domain receptor tyrosine kinase protein, or an isoform or a part of the protein, and a test substance, wherein the collagen and discoidin domain receptor tyrosine kinase protein are selected so that they bind to form a collagen-discoidin domain receptor tyrosine kinase protein complex, under conditions which permit the formation of collagen-discoidin domain receptor tyrosine kinase protein complexes, and

(b) assaying for complexes, for free substance, for non-complexed collagen, or for activation of the protein.

In an embodiment of the method, the substance is a carbohydrate moiety of a collagen, or a mimetic thereof, or a peptide derived from the domain of a DDR that binds to a collagen, or a mimetic thereof.

The invention still further provides a method for treating or preventing a condition involving a discoidin domain receptor tyrosine kinase-mediated signaling pathway, which method comprises administering to a patient in need thereof an amount of a substance which is effective to interfere with the signaling pathway wherein the substance is (a) a discoidin domain receptor tyrosine kinase or part thereof; (b) a collagen or part thereof; (c) a substance first identified by

(i) reacting a collagen, and at least one discoidin domain receptor tyrosine kinase protein, or an isoform or a part of the protein, and the test substance, wherein the collagen and discoidin domain receptor tyrosine kinase protein are selected so that they bind to form a collagen-discoidin domain receptor tyrosine kinase protein complex; and

(ii) comparing to a control in the absence of the substance to determine the effect of the substance.

The substance may also be an isolated complex comprising a DDR and a collagen; peptides derived from the binding domain of a DDR that interacts with a collagen or part of a collagen, or that interacts with Shc or a PDZ domain; a molecule derived from the binding domain of collagen that interacts with a DDR or a part thereof; or, antibodies specific for the complexes and peptides.

The invention also relates to a pharmaceutical composition which comprises a purified and isolated discoidin domain subfamily receptor tyrosine kinase protein or an isoform or a part of the protein, a collagen or a part of a collagen, a complex, antibody, a peptide of the invention, or a substance as described herein, in an amount effective to affect a discoidin domain receptor tyrosine kinase-mediated signaling pathway, and a pharmaceutically acceptable carrier, diluent or excipient. The composition may comprise an extracellular domain of a discoidin domain receptor tyrosine kinase, or the portion of the extracellular domain which binds to the carbohydrate moiety of a collagen, or mimetics thereof. In an embodiment the composition comprises a collagen or a portion thereof, preferably a carbohydrate moiety of collagen.

The methods and compositions of the invention may be used to alter transformation or metastasis in a mammal, to treat conditions involving structural or functional deregulation of collagens such as Cleidocranial displasia and Sickler syndrome, conditions that require modulation of extracellular matrix synthesis, degradation or remodeling, or to treat conditions requiring modulation of MMP-1 expression (e.g. for use in wound healing).

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1A is an immunoblot of immunoprecipitates with anti-Shc antibodies from cell lysates of human embryonic kidney fibroblast 293 cells transfected with expression plasmids encoding DDR1a (MCK10a) or DDR1b (MCK10b) and stimulated with orthovanadate probed with antibodies to phosphotyrosine;

FIG. 1B is the immunoblot in FIG. 1A reprobed with antibodies against DDR1;

FIG. 1C is the immunoblot in FIG. 1B reprobed with antibodies against Shc;

FIG. 1D is an immunoblot of the total cell lysates used in FIG. 1A analysed by Western blotting with anti-phosphotyrosine antibodies;

FIG. 1E is an immunoblot of proteins from lysates (from 293 cells which had been transfected with DDR1a or DDR1b and stimulated with orthovanadate) that bound to GST-fusion proteins containing the Shc SH2 domain or Shc PTB domain;

FIG. 2A is an immunoblot from an experiment involving incubating GST-Shc PTB domain fusion protein bound to glutathione beads with lysates from DDR1b overexpressing 293 cells in the absence or presence of increasing concentrations of a competing peptide, ALLLSNPApYRLLLA, and detecting bound protein by immunoblotting with antibodies to DDR1;

FIG. 2B a graph representing the analysis of the binding of purified GST-Shc PTB domain to the middle T antigen phosphopeptide by surface plasmon resonance in the presence of increasing amounts of DDR1 phosphopeptide (ALLLSNPApYRLLLA, open circles) or NGF receptor phosphopeptide (HIIENPQpYFSD, closed circles), respectively; where the percentage of Shc PTB domain bound to the chip surface is plotted against the concentration of competing peptides;

FIG. 3A is a two dimensional tryptic phosphopeptide map of the in vivo labeled α-isoform of DDR1;

FIG. 3B is a two dimensional tryptic phosphopeptide map of the in vivo labeled β-isoform of DDR1;

FIG. 3C is a schematic representation of the phosphopeptides in FIGS. 3B and 3F;

FIG. 3D shows the results of the typtic mapping of in vitro labeled protein of the DDR1a isoform;

FIG. 3E shows the results of the tryptic mapping of in vitro labeled protein of the DDR1b isoform;

FIG. 3F shows the results of the tryptic mapping of the DDR1a and DDR1b phosphopeptides;

FIG. 4A is an immunoblot of the results of experiments where DDR1a, DDR1b, a mix of DDR2, and TrkA are transiently expressed in 293 cells stimulated with collagen type I or treated with 100 μM acetic acid, and total cellular lysates are blotted and probed with αpTyr antibodies [Sigma #C-7661: rat tail collagen type I];

FIG. 4B is an immunoblot of the results of experiments where DDR1a, DDR1b, DDR2, and TrkA are trans expressed in 293 cells stimulated with collagen types I or IV, and total cellular lysates are blotted and probed with αpTyr antibodies [Sigma #C3511:bovine skin collagen type I, C-7521: human placenta collagen type IV];

FIG. 4C is an inmmunoblot of the results of experiments where DDR1a, DDR1b, and TrkA are transiently expressed in 293 cells stimulated with type I collagens, and total cellular lysates are blotted and probed with αpTyr antibodies [Sigma #C-8897:rat tail collagen type I, C-7774: human placenta collagen type I];

FIG. 5A is a α-pY blot of total cellular lysates from 293 cells transiently expressing DDR1b in the presence of types I and IV collagens at different concentrations [Sigma #C-7661:rat tail collagen type I, C-0543:mouse collagen type IV);

FIG. 5B is a α-pY blot of total cellular lysates from 293 cells transiently expressing DDR2 stimulated with types I or IV collagen at different concentrations;

FIG. 5C is a a-pY blot of total cellular lysates from 293 cells transiently expressing DDR2 in the presence of type I collagens at different concentrations [CBP: Collaborative Biomedical Products #40236:collagen type I];

FIG. 6 is a α-PY blot of DDR1b immunoprecipitated from lysates of human mammary carcinoma cells stimulated with Collagen I [C-7661], Collagen IV [C-0543] and orthovanadate;

FIG. 7A is a blot of total protein of cell lysates of human kidney fibroblast 293 cells transfected with DDR1b and stimulated with different concentratons of Matrigel, probed with antiphosphotyrosine antibody;

FIG. 7B is the blot in FIG. 7A stripped and reprobed with antibodies raised against DDR1;

FIG. 7C is an antiphosphotyrosine blot of cell lysates of 293 cells transfected with DDR1b and treated with the following reagents: 400 μM acetic acid (a), 50 μl/ml matrigel (b), 10 μg/ml laminin type IV (c), 10 μg/ml fibronectin (d), collagen type IV, partially purified from matrigel by extraction with guanidinium hydrochloride (e) or by extraction with acetic acid and pepsin (f), 10 μg/ml mouse collagen type IV, Sigma C-0543 (g), 10 μg/ml human collagen type IV, Sigma C-5533 (h);

FIG. 7D is an anti-DDR1 antibody blot of cell lysates of 293 cells treated with the following reagents: 400 μM acetic acid (a), 50 μl/ml matrigel (b), 10 μg/ml laminin type IV (c), 10 μg/ml fibronectin (d), collagen type IV, partially purified from matrigel by extraction with guanidinium hydrochloride (e) or by extraction with acetic acid and pepsin (f), 10 μg/ml mouse collagen type IV, Sigma C-0543 (g), 10 μg/ml human collagen type IV, Sigma C-5533 (h);

FIG. 8A is an antiphosphotyrosine antibody blot of cell lysates of 293 cells transfected with plasmids coding for human insulin receptor (Ins-R), DDR1a, DDR1b or DDR2 stimulated with 10 μg/ml mouse collagen type I, 100 nM insulin or left unstimulated;

FIG. 8B is the blot in FIG. 8A reprobed with a mixture of antibodies against DDR1, DDR2 and insulin receptor;

FIG. 9A is an antiphosphotyrosine antibody blot of cellular lysates of 293 cells transfected with DDR1b and stimulated with collagen type I for different periods of time;

FIG. 9B is an antiphosphotyrosine antibody blot of cellular lysates of 293 cells transfected with DDR2 and stimulated with collagen type I for different periods of time;

FIG. 9C is a blot of DDR1 immunoprecipitates from cell lysates of human mammary carcinoma T-47D cells stimulated with collagen Type I for various periods of time

FIG. 9D is the blot of FIG. 9C reprobed with an antibody specific to the C terminus of DDR1;

FIG. 9E is a blot of DDR1 immunoprecipitated from overexpressing 293 cells and subjected to an in vitro kinase reaction;

FIG. 9F a blot of DDR1 immunoprecipitated from T-47D cells and subjected to an in vitro kinase reaction;

FIG. 10A is an antiphosphotyrosine antibody blot of cellular lysates of 293 cells transfected with DDR1b stimulated with 10 μg/ml human collagen types I, III, IV or V and bovine collagen type II.;

FIG. 10B is the blot of FIG. 10A reprobed with receptor specific antibodies for DDR1;

FIG. 10C is an antiphosphotyrosine antibody blot of cellular lysates of 293 cells transfected with DDR2 stimulated with 10 μg/ml human collagen types I, III, IV or V and bovine collagen type II.;

FIG. 10D is the blot of FIG. 10C reprobed with receptor specific antibodies for DDR2;

FIG. 10E is an antiphosphotyrosine antibody blot of DDR1b immunoprecipitated from T-47D cells that had been stimulated for 90 min with collagen types I, II, III, IV, V or gelatin or treated with 1 mM orthovanadate;

FIG. 10F is the blot of FIG. 10E reprobed with DDR1 specific antibody;

FIG. 10G is an antiphosphotyrosine antibody blot of cellular lysates of 293 cells that have been transfected with DDR1b and contacted with human collagen types I, III, IV and V;

FIG. 10H is an antiphosphotyrosine antibody blot of cellular lysates of 293 cells that have been transfected with DDR2 and contacted with human collagen types 1, III, IV and V;

FIG. 11A shows collagen type I isolated from mouse or human tissue or BSA treated with collagenase or pepsin and analyzed by SDS-PAGE and visualized by Coomassie staining;

FIG. 11B is an antiphosphotyrosine antibody blot of cell lysates of 293 cells overexpressing DDR2 stimulated with the collagen type I treated with collagenase or pepsin;

FIG. 11C is the blot in FIG. 11B reprobed with DDR2-specific antibody;

FIG. 11D is a spectrum of mouse collagen type I (500 ng/ml in 10 mM acetic acid) melted in a spectropolarimeter before (squares), and after heat denaturation (diamonds);

FIG. 11E is an antiphosphotyrosine antibody blot of cellular lysates of 293 cells overexpressing DDR2 stimulated with aliquots of mouse collagen type I that has been incubated at various temperatures;

FIG. 12A is a blot of material from lysates of 293 cells overexpressing insulin-receptor, DDR1b or DDR2 in the absence or presence of 50 μg/ml soluble collagen type I, which bound to collagen covalently coupled to agarose;

FIG. 12B is a graph showing the amount of bound ligand from 293 cells transfected with DDR1a (squares), DDR1b (diamonds) or control plasmid (circles) after incubation with various concentrations of iodinated collagen type I;

FIG. 12C is an antiphosphotyrosine antibody blot of cellular lysates from 293 cells overexpressing DDR2 stimulated with collagen type I deglycosylated with sodium m-periodate;

FIG. 13A is a blot of proteins from lysates from 293 cells overexpressing DDR1a or DDR1b which bound to a GST-fusion protein of a PTB domain of Shc detected with an antibody against the C-terminus of DDR1;

FIG. 13B is a graphic representation of the analysis of the binding of purified GST-Shc PTB domain to the middle T antigen phosphopeptide (LSLLSNPTpYSVMRSK) by surface plasmon resonance in the presence of competing amounts of DDR1b phosphopeptide (ALLLSNPApYRLLLA, open circles) or NGF receptor phosphopeptide (HIIENPQpYFSD, closed circles), respectively;

FIG. 14 is a blot with antibodies against MMP-1 of conditioned media from parental and DDR2 overexpressing HT 1080 cells stimulated with collagen type I or TPA for the indicated periods of time;

FIG. 15A is an immunnoblot showing that DDR1a with K618A mutation is no longer activated by collagen;

FIG. 15B is an immunnoblot showing that DDR1a with K618A mutation is no longer activated by collagen;

FIG. 16 is a blot showing that blocking antibodies to α1- or β1-integrins do not inhibit the activation of DDR1;

FIG. 17A is a blot showing that DDR1 is activated by collagen in integrin β1-deficient cells;

FIG. 17B is a blot showing that DDR1 is activated by collagen in integrin β1-deficient cells;

FIG. 18 is a blot that shows that DDR1b activation in integrin β1-deficient cells is as slow as in normal cells, indicating that the protracted activation of DDR1b is not due to the action of integrins;

FIG. 19A is an immunoblot showing that activation of DDR1 and DDR2 receptor does not influence EGF mediated MAPK activation,

FIG. 19B is an immunoblot showing that activation of DDR1 and DDR2 receptor does not influence EGF mediated MAPK activation,

FIG. 19C is an immunoblot showing that activation of DDR1 and DDR2 receptor does not influence EGF mediated MAPK activation;

FIG. 20A shows the nucleotide and amino acid sequence of human DDR1 which is FIG. 1 in Johnson et al, Proc. Natl. Acad. Sci. USA 90: 5677, 1995;

FIG. 20B shows the amino acid sequence of DDR1 from GenBank Accession No. L20817;

FIG. 21 shows an alignment of DDR1a, b, and c sequences where the NPXY motif in the insertion region of DDR1 is underlined with thin and the putative SH3 binding site with solid bars, which is FIG. 1(c) in Alves et al, 1995, Oncogene 10: 609, 1995;

FIG. 22A shows the nucleotide and amino acid sequence of human DDR2 which is GenBank Accession No. X74764; and

FIG. 22B shows the amino acid sequence of human DDR2 from GenBank Accession No. X74764.

### DETAILED DESCRIPTION OF THE INVENTION

Discoidin Domain Receptor Tyrosine Kinase (DDR) and Collagens

The term “discoidin domain receptor tyrosine kinase (DDR)-mediated signaling pathway” used herein refers to the interactions of a discoidin domain receptor tyrosine kinase protein with a collagen or a part thereof, to form a collagen receptor tyrosine kinase protein complex thereby activating a series of downstream regulatory pathways in the cell that affect the cell, for example by controlling gene expression, cell division, cytoskeletal architecture, cell metabolism, migration, cell-cell interactions, spatial positioning, extracellular matrix synthesis and degradation and remodelling, expression of proteins (e.g. up-regulation of MMP-1), and/or cell adhesion. Examples of such downstream regulatory pathways are the GAP/Ras pathway, the pathway that regulates the breakdown of the polyphosphoinositides through phospholipase C (PLC) and the Src/tyrosine kinase and Ras pathways. The pathway includes the interactions of a DDR protein with intracellular signaling molecules including Shc or proteins with PDZ domains.

“Discoidin domain receptor tyrosine kinase (DDR) proteins” refers to a family of receptor tyrosine kinases that contain a discoidin I motif in their extracellular domains. The structure of the extracellular region determines ligand binding specificity. The intracellular regions contain the juxtamembrane and the catalytic kinase domain. Receptor mediated signal transduction is initiated in the receptor expressing cell by ligand binding to the extracellular domain, which facilitates dimerization of the receptor and autophosphorylation.

The hallmarks of a discoidin domain receptor tyrosine kinase are exemplified by the discoidin domain receptor 1 (DDR1) (Di Marco et al, 1993 J. Biol. Chem. 268:24290; Johnson et al, Proc. Natl. Acad. Sci. USA, 90, 5677, 1993; Zerlin et al, 1993 Oncogene 8: 2731; Laval et al., 1994 Cell Growth Differ. 5:1173, Perez et al, 1994, Oncogene 9:211; Sanchez et al., 1994, Proc. Natl. Acad. Sci. USA 91:1819,; Alves, et al, Oncogene 10, 609, 1995; Shelling et al, 1995, Genomics 25:584; Valent et al, 1996, Human Genet. 98:12) and the discoidin domain receptor 2 (DDR2) (Kam et al, 1993, Oncogene 8:3433; Alves, et al, Oncogene 10, 609, 1995 Lai and Lemke et al; 1994). There are three different forms of DDR1, designated a, b, and c, which represent alternative splicing variants of a common primary gene transcript. DDR1 and DDR2 have a high degree of similarity with a match of 78% within the about 150 amino acid-long amino-terminal discoidin I domain. DDR1 contains the consensus sequence RXRR at position 304-307 which represents a possible cleavage signal for the endopeptidase firin. The juxtamembrane domain of the DDR2 receptor comprises 148 amino acids. The DDR1a isoform comprises 139 amino acids in the juxtamembrane region, whereas the b isoform differs from the a isoform by the incorporation of an additional stretch of 37 amino acids in the juxtamembrane region encoded by an extra exon. The b isoform specific motif contains the sequence LLSNPAY which serves as a docking site for the phosphotyrosine-binding (PTB) domain of the Shc adaptor protein.

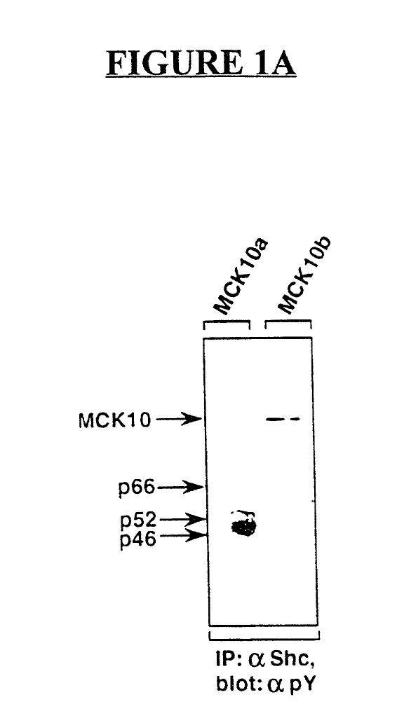

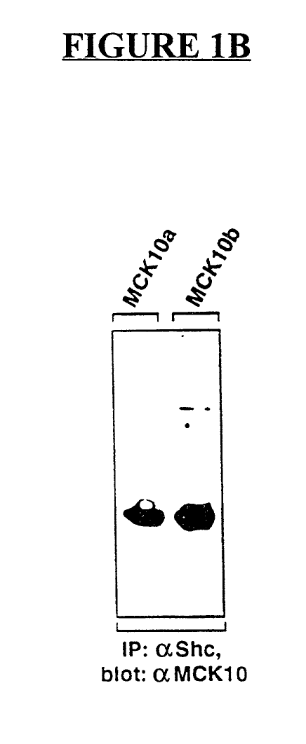

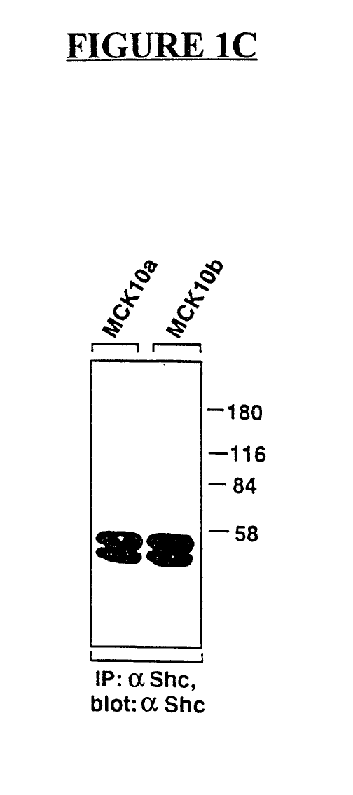

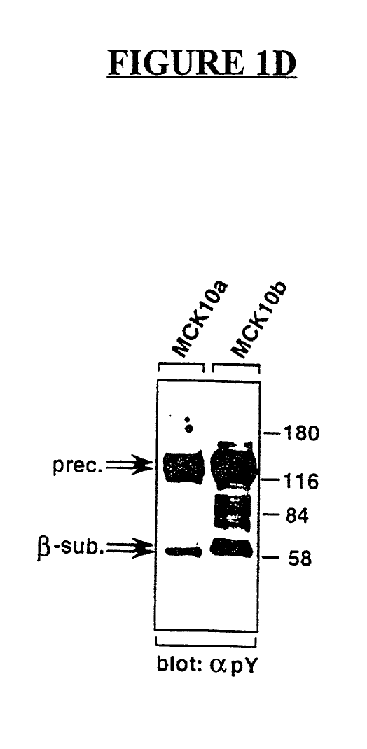

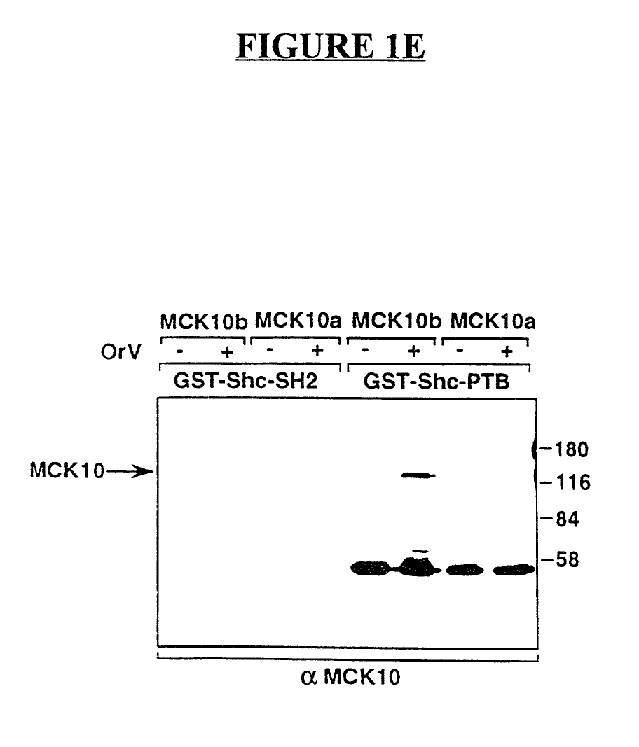

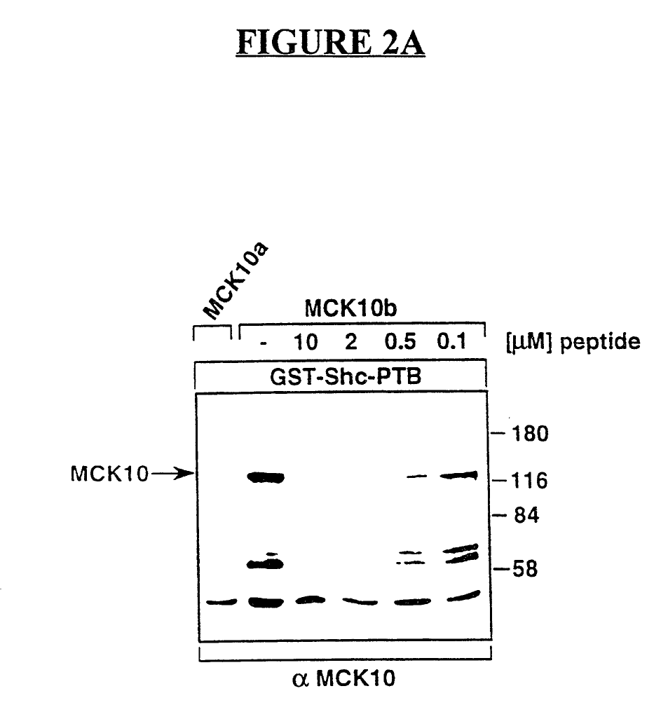

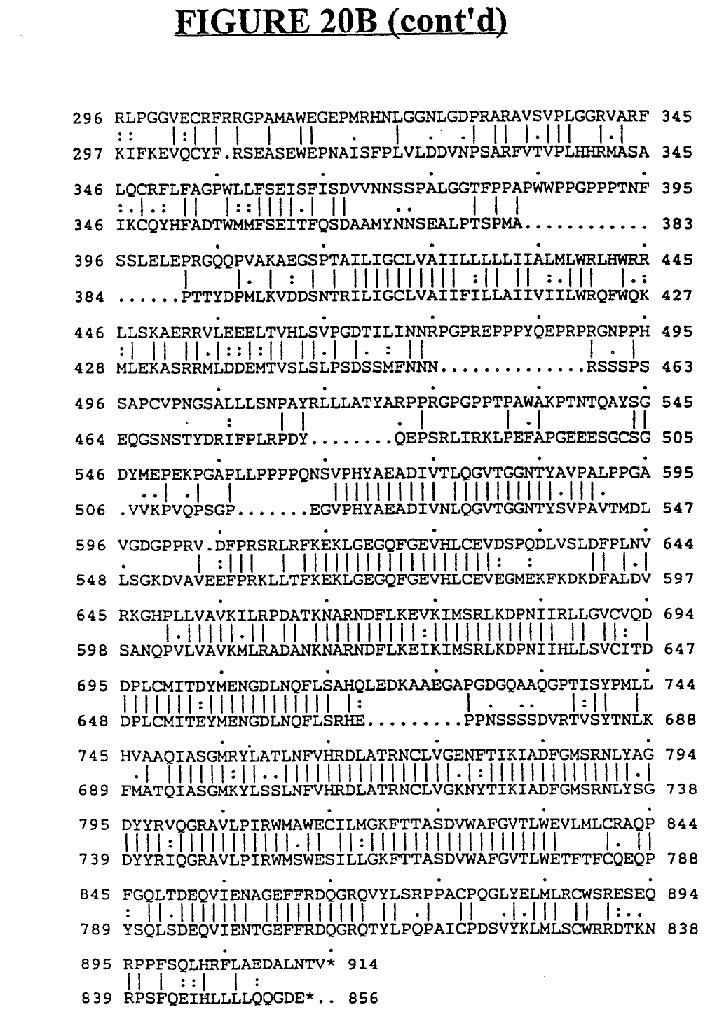

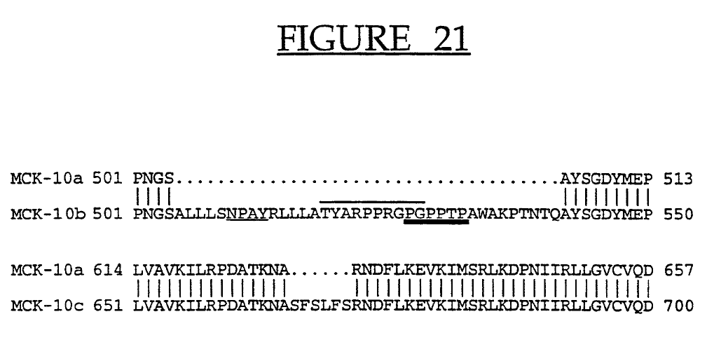

FIGS. 20A and B shows the nucleotide sequence and deduced amino acid sequence of the human DDR1 cDNA (FIG. 1 in Johnson et al, 1993, Supra). The boxed sequence near the N terminus contains the discoidin I-like domain and the box near the C terminus contains the tyrosine kinase domain. The predicted signal peptide and transmembrane domain are underlined; and the proline and glycine residues between the discoidin-I like domain and the tyrosine kinase domain are italicized. The ˆ ˆ symbols underline the most proline and glycine—rich of the connecting region. The juxtamembrane region is between amino acid 468 and amino acid 607. The sequence of DDR1 can also be found in GenBank, Accession Nos. L11315 or L20817. An alignment of the sequences of the spliced DDR1 isoforms is shown in FIG. 21 (FIG. 1 from Alves et al, 1995).

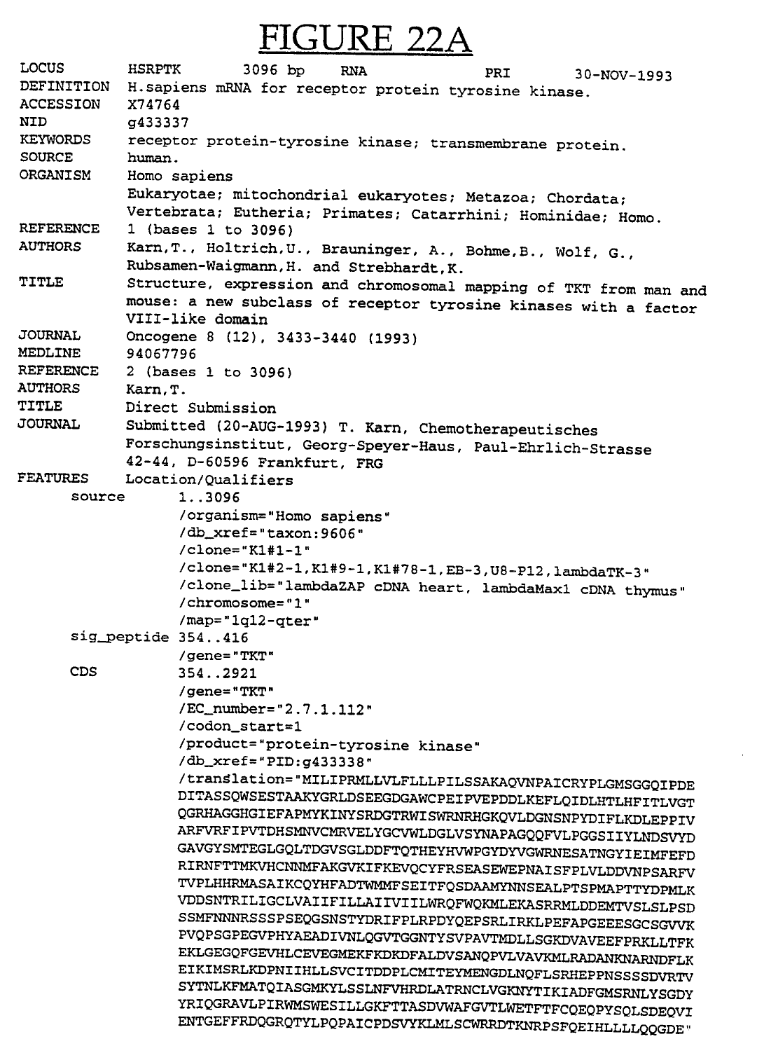

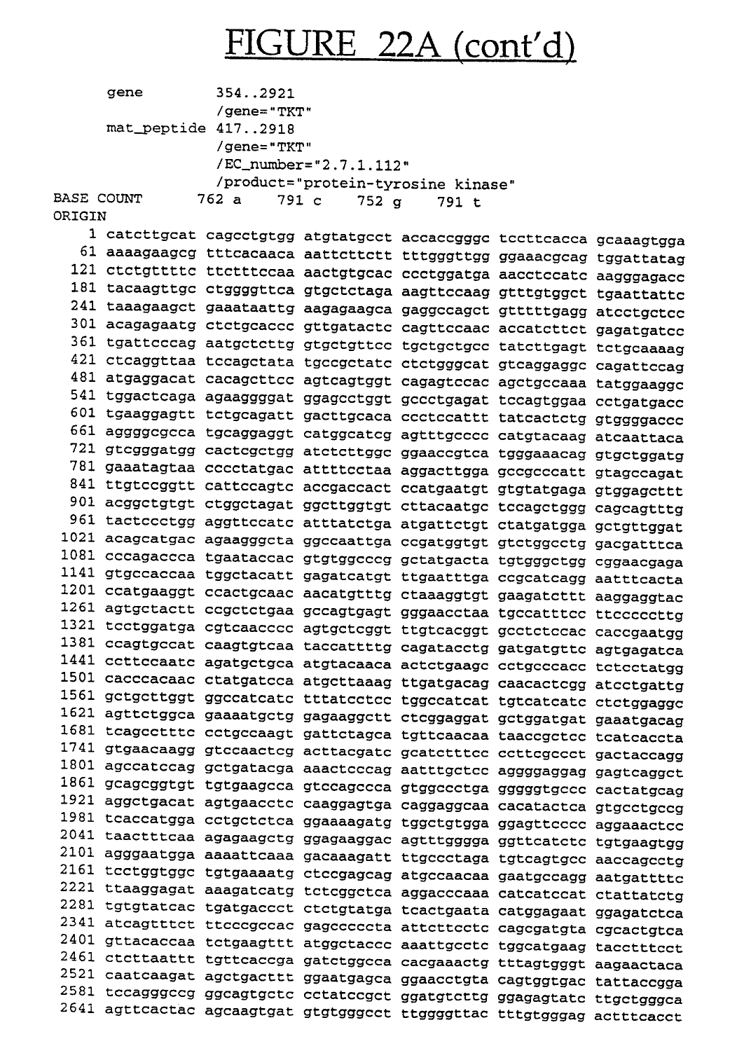

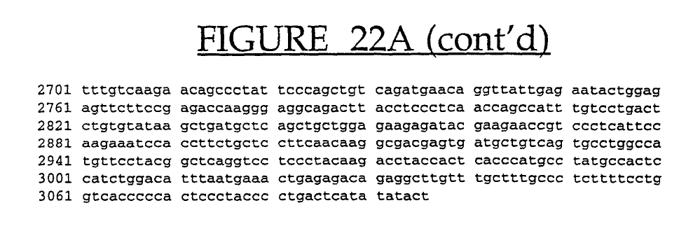

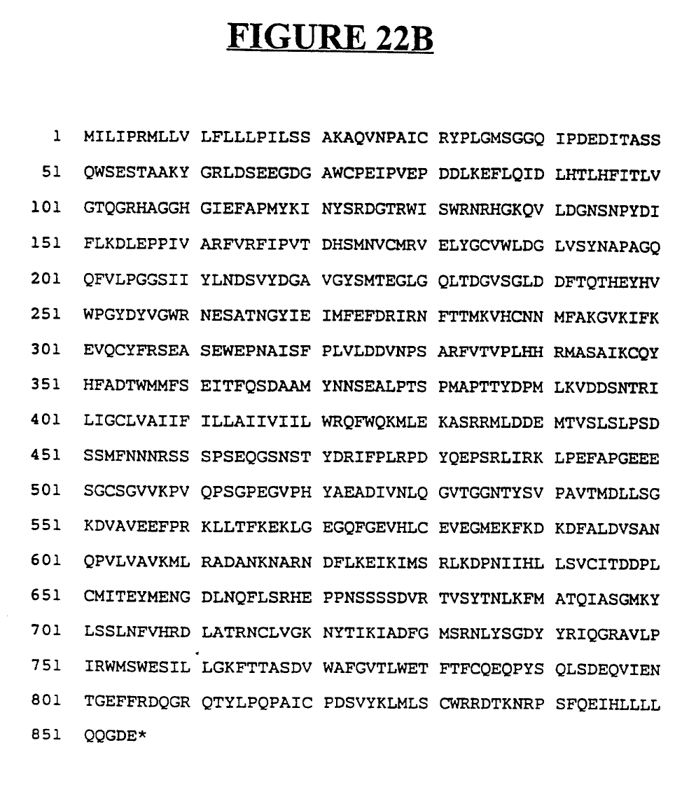

FIGS. 22A and 22B shows the nucleotide sequence and deduced amino acid sequence of a human DDR2 (i.e. TKT). (Genbank Accession No. X74764). The juxtamembrane region is between amino acid 422 and amino acid 570.

It will be appreciated that the receptor tyrosine kinase protein for use in the present invention, may be an isoform or a part of the protein. The isoforms contemplated for use in the methods of the invention are isoforms having the same functional properties as the discoidin domain receptor tyrosine kinase proteins.

In a preferred embodiment, the part of the protein has at least 20 contiguous amino acids and preferably comprises an extracellular domain or the C-terminal region. The receptors may also be oligomerized, in particular dimers and trimers are contemplated for use in the methods and compositions of the invention.

A part of a discoidin domain receptor tyrosine kinase protein includes a portion of the molecule that interacts directly or indirectly with a collagen or an intracellular molecule such as Shc, or a protein with a PDZ domain (i.e. a binding domain). A binding domain may be a sequential portion of the molecule i.e. a contiguous sequence of amino acids, or it may be conformational i.e. a combination of non-contiguous sequences of amino acids which when the molecule is in its native state forms a structure that interacts with another molecule in a complex of the invention. A part of a DDR protein contemplated herein includes a molecular entity which is identical or substantially equivalent to the native binding domain of a molecule in a complex of the invention (i.e. DDR, or part thereof and a collagen and a part thereof). Peptides derived from binding domains are discussed below.

A DDR protein used in the invention may be a protein having substantial sequence identity with the sequence of a discoidin domain receptor tyrosine kinase protein. The term “sequence having substantial identity” means those amino acid sequences having slight or inconsequential sequence variations from the sequence of discoidin domain receptor tyrosine kinase protein. The variations may be attributable to local mutations or structural modifications. Suitable proteins may have over 75%, preferably over 85%, most preferably over 90% identity with a discoidin domain receptor tyrosine kinase protein.

A discoidin domain receptor tyrosine kinase or part thereof, may be selected for use in the present invention based on the nature of the ligand which is targeted or selected. The selection of a particular ligand and complementary discoidin domain receptor tyrosine kinase provides specific complexes and in the methods of the invention allows for the identification of specific substances that affect a discoidin domain receptor tyrosine kinase regulatory pathway. For example, a type I, II, III, IV or V collagen may be interacted with DDR1 in the complexes and methods of the invention. A type I or III collagen may also be interacted with DDR2 in the complexes and methods of the invention.

A discoidin domain receptor tyrosine kinase or part thereof may be isolated from cells, which are known to express the proteins (e.g.DDR1 may be isolated from neuroepithelial cells during mouse embryonic development, or human ovarian and breast cancer samples). Alternatively the protein or part of the protein may be prepared using recombinant DNA methods known in the art. By way of example, nucleic acid molecules having a sequence which codes for a discoidin domain receptor tyrosine kinase protein, or a part of the protein may be prepared and incorporated in a known manner into an appropriate expression vector which ensures good expression of the protein or part thereof. Possible expression vectors include but are not limited to cosmids, plasmids, or modified viruses, so long as the vector is compatible with the host cell used.

The discoidin domain receptor tyrosine kinase protein or parts thereof may also be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

A discoidin domain receptor tyrosine kinase protein or parts thereof, for use in the methods of the present invention may be associated with a cell surface. Expression of a discoidin receptor tyrosine kinase protein or parts thereof, on cell surfaces can be carried out using conventional methods.

Conjugates of the protein, or parts thereof, with other molecules, such as proteins or polypeptides, may be prepared and used in the methods described herein. This may be accomplished, for example, by the synthesis of N-terminal or C-terminal fusion proteins. Thus, fusion proteins may be prepared by fusing, through recombinant techniques, the N-terminal or C-terminal of a discoidin domain receptor tyrosine kinase protein or parts thereof, and the sequence of a selected protein or marker protein with a desired biological function. Examples of proteins which may be used to prepare fusion proteins include immunoglobulins and parts thereof such as the constant region of an immunoglobulin, and lymphokines such as gamma interferon, tumor necrosis factor, IL-1, IL-2,IL-3, Il-4, IL-5, IL-6, IL-7, IL-8, IL-9, IL-10, IL-11, GM-CSF, CSF-1 and G-CSF.

The discoidin domain receptor tyrosine kinase protein, isoforms or parts thereof, employed in the invention may be insolubilized. For example, the receptor protein or part thereof, preferably the extracellular domain, may be bound to a suitable carrier. Examples of suitable carriers are agarose, cellulose, dextran, Sephadex, Sepharose, liposomes, carboxymethyl cellulose polystyrene, filter paper, ion-exchange resin, plastic film, plastic tube, glass beads, polyamine-methyl vinylether-maleic acid copolymer, amino acid copolymer, ethylene-maleic acid copolymer, nylon, silk, etc. The carrier may be in the shape of, for example, a tube, test plate, beads, disc, sphere etc. The insolubilized receptor tyrosine kinase protein may be prepared by reacting the material with a suitable insoluble carrier using known chemical or physical methods, for example, cyanogen bromide coupling.

Suitable collagens which may be used in the methods and compositions of the invention include type I, II, III, IV, and V collagen. Collagen may be obtained from a commercial source, or be produced using conventional methods. A collagen is selected for the complexes and methods described herein that provides for activation of a selected DDR. A part of the collagen may be used in the methods and compositions of the invention. In an embodiment of the invention, a carbohydrate moiety of a collagen or a portion of this moiety, or a G-X-Y repeat region having a triple helical conformation of a collagen, is used which is capable of binding to the extracellular domain of a discoidin domain receptor tyrosine kinase (preferably DDR2) and activating the receptor. A collagen or part thereof used in the invention may be insolubilized; for example, it may be bound to a suitable carrier as described herein.

Peptides

The invention provides peptide molecules which bind to and inhibit the interactions of a DDR or part thereof and a collagen or part thereof, or a DDR and an intracellular molecule such as Shc, or a protein having a PDZ domain. A peptide derived from a specific binding domain may encompass the amino acid sequence of a naturally occurring binding site, any portion of that binding site, or other molecular entity that functions to bind an associated molecule. A peptide derived from such a binding domain will interact directly or indirectly with an associated molecule in such a way as to mimic the native binding domain. Such peptides may include competitive inhibitors, enhancers, peptide mimetics, and the like. All of these peptides as well as molecules substantially homologous, complementary or otherwise functionally or structurally equivalent to these peptides may be used for purposes of the present invention.

“Peptide mimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of a peptide, or enhancer or inhibitor of the invention. Peptide mimetics also include peptoids, oligopeptoids (Simon et al (1972) Proc. Natl. Acad, Sci USA 89:9367); and peptide libraries containing peptides of a designed length representing all possible sequences of amino acids corresponding to a peptide of the invention.

Peptides may be synthesized by conventional techniques. For example, the peptides may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, supra, Vol 1, for classical solution synthesis.)

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors. (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

Peptides of the invention may be used to identify lead compounds for drug development. The structure of the peptides described herein can be readily determined by a number of methods such as NMR and X-ray crystallography. A comparison of the structures of peptides similar in sequence, but differing in the biological activities they elicit in target molecules can provide information about the structure-activity relationship of the target. Information obtained from the examination of structure-activity relationships can be used to design either modified peptides, or other small molecules or lead compounds which can be tested for predicted properties as related to the target molecule. The activity of the lead compounds can be evaluated using assays similar to those described herein.

Information about structure-activity relationships may also be obtained from co-crystallization studies. In these studies, a peptide with a desired activity is crystallized in association with a target molecule, and the X-ray structure of the complex is determined. The structure can then be compared to the structure of the target molecule in its native state, and information from such a comparison may be used to design compounds expected to possess desired activities.

Particular peptides which may be used in the invention include peptides derived from the sites on a DDR (e.g. DDR1b) that bind to Shc, or derived from the Shc PTB binding domain, peptides derived from the sites on a DDR that bind to insulin receptor substrate (IRS-1) or the sites on IRS-1 that bind to a DDR, or the sites on a DDR (e.g. DDR1) that bind to proteins with a PDZ domain, or a PDZ domain. In an embodiment, peptides comprising the amino acids ψXNPXpY are contemplated wherein ψ is a hydrophobic amino acid including alanine, phenylalanine, isoleucine, leucine, methionine, proline, valine, and tryptophan, X is any amino acid, N is Asn, P is proline, and pY is phosphotyrosine. Examples of specific peptides of the invention are LLSNPApY, ALLLSNPApYRLLA, and AEDALNTV (amino acids 906 to 913 of DDR1).

Complexes

The complexes of the invention include the following: (a) an isolated complex comprising a DDR or an isoform or part thereof, and a collagen or a part thereof; (b) an isolated complex comprising a DDR (e.g. DDR1b) and Shc or a PTB domain of Shc, and (c) an isolated complex comprising a DDR (e.g. DDR1) and a protien containing a PDZ domain or a PDZ domain. The DDR in a complex may be oligomerized, it may be conjugated to another protein, and/or it may be insolubilized. In addition, a collagen in a complex of the invention may be insolubilized. The complexes may comprise only the binding domains of the interacting molecules and such other flanking sequences as are necessary to maintain the activity of the complexes. Examples of complexes include DDR1 with types I, II, III, IV and V collagen, DDR2 with types I and III collagen, DDR1b and Shc, and DDR1 and a protein containing a PDZ domain.

The invention also contemplates antibodies specific for the complexes or peptides of the invention. The antibodies may be intact monoclonal or polyclonal antibodies, and immunologically active fragments (e.g. a Fab or (Fab)₂ fragment), an antibody heavy chain, and antibody light chain, a genetically engineered single chain Fᵥ molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies specific for the complexes of the invention may be used to detect the complexes in tissues and to determine their tissue distribution. In vitro and in situ detection methods using the antibodies of the invention may be used to assist in the prognostic and/or diagnostic evaluation of conditions such as proliferative disorders. Antibodies specific for the complexes of the invention may also be used therapeutically as discussed herein.

Some genetic diseases may include mutations at the binding domain regions of the interacting molecules in the complexes of the invention. Therefore, if a complex of the invention is implicated in a genetic disorder, it may be possible to use PCR to amplify DNA from the binding domains to quickly check if a mutation is contained within one of the domains. Primers can be made corresponding to the flanking regions of the domains and standard sequencing methods can be employed to determine whether a mutation is present. This method does not require prior chromosome mapping of the affected gene and can save time by obviating sequencing the entire gene encoding a defective protein.

Evaluating and Identifying Substances

The methods described herein may be used to identify substances that modulate a DDR tyrosine kinase-mediated signaling pathway, and in particular modulating extracellular matrix synthesis, degradation, or remodelling. Novel substances are contemplated that bind to molecules in the complexes of the invention, or bind to other molecules that interact with the molecules. Substances that interfere with or enhance the interaction of the molecules in a complex of the invention, or other proteins that interact with the molecules are also contemplated.

The substances identified using the methods of the invention include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal humanized, anti-idiotypic, chimeric, single chain antibodies, fragments, (e.g. Fab, F(ab)₂, and Fab expression library fragments, and epitope-binding fragments thereof)], and small organic or inorganic molecules. The substance may be an endogenous physiological compound or it may be a natural or synthetic compound.

The invention contemplates a method for evaluating a test substance for its ability to affect a DDR tyrosine kinase-mediated signaling pathway, and in particular to modulate extracellular matrix synthesis, degradation, or remodelling by assaying for an agonist or antagonist (i.e. enhancer or inhibitor) of the binding of the molecules in a complex of the invention. The method generally involves preparing a reaction mixture containing the molecules in the complex and the test substance under conditions which permit the formation of complexes. The test substance may be initially added to the mixture, or may be added subsequent to the addition of the molecules. Control reaction mixtures without the test substance or with a placebo are also prepared. The formation of complexes is detected, and the formation of complexes in the control reaction but not in the reaction mixture indicates that the test substance interferes with the interaction of the molecules. The reactions may be carried out in the liquid phase, or the molecules or the test substance may be immobilized as described herein. Substances identified using the methods of the invention may be isolated, cloned and sequenced using conventional techniques.

In an embodiment, a method is provided for identifying a substance which affects a DDR tyrosine kinase-mediated signaling pathway, comprising the steps of:

(a) reacting a collagen, and at least one discoidin domain receptor tyrosine kinase protein, or an isoform or a part of the protein, and a test substance, wherein the collagen and discoidin domain receptor tyrosine kinase protein are selected so that they bind to form a collagen-discoidin domain receptor tyrosine kinase protein complex; and

(b) comparing to a control in the absence of the substance to determine the effect of the substance.

In particular, a method is provided for identifying a substance which affects a DDR tyrosine kinase-mediated signaling pathway in a cell, comprising

(a) reacting a collagen or part thereof, and at least one discoidin domain receptor tyrosine kinase protein, or an isoform or a part of the protein, and a test substance, wherein the collagen and discoidin domain receptor tyrosine kinase protein are selected so that they bind to form a collagen-discoidin domain receptor tyrosine kinase protein complex, under conditions which permit the formation of collagen-discoidin domain receptor tyrosine kinase protein complexes, and

(b) assaying for complexes, for free substance, for non-complexed collagen, or for activation of the protein.

Conditions which permit the formation of complexes may be selected having regard to factors such as the nature and amounts of the substance and the ligand.

The complexes, free substance or non-complexed ligand may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against the substance, or a labelled collagen, or a labelled substance may be utilized. Antibodies, receptor protein or substance may be labelled with a detectable substance as described above.

Activation of the protein may be assayed by measuring tyrosine phosphorylation of the protein, oligomerization of the protein, binding of a PTB domain to the discoidin domain receptor tyrosine kinase protein juxtamembrane domain, or by assaying for a biological affect on the cell, such as inhibition or stimulation of proliferation, differentiation, or migration.

It will be understood that the agonists and antagonists i.e. inhibitors and enhancers that can be assayed using the methods of the invention may act on one or more of the binding sites on the interacting molecules in the complex including agonist binding sites, competitive antagonist binding sites, non-competitive antagonist binding sites or allosteric sites.

The invention also makes it possible to screen for antagonists that inhibit the effects of an agonist of the interaction of molecules in a complex of the invention. Thus, the invention may be used to assay for a compound that competes for the same binding site of a molecule in a complex of the invention.

The invention also contemplates methods for identifying novel compounds that bind to proteins that interact with a molecule of a complex of the invention thereby affecting a DDR-signaling pathway. Protein-protein interactions may be identified using conventional methods such as co-immunoprecipitation, crosslinking and co-purification through gradients or chromatographic columns. Methods may also be employed that result in the simultaneous identification of genes which encode proteins interacting with a molecule. These methods include probing expression libraries with labeled molecules. Additionally, x-ray crystallographic studies maybe used as a means of evaluating interactions with substances and molecules. For example, purified recombinant molecules in a complex of the invention when crystallized in a suitable form are amenable to detection of intra-molecular interactions by x-ray crystallography. Spectroscopy may also be used to detect interactions and in particular, Q-TOF instrumentation may be used.

Two-hybrid systems may also be used to detect protein interactions in vivo. Generally, plasmids are constructed that encode two hybrid proteins. A first hybrid protein consists of the DNA-binding domain of a transcription activator protein fused to a molecule in a complex of the invention, and-the second hybrid protein consists of the transcription activator protein's activator domain fused to an unknown protein encoded by a cDNA which has been recombined into the plasmid as part of a cDNA library. The plasmids are transformed into a strain of yeast (e.g. S. cerevisiae) that contains a reporter gene (e.g. lacZ, luciferase, alkaline phosphatase, and horseradish peroxidase) whose regulatory region contains the transcription activator's binding site. The hybrid proteins alone cannot activate the transcription of the reporter gene. However, interaction of the two hybrid proteins reconstitutes the functional activator protein and results in expression of the reporter gene, which is detected by an assay for the reporter gene product.

It will be appreciated that fusion proteins and recombinant proteins may be used in the above-described methods. It will also be appreciated that the complexes of the invention may be reconstituted in vitro using recombinant molecules and the effect of a test substance may be evaluated in the reconstituted system.

The reagents suitable for applying the methods of the invention to evaluate substances and compounds that affect or modulate a DDR receptor tyrosine kinase-mediated signaling pathway may be packaged into convenient kits providing the necessary materials packaged into suitable containers. The kits may also include suitable supports useful in performing the methods of the invention.

Compositions and Treatments

The above mentioned methods of the invention may be used to identify substances that affect a discoidin domain receptor tyrosine kinase signaling pathway in a cell, particularly those involved in proliferation, metastasis, or extracellular matrix synthesis, degradation, or remodelling. It will be appreciated that such substances will be useful as pharmaceuticals to modulate proliferation, metastasis, and/or extracellular matrix synthesis, degradation, or remodelling. The ability of substances identified using the methods of the invention to affect proliferation and/or metastasis and other cellular processes may be confirmed in animal models. For example, the MDAY-D2 murine model may be used to confirm the utility of a substance as an anti-proliferative or anti-metastatic agent.

The invention provides a method for treating or preventing a condition involving a discoidin domain receptor tyrosine kinase-mediated signaling pathway, which method comprises administering to a patient in need thereof an amount of a substance which is effective to interfere with (i.e. inhibit or enhance) the signaling pathway wherein the substance is (a) a discoidin domain receptor tyrosine kinase or part thereof; (b) a collagen or part thereof; (b) an isolated complex comprising a DDR or a part thereof, and a collagen or a part thereof; (c) peptides derived from the binding domain of a DDR that interacts with a collagen or part of a collagen, or with Shc or a protein containing a PDZ domain; (d) a molecule derived from the binding domain of collagen that interacts with a DDR or a part thereof, (e) antibodies specific for the complexes and peptides; or (f) a substance first identified by

(i) reacting a collagen, and at least one discoidin domain receptor tyrosine kinase protein, or an isoform or a part of the protein, and the test substance, wherein the collagen and discoidin domain receptor tyrosine kinase protein are selected so that they bind to form a collagen-discoidin domain receptor tyrosine kinase protein complex; and

(ii) comparing to a control in the absence of the substance to determine the effect of the substance.

The invention also relates to a pharmaceutical composition which comprises (a) a discoidin domain receptor tyrosine kinase or part thereof; (b) a collagen or part thereof, preferably a carbohydrate moiety; (b) an isolated complex comprising a DDR or a part thereof, and a collagen or a part thereof; (c) peptides derived from the binding domain of a DDR that interacts with a collagen or part of a collagen, or with Shc or a protein containing a PDZ domain ; (d) a molecule derived from the binding domain of collagen that interacts with a DDR or a part thereof; (e) antibodies specific for the complexes, peptides, and molecules of (b), (c) or (d); or, (f) a substance-first identified by a method of the invention, in an amount effective to affect a discoidin domain receptor tyrosine kinase-mediated signaling pathway, and a pharmaceutically acceptable carrier, diluent or excipient. In an embodiment of the invention, the composition may comprise an extracellular domain of an discoidin domain receptor tyrosine kinase, or the portion of the extracellular domain which binds to a G-X-Y repeat region or a carbohydrate moiety of a collagen, or oligomers or mimetics thereof.

The method and compositions of the invention may be used to alter proliferation or metastasis in a mammal, treat conditions involving structural or functional deregulation of collagens such as Sickler syndrome, treat conditions involving defects in collagen such as osteogenesis imperfecta, treat conditions requiring modulation of extracellular matrix degradation or remodelling, enhance wound healing, and enhance cartilage or bone formation. The compositions of the invention are administered to subjects in a biologically compatible form suitable for pharmaceutical administration in vivo. By “biologically compatible form suitable for administration in vivo” is meant a form of the protein to be administered in which any toxic effects are outweighed by the therapeutic effects of the protein. The term subject is intended to include mammals. Examples of subjects include humans, dogs, cats, mice, rats, and transgenic species thereof. Administration of a therapeutically active amount of the pharmaceutical compositions of the present invention is defined as an amount effective, at dosages and for periods of time necessary to achieve the desired result. For example, a therapeutically active amount of an active substance may vary according to factors such as the condition, age, sex, and weight of the individual. Dosage regimes may be adjusted to provide the optimum therapeutic response. For example, several divided doses may be administered daily or the dose may be proportionally reduced as indicated by the exigencies of the therapeutic situation.

The active compound (e.g., protein) may be administered in a convenient manner such as by injection (subcutaneous, intravenous, etc.), oral administration inhalation, transdermal application or rectal administration. Depending on the route of administration, the active compound may be coated in a material to protect the compound from the action of enzymes, acids and other natural conditions which may inactive the compound. The pharmaceutical compositions of the invention can be for oral, local, inhalant or intracerebral administration. Preferably, the pharmaceutical compositions of the invention are administered directly to the peripheral or central nervous system, for example by administration intracerebrally.

The pharmaceutical composition of the invention can be administered to a subject in an appropriate carrier or diluent, co-administered with enzyme inhibitors or in an appropriate carrier such as microporous or solid beads or liposomes. The term “pharmaceutically acceptable carrier” as used herein is intended to include diluents such as saline and aqueous buffer solutions. Liposomes include water-in-oil-in-water emulsions as well as conventional liposomes (Strejan et al., (1984) J. Neuroimmunol 7:27). The active compound may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, asorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (e.g., antibody) plus any additional desired ingredient from a previously sterile-filtered solution thereof.

When the active compound is suitably protected, as described above, the composition may be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

It is also contemplated that the pharmaceutical compositions of the invention may comprise cells or viruses, preferably retroviral vectors, transformed with nucleic acid molecules encoding a purified and isolated discordin domain receptor tyrosine kinase protein, or an isoform or a part of the protein, a peptide of the invention, an antibody to a complex of the invention, or a substance identified using the methods of the invention, such that they express the protein, isoform, or a part of the protein, preferably the extracellular domain, or substance in vivo. Viral vectors suitable for use in the present invention are well known in the art including recombinant vaccinia viral vectors (U.S. Pat. Nos. 4,603,112 and 4,769,330), recombinant pox virus vectors (PCT Publication No. WO 89/01973), and preferably, retroviral vectors (“Recombinant Retroviruses with Arnphotropic and Ecotropic Host Ranges,” PCT Publication No. WO 90/02806; “Retroviral Packaging Cell Lines and Processes of Using Same,” PCT Publication No. WO 89/07150; and “Antisense RNA for Treatment of Retroviral Disease States,” PCT Publication No. WO 87/03451). The compositions containing cells or viruses may be directly introduced into a subject. Nucleic acid molecules encoding a DDR or an isoform or part of the protein, a peptide of the invention, an antibody to a complex of the invention, or a substance identified using the methods of the invention, may also be introduced into a subject using physical techniques such as microinjection and electroporation or chemical methods such as coprecipitation and incorporation of nucleic acids into liposomes. They may also be delivered in the form of an aerosol or by lavage.

The following non-limiting examples are illustrative of the present invention:

### EXAMPLES

### Example 1

The present inventors have shown that the activated b-isoform of DDR1 (MCK10), but not the a-isoform, associates with Shc in vivo and binds to the Shc PTB domain in vitro. This interaction is blocked by a phosphopeptide containing the LLSNPAY motif, which is specifically found in the juxtamembrane insert of the b-isoform. These results suggest that alternative splicing directly regulates the ability of DDR1 to interact with Shc by controlling the presence or absence of a PTB-binding site.

Experimental Procedures

Materials and cell lines—The cloning and expression of the functional Shc domains as glutathione S-transferase (GST) fusion proteins have been described (13). Briefly, the PTB domain fusion construct comprises amino acids 1-225 of human p52 Shc and the SH2 domain construct spans amino acids 366-473. The DDR1 (also known as MCK10) expression vectors have been described previously (1). The phosphopeptide ALLLSNPApYRLLLA was synthesized using an Applied Biosystems model 431A instrument. Antibodies to Shc were raised against a GST-Shc SH2 fusion protein. Other antibodies were purchased from Santa Cruz, Inc. (monoclonal antiphosphotyrosine antibody 4G10, and polyclonal rabbit serum against amino acid 894-913 of MCK10). Human embryonic kidney fibroblast 293 cells were obtained from the American Tissue Culture Collection (ATCC CRL 1573) and cultivated under the recommended conditions.

Transient expression—Semiconfluent 293 cells were transfected by calcium-phosphate precipitation with a cytomegalovirus-based expression vector containing the a- or b-isoform of DDR1 (1). Sixteen hours later, cells were transferred to serum-free medium for a further 24 h. Prior to lysis, cells were stimulated with 1 mM orthovanadate (pH 10.0) for 90 min. For in vivo labeling of phosphorylated proteins, cells were grown with 0.5 mCi ml-1 [32P]-inorganic phosphate (NEN) for 4 h.

Immunoprecipitation, Western blotting and kinase assay—Transfected 293 cells were lysed in NP40 buffer containing 20 mM Tris-HCl (pH 8.0), 150 mM NaCl, 2 mM EDTA, 1% NP40, 10 mM NaF, 1 mM phenylmethylsulfonyl fluoride, 1 mM orthovanadate and 10 μgml⁻¹ aprotinin. The cellular lysates were centrifuged 10 min at 4° C. and 13000 rpm and aliquots of the supernatant were subjected to SDS-PAGE or further analysed by immunoprecipitation with specific antibodies for 3 h at 4° C. on a rotating wheel. The immunocomplex was washed three times with NP40-buffer and analysed by SDS-PAGE. Proteins were transferred to a nitrocellulose membrane (Schleicher & Schuell) and immunoblotted with antibodies diluted 1:500 in 50 mM Tris-HCl (pH 7.5), 150 mM NaCl, 5 mM EDTA, 0.05% Triton X-100, 0.25% gelatin overnight. Western blots were developed using horseradish peroxidase-coupled secondary goat anti-protein A antibody (Biorad) and enhanced chemiluminescence (Amersham). For reprobing, the membrane was stripped in 70 mM Tris-HCl (pH 6.8), 2% SDS, 0.1% β-mercaptoethanol at 55° C. for 30 min. For in vitro kinase assays, immunoprecipitates were washed twice in 40 mM HEPES (pH 7.5), 20 mM MgCl₂, 2 mM MnCl₂, 10 μM orthovanadate, 5 μM dATP and incubated with 20 μCi [γ-³²P]-ATP at 30° C. for 20 min. The products of the kinase reaction were monitored by SDS-PAGE, transferred to immobilon membrane (NEN) and autoradiography.

Phosphopeptide mapping—proteins were cut out from the membrane and digested with N-p-tosyl-L-lysine-chloromethyl-ketone treated trypsin (Sigma) for 16 h, oxidized with performic acid for 1 h, stepwise desalted and concentrated by lyophilisation. Equal cpm of radiolabeled peptides were separated in two dimensions, using electrophoresis on thin layer cellulose (TLC) plates (100 mm, Merck) with a HTLE 7000 apparatus (C.B.S. Inc., Del Mar, Calif., USA) in pH 1.9 buffer (2.5% formic acid, 7.8% acidic acid) in the first dimension, and ascending chromatography (37.5% n-butanol, 25% pyridine, 7.5% acidic acid, 30% water) in the second dimension. TLC-plates were dried and exposed to X-ray film at −70° C. using an intensifying screen.

Surface plasmon resonance—experiments were done with a BIAcore instrument (Pharmacia). A phosphopeptide corresponding to the NPXY sequence in the polyomavirus middle T antigen (mT) (LSLLSNPTpYSVNSK) was absorbed to the surface of a biosensor chip. Binding of soluble GST-Shc PTB domain fusion protein to the mT peptide was measured in the presence or absence of competing soluble phosphopeptides based on the sequences around Tyr 513 of DDR1b or Tyr 490 of the NGFR (HIIENPQpYFSD) (21).

Results And Discussion

To determine whether the DDR1 RTK interacts with Shc proteins, human embryonic kidney fibroblast 293 cells were transfected with expression vectors encoding the DDR1a or DDR1b isoforms. Receptor kinase activation and autophosphorylation were achieved by treating the cells with 1 mM orthovanadate 90 min prior to lysis. Orthovanadate treatment has been previously shown to stimulate DDR1 tyrosine phosphorylation in vivo (I). Shc proteins were immunoprecipitated from lysates of these cells, and the immunoprecipitates were immunoblotted with anti-phosphotyrosine antibody. A tyrosine phosphorylated protein of approximately 125 kDa coprecipitated with Shc from DDR1b transfected cells (FIG. 1A). Reprobing of the blot with anti-DDR1 antibody demonstrated that the 125 kDa Shc-associated protein is DDR1b (FIG. 1B). No association was seen between DDR1a and Shc in 293 cells, despite the fact that DDR1a itself became tyrosine phosphorylated to a comparable extent to DDR1b (FIG. 1D).

To investigate the interaction of Shc with DDR1b in more detail the Shc PTB and SH2 domains were expressed as GST-fusion proteins. These GST-fusions were immobilized and incubated with lysates of 293 cells, which have been transfected with the DDR1a or DDR1b isoforms and incubated with orthovanadate to induce receptor tyrosine phosphorylation. The b-isoform of DDR1 from orthovanadate-treated cells specifically associated with the Shc PTB domain in vitro, whereas no binding of the b-isoform from unstimulated cells was observed (FIG. 1E). In contrast, the Shc PTB domain failed to bind to DDR1a from orthovanadate-stimulated cells. No binding of the Shc SH2 domain to tyrosine phosphorylated DDR1a or DDR1b was detected. These results suggest that the Shc PTB domain recognizes an autophosphorylation site which is specific to DDR1b and absent from DDR1a, consistent with the presence of the LLSNPAY motif in the juxtamembrane insert of DDR1b. Thus, alternative splicing apparently regulates the presence or absence of a Shc PTB-binding site in DDR1, and thereby controls the ability of DDR1 to interact with this downstream target in vivo.

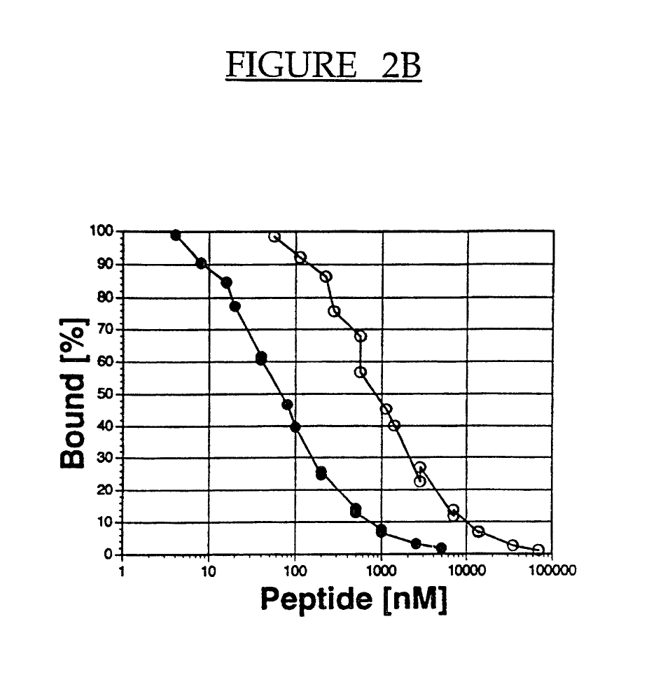

The results presented above raise the possibility that the Shc PTB domain binds to phosphorylated Tyr 513, which lies within the motif LLSNPAY in the DDR1b juxtamembrane insert. To test whether phosphorylation of Tyr 513 might form a PTB-binding site, a 14-mer phosphopeptide with the sequence ALLLSNPApYRLLLA was synthesized, corresponding to residues 505-518 of DDR1b. The ability of this phosphopeptide to bind the Shc PTB domain was analysed using a competition assay, in which the capacity of GST-Shc PTB fusion protein to bind tyrosine phosphorylated DDR1b from lysates of transfected 293 cells was measured in the presence of increasing amounts of the Tyr 513 phosphopeptide. 100 nM phosphopeptide inhibited binding of the Shc PTB domain to DDR1b, and full inhibition was achieved at a phosphopeptide concentration of 2 μM (FIG. 2A). These results suggest that phosphorylation of Tyr 513 creates a strong binding site for the Shc PTB domain. To quantitate this interaction more precisely, surface plasmon resonance technology was used, in which soluble DDR1b phosphopeptide was employed to inhibit binding of the GST-Shc PTB fusion protein to a polyomavirus middle T antigen phosphopeptide, immobilized on a biosensor chip (21). As shown in FIG. 2B, a concentration of approximately 800 nM DDR1b phosphopeptide, containing the sequence around Tyr 513, inhibited binding of the Shc PTB domain to the middle T antigen phosphopeptide by 50% (IC₅₀). These findings contrast with an IC₅₀ value of approximately 100 nM found when a phosphopeptide around the NPXY motif of the NGF receptor was used in a comparable experiment (FIG. 2B), but are similar to the IC₅₀ of the middle T antigen phosphopeptide itself (21).

Experimental data analysing the interaction between the Shc PTB domain and phosphorylated peptides have emphasized the importance of specific residues N-terminal to the tyrosine phosphorylation site.

Phosphopeptides which lack an Asn at the −3 position are ineffective in binding the Shc PTB domain, while the Pro at the −2 position appears significant, but not essential for binding (21-25). A substantial decrease in affinity was also observed upon switching the hydrophobic residue at position −5 to a polar amino acid, indicating that bulky hydrophobic residues upstream of the core NPXY sequence contribute to the recognition of the Shc PTB domain (13, 18). Structural analysis of the Shc PTB domain bound to a phosphopeptide from the NGF receptor has identified a hydrophobic pocket that accomodates the −5 residue, explaining the preference for a hydrophobic residue at this position (26). The sequence preceeding Tyr 513 of DDR1b displays a triplet of leucines in the −5, −6 and −7 positions as well as an alanine at the −8 position, consistent with the finding that this site binds the Shc PTB domain with high affinity. This sequence matches the consensus not only for Shc PTB binding but also for interaction with the PTB domain of the insulin-receptor-substrate 1 (IRS-1), which requires hydrophobic residues at the −6 to −8 positions (27, 28). IRS-1 may associate with DDR1b. Interestingly, three leucines are also found at the +2 to +4 positions C-terminal to the NPXY motif in DDR1b. The significance of this highly symmetrical sequence is not clear. Perhaps the formation of an extended loop, initiated by the b-turn at the proline, is favored by these two blocks of triple leucines.

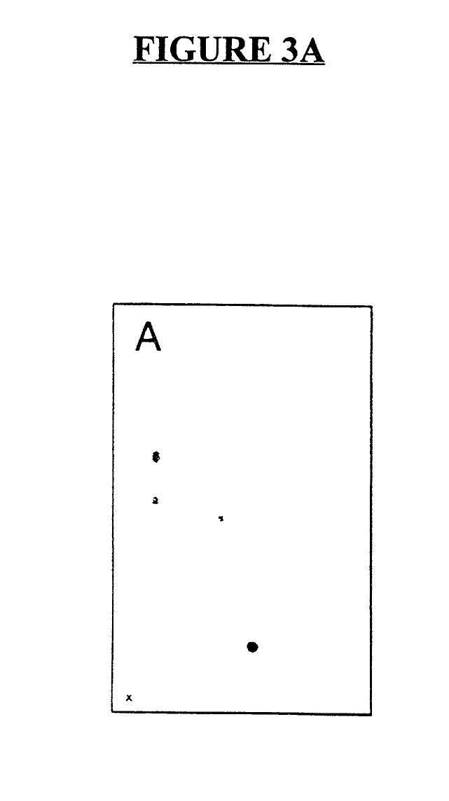

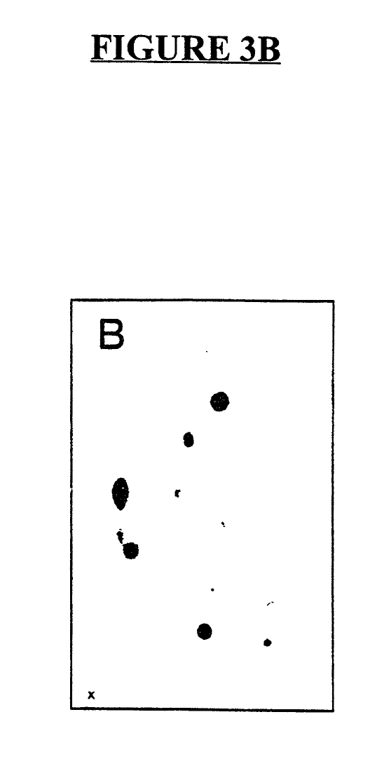

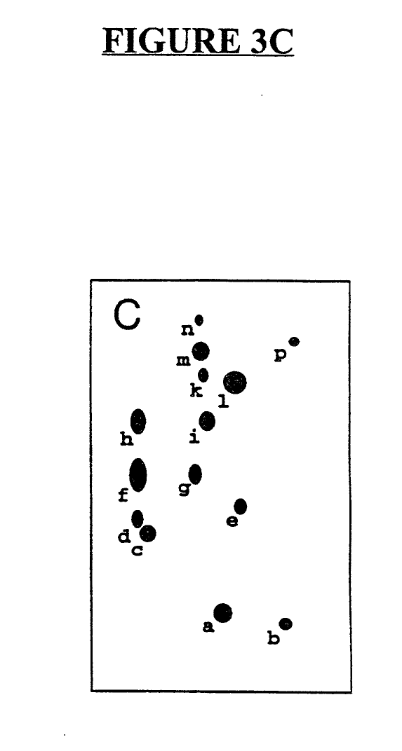

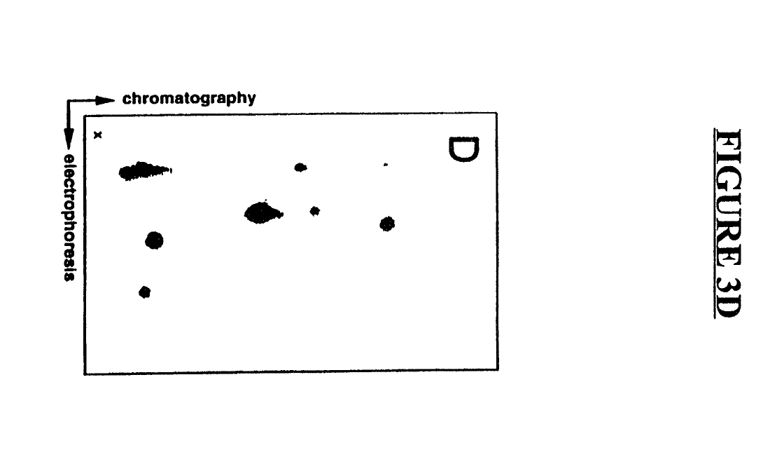

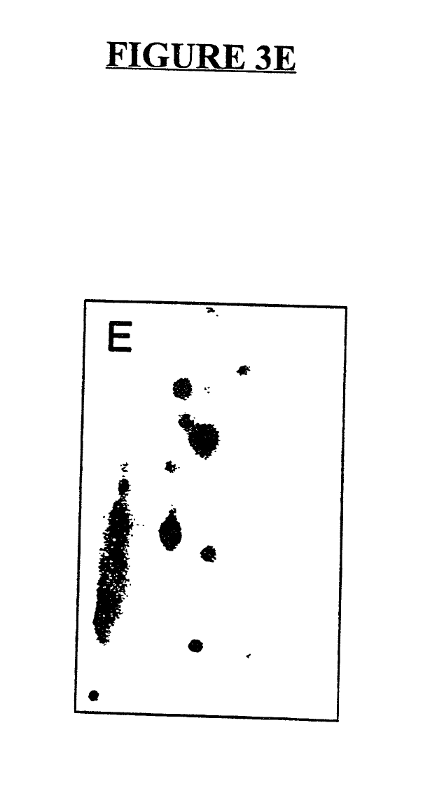

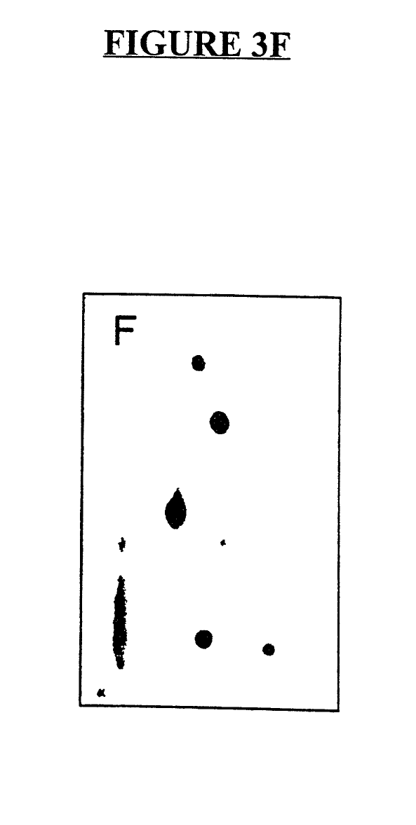

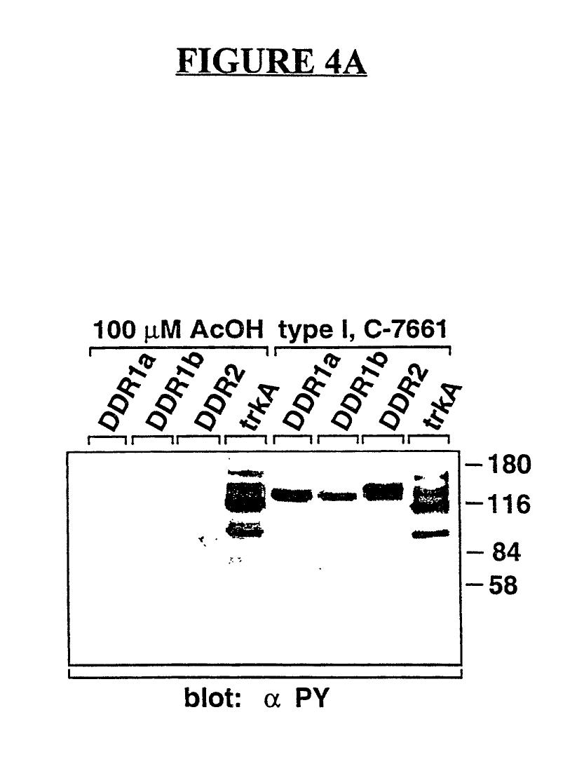

The sequence of the juxtamembrane insert unique to the DDR1b isoform contains two potential tyrosine phosphorylation sites, Tyr 513 and 520. The experiments detailed above show that phosphorylation of Tyr 513 can form a strong binding site for the Shc PTB domain. To monitor the extent of phosphorylation of the different DDR1 isoforms, 293 cells were transfected with expression vectors encoding the a- or b-isoforms of DDR1, and the transfected cells were metabolically labeled with [³²P]-orthophosphate. After stimulation with orthovanadate for 90 min, DDR1a or DDR1b isoforms were immunoprecipitated from cell lysates, purified by SDS-PAGE and subjected to tryptic digestion. The resulting tryptic phosphopeptides were separated in two dimensions. The two-dimensional phosphopeptide map of either receptor isoform showed rather complex patterns of spots, indicating phosphorylation at multiple sites. (FIGS. 3A and B). Superimposition of both maps indicated that all the phosphopeptides derived from DDR1a comigrated with peptides from DDR1b, whereas the spots c, e, i and I present in the tryptic phosphopeptide map of DDR1b were absent from the digest of DDR1a (FIGS. 3A, B and C). Among these DDR1b-specific phosphopeptides, spot 1 was the most intense, suggesting that it is derived from a major phosphorylation site in the cytoplasmic region of the receptor. This result is consistent with the possibility that the alternatively spliced insert of DDR1b provides novel autophosphorylation sites, which are not found in DDR1a. The presence of multiple phosphopeptides specific to the b-isoform could be explained by the phosphorylation of multiple sites within the juxtamembrane insert, or by the production of several distinct peptides containing a single phosphorylation site.

To pursue these results anti-DDR1 immunoprecipitations were subjected to in vitro kinase reactions to induce receptor autophosphorylation. Tryptic phosphopeptide maps of in vitro phosphorylated DDR1a and DDR1b (FIGS. 3D and E) or a mix of both isoforms (FIG. 3F) demonstrated that all of the phosphopeptides from the a-isoform were also present in digests of the b-isoform, but also showed that the longer isoform contained additional phosphopeptides. Two of the phosphopeptides specific to the b-isoform, the major spot 1 and more minor spot e, corresponded to phosphopeptides unique to the b-isoform isolated from ³²P-labeled orthovanadate-treated cells (FIGS. 3B and E). The in vitro kinase reaction also resulted in the phosphorylation of one DDR1b specific peptide (spot p), which was not seen in digests of in vivo-labeled protein, while phosphorylation of peptide c and i only occurred under in vivo conditions. These data suggest that DDR1b contains at least one novel autophosphorylation site in comparison with DDR1a, which is phosphorylated both in an in vitro autokinase reaction and in DDR1b-expressing cells following orthovanadate treatment. These results are compatible with the suggestion that the juxtamembrane insert of DDR1b modifies the interaction of DDR1 with its targets, specifically by providing a docking site for the Shc PTB domain.

### Example 2

Thus far, no ligand binding to DDR1 or DDR2 has been reported, nor any peptide or protein, which would trigger the intrinsic tyrosine kinase activity of the DDRs. Certain members of the collagen family have been identified as ligands for DDR1 and DDR2. Collagen was found to directly interact with the extracellular domains and collagen evoked tyrosine phosphorylation of DDRs in a time and concentration dependent manner.

Originally, a commercially available preparation of extracellular matrix proteins, called Matrigel, was found to induce tyrosine phosphorylation of DDR1. Testing various components of Matrigel, collagen type IV was identified to have ligand activity. Subsequently, nearly all commercially available collagen types from various organs and species (human, rat, mouse, bovine) were tested in the ligand assay. Collagen type I, III, IV and V were equally good ligands for DDR1. Collagen type I and III are highly potent ligands for DDR2, collagen types II and V show moderate, type IV no activity. When adding collagen type I into the tissue culture media of human embryonic kidney fibroblast 293 cells transfected with DDR1 or DDR2 cDNA, ligand activity was detected in a minimal concentration of app. 250 ng collagen per ml media. Maximal tyrosine phosphorylation was seen 90 min after stimulation with 10 μg/ml collagen type I. Previously T-47D, a human mammary carcinoma cell line, and A431, a human epidermoid carcinoma cell line, were found to display endogenous expression of DDR1. After stimulation with collagen type I, DDR1 protein was extracted by immunoprecipitation. In these immunoprecipitates, a significant increase in DDR1 tyrosine phosphorylation was detected after collagen stimulation using Western blot or in vitro kinase assay techniques.

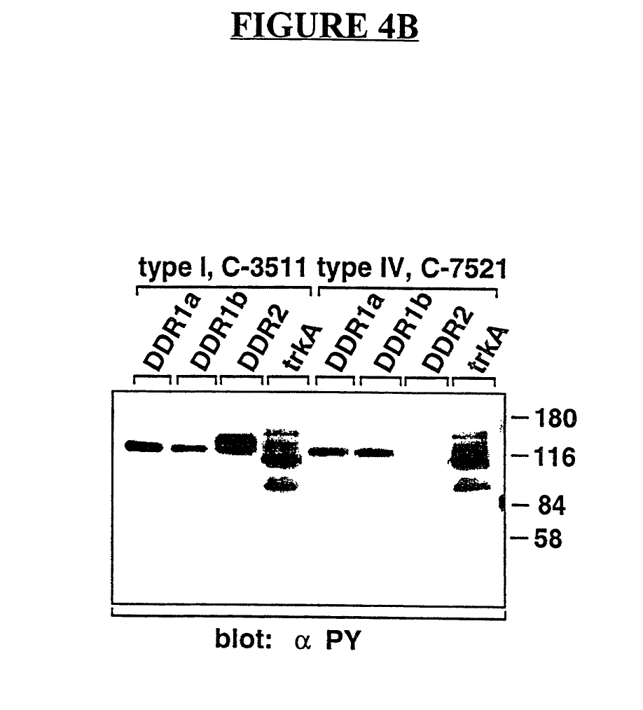

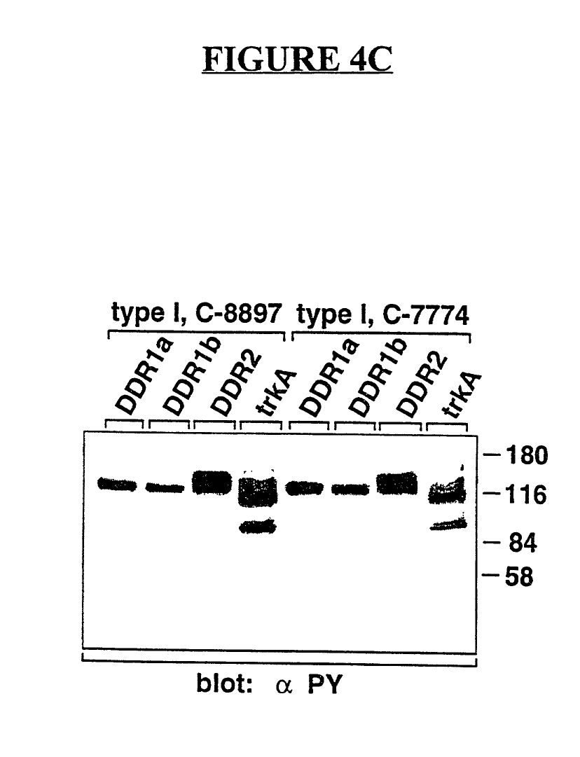

The ligand activity was abolished after pretreatment of collagen type I with collagenase, but not after treatment with pepsin. After removal of parts of the carbohydrate moiety of collagen type I using periodate as oxidants, the ligand activity for DDR2 was dramatically reduced. These data suggest that glycosylation of collagen is essential for DDRs activation. FIGS. 4, 5 and 6, and 12C illustrate the experimental results.

Collagens are extensively postranslational modified, e.g. hydroxylated, glycosylated and disulphid-linked. These modifications may be important or essential for the ligand activity.

In the progress of carcinogenesis and metastasis, the initial steps in tumor growth are still rather mysterious. Especially in metastasis, it is unclear, what induces the detachment of cells from a primary tumor, what factors are necessary for these cells to break the barrier of basement membranes and connective tissues surrounding the tumor and what factors are involved in reattachment at the site of metastasis. To initiate tumor invasion and metastatic growth, healthy basement membranes have to be brocken down. The family of matrix metallopropteinases (MMP) is known to be involved in this process. DDRs activation may correlate with the function of MMP. Because DDR1 is expressed on the surface of tumor cells, DDR2 is expressed in surronding stromal tissue and both are cabable of binding to collagens, these receptors may be involved in tumor growth and metastasis.

A pleiotropa of different human diseases are known to be linked to structural modification or functional deregulation of collagens. The genetic basis of many hereditary connective tissue disorders is unresolved. Mutations in the ligand binding domain, in the kinase domain or in any other position of the DDR1 and DDR2 genes may cause these kinds of disorders. DNA samples from patients with connective tissue disorders can be analyzed using Southern blot and PCR technology to identify genetic mutations in DDR1 and DDR2 genes. The human locus for DDR1 is 6p21.3, for DDR2 1q21-q23. A database search indicated several diseases, which are mapping close to these loci and are showing bone, skin or cartilage defects. A primary candidate close to the DDR1 locus is Sickler syndrome. Due to the ubiquitous expression of collagen, other non-hereditary diseases could potentially be linked to DDR1 and DDR2 misfunction as well.

### Example 3

The following are the details for the experiments discusssed in Example 2 demonstrating that members of the collagen family are ligands for DDR's.

Experimental Procedures

Reagents, Cell Lines and Plasmids

Matrigel was obtained from Collaborative Biomedical Products (Bedford, Mass.). All types of collagen and other reagents were purchased from Sigma (St. Louis, Mo.). Human embryonic kidney fibroblast 293 cells, human mammary carcinoma T-47D cells, and human fibrosarcoma HT 1080 cells were obtained from American Tissue Culture Collection and cultivated under the recommended conditions. The expression of the Shc PTB domain as a bacterial glutathione-S-transferase fusion protein has been published previously (van der Geer et al., 1996). The DDR expression vectors have been described earlier (1). Parts of the extracellular domains of DDR1 (amino acids 29-186) and DDR2 (amino acids 28-367) were cloned into pET30a vector (Novagen, Madison, Wis.) in frame with a His-tag and expressed in E. coli under the T7 promoter. Proteins were purified by Ni-affinity chromatography (Qiagen) and used to raise antibodies. The peptide ALLLSNPAYRLLLATYARC was used to raise antibodies against the b-isoform of DDR1 (amino acids 505-523). Antibodies to DDR1 (amino acids 894-913) and insulin receptor (amino acids 1365-1382) were purchased from Santa Cruz, Inc. (Santa Cruz, Calif.). Monoclonal antiphosphotyrosine antibody 4G10 was from Upstate Biotechnology, Inc. (Lake Placid, N.Y.).

Purification of Collagen

Adult mice were sacrificed and the collagen-containing tendon was dissected from their tails using sterile forceps. The tendon was incubated in 500 mM acetic acid on a shaker at 4° C. overnight. Non-soluble material was removed by centrifugation. Soluble material was dialyzed against 10 mM acetic acid. Purity and integrity of collagen was evaluated by SDS-PAGE.

Collagen type IV was extracted from matrigel with a buffer containing 2 M guanidinium hydrochloride, 50 mM Tris(pH 7.5), 2 mM DTT (Kleinman et al., 1982). Soluble material was dialyzed against 500 mM acetic acid. Alternatively, matrigel was extracted with 500 mM acetic acid, 1% pepsin and the soluble collagen dialyzed against 500 mM acetic acid (Timpl et al., 1979).

Transient Expression in 293 cells and Western Blot Analysis

Semiconfluent 293 cells were transfected by calcium-phosphate precipitation with a cytomegalovirus-based expression vector. Sixteen hours later, cells were transferred to serum-free media for another 24 h. Cells were stimulated with 10 μg/ml collagen for 90 min and lysed with 1% Triton-X 100, 50 mM HEPES (pH 7.5), 150 mM NaCl, 1.5 mM MgCl₂, 5 mM EGTA, 5 mM EDTA, 10% glycerol, 10 mM NaF, 1 mM phenylmethylsulfonyl fluoride (PMSF), 1 mM Na-orthovanadate, 10 μg/ml aprotinin. The cellular lysates were centrifuged 10 min at 4° C. and 13,000 rpm and aliquots of the supernatant were subjected to SDS-PAGE or further analyzed by immunoprecipitation with specific antibodies for 3 h at 4° C. on a rotating wheel. The immunocomplex was washed three times with 20 mM HEPES (pH 7.5), 150 mM NaCl, 0.1% Triton, 10% glycerol and analyzed by SDS-PAGE. Proteins were transferred to nitrocellulose membrane (Schleicher & Schuell) and immunoblotted with antibodies diluted 1:500 in 50 mM Tris (pH 7.5), 150 mM NaCl, 5 mM EDTA, 0.05% gelatin overnight. Western blots were developed using horseradish peroxidase-coupled secondary antibody (Biorad) and enhanced chemiluminescence (Amersham). For reprobing, the membrane was stripped in 70 mM Tris (pH 6.8), 2% SDS, 0.1% P-mercaptoethanol at 55° C. for 30 min. For in vitro kinase assays, immunoprecipitates were washed twice with in 40 mM HEPES (pH 7.5), 20 mM MgCl₂, 2 mM MnCl₂, 100 μM orthovanadate, 5 μM dATP and incubated with 5 μCi [γ-³²P] ATP at 30° C. for 20 min. The products of the kinase reaction were monitored by SDS-PAGE and autoradiography.

Binding Analysis

100 μg of mouse collagen type I were iodinated by the N-chloro-benzenesulfonamide method using 1 mCi of Na[¹²⁵I] (NEN) and one iodo-bead (Pierce). Labeled collagen was recovered by Sephadex G50 (Pharmacia) chromatography and was found to have a specific activity of 5×10⁶ cpm/μg. Binding to DDR receptors was measured by transfecting 293 cells on 24 well plates with expression plasmids for DDR1a, DDR1b or control plasmid. Cells were washed with ice cold PBS supplemented with 1% BSA and 10 mM glucose and were incubated on ice with increasing concentrations of radiolabeled collagen for 2 h. Cells were washed three times with binding buffer and lysed with 100 μl 200 mM NaOH, 1% SDS. The lysates were neutralized with 200 mM HCl and counted in a gamma-scintillator (LKB 1282). Each value is the average of three measurements.

Deglycosylation of Collagen

For deglycosylation, mouse collagen type I was incubated with freshly prepared 10 mM sodium m-periodate for 20 min at room temperature in the dark. The excess of periodate was eliminated by adding 20 mM sodium-bisulphite. The collagen was dialyzed against 10 mM acetic acid overnight.

Assay for MMP-1 Expression

The full length cDNA of DDR2 was stabilely expressed in HT 1080 cells using a retroviral expression construct (pLXSN). Neomycin resistant clones were tested for DDR2 expression. Parental and DDR2 overexpressing cells were cultivated in serum free medium and stimulated with 10 μg/ml collagen type I for various periods of time. The conditioned medium was 20-fold concentrated and analyzed for the presence of MMP-1 by Western blotting with the monoclonal antibody 41-IE5 (Oncogene Research Products; Cambridge, Mass.).

Surface Plasmon Resonance

Experiments were done with a BIAcore instrument (Pharmacia). A phosphopeptide corresponding to the NPXY sequence in the polyomavirus middle T antigen (LSLLSNPTpYSVMRSK) was absorbed to the surface of a biosensor chip. Binding of soluble GST-Shc PTB domain fusion protein to the middle T peptide was measured in presence or absence of competing soluble phosphopeptides based on the sequences around tyrosine 513 of DDR1b (ALLLSNPApYRLLLA) or tyrosine 490 of the NGF receptor (HIIENPQpYFSD).

CD Spectroscopy

The CD spectrum of mouse collagen type I was recorded with an AVIV 60DS spectropolarimeter. The melting curve was monitored at 221 nm, while the temperature was increased at a rate of 1° C./min.

Results:

Ligand Activity for DDR1 is Found in Matrigel

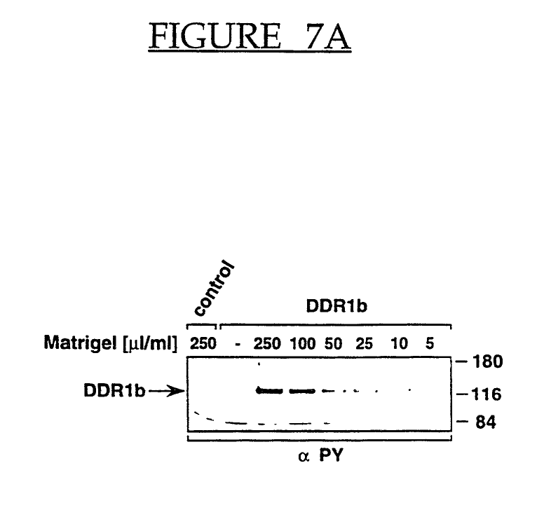

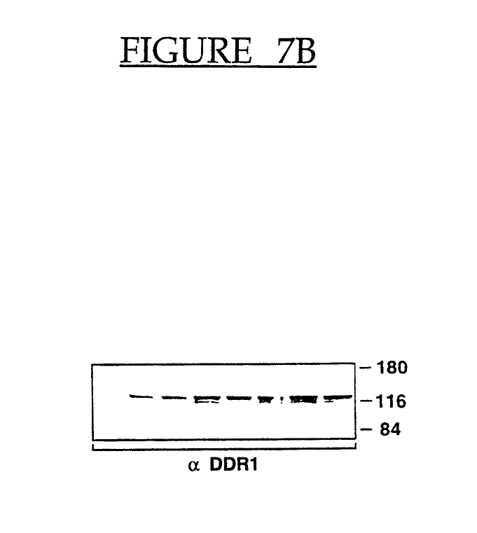

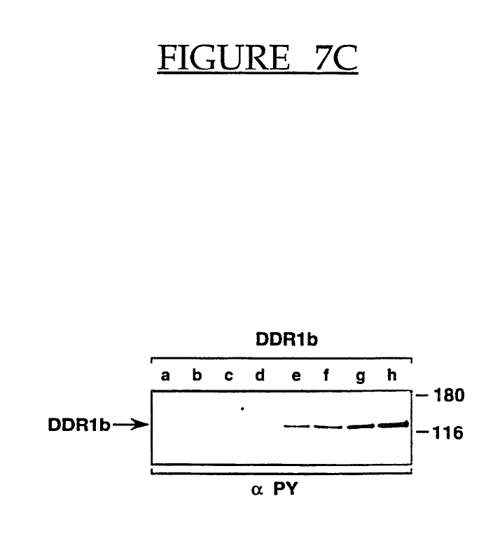

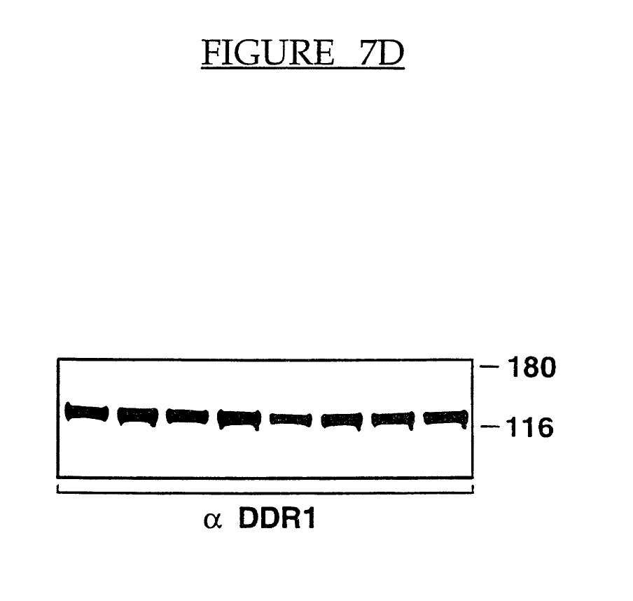

To identify possible sources for the ligand of DDR1, an in vivo screening system was established. Human embryonic kidney fibroblast 293 cells were transiently transfected with expression plasmids coding for DDR1b. Growth-arrested cells were then stimulated with various agents and monitored for receptor activation by anti-phosphotyrosine Western blotting of total cellular lysates. An activity that strongly induced the tyrosine phosphorylation of DDR1b was detected in matrigel, a commercially available preparation of basement membrane proteins from the Engelbreth-Holm-Swarm (EHS) mouse sarcoma. The increase of DDR1b autophosphorylation was dependent on the concentration of matrigel added to the cells (FIG. 7A). Maximal stimulation was observed with 250 μl matrigel per ml tissue culture medium, and an approximately two-fold increase of receptor autophosphorylation was seen with 10 μl matrigel per ml of culture medium.

Matrigel has repeatedly been used as a source for purification and characterization of extracellular matrix proteins. Therefore, the isolated matrix proteins fibronectin, laminin, SPARC, perlecan and collagen were tested for their ability to stimulate DDR1b tyrosine phosphorylation. Of these, neither fibronectin and laminin (FIG. 7C, lanes c and d) nor SPARC and perlecan (data not shown) were able to induce DDR1b autophosphorylation. To test collagen type IV, this matrix protein was isolated from matrigel following the guanidinium hydrochloride extraction protocol of Kleinman et al. (1982) and employed in the in vivo DDR1b autophosphorylation assay. This preparation of purified soluble collagen induced a greater increase in DDR1b tyrosine phosphorylation than did matrigel (FIG. 7C, lane b and e). A similar result was obtained using collagen that was purified from matrigel using an alternative method based on extraction with acetic acid and pepsin digestion (Timpl et al., 1979; FIG. 7C, lane f). Furthermore, commercially available collagen type IV, either from the EHS tumor or from human placenta, induced DDR1b tyrosine phosphorylation to a similar extent as collagen purified from matrigel (FIG. 7C, lanes g and h).

Collagen Induces Tyrosine Phosphorylation of Both DDR Tyrosine Kinase Receptors

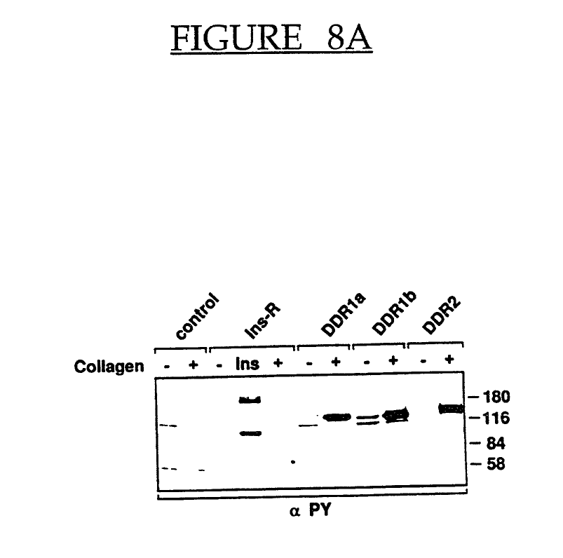

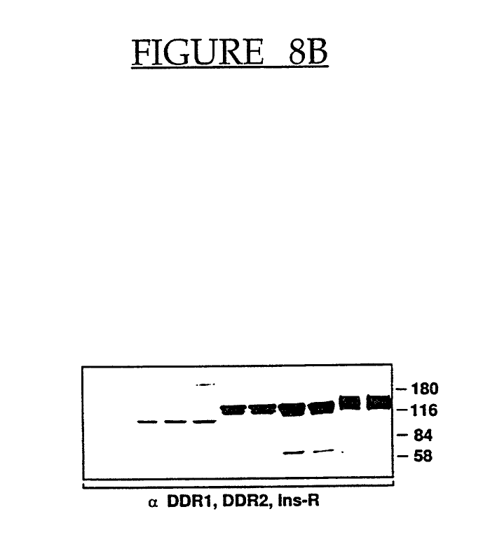

To obtain collagen from a normal tissue, tendon containing collagen type I was mechanically isolated from the tails of adult mice, and the collagen was solubilized by extraction with 500 mM acetic acid. This preparation of collagen showed the same substantial activation of DDR1 tyrosine phosphorylation as collagen type IV isolated from matrigel (FIG. 8A). Using a concentration of 10 μg collagen per ml of tissue culture medium in this assay, purified collagen induced similar levels of tyrosine phosphorylation on both the a- and b-isoform of DDR1 (FIG. 8A). Soluble, purified mouse tail collagen was also used to stimulate 293 cells transfected with a cDNA for DDR2, the second member of the discoidin domain subclass of RTKs. Collagen induced an increase in the tyrosine phosphorylation of DDR2 similar to that observed for DDR1 (FIG. 8A). In contrast, 293 cells transfected with expression plasmids for the insulin-receptor or the EGF-receptor showed no increase in receptor tyrosine phosphorylation after treatment with collagen (FIG. 8 and data not shown).

Kinetics of DDR1 and DDR2 Activation by Collagen

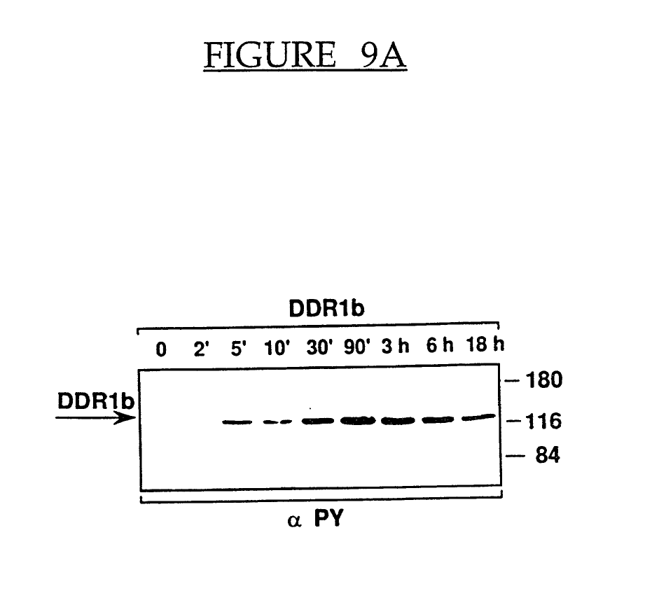

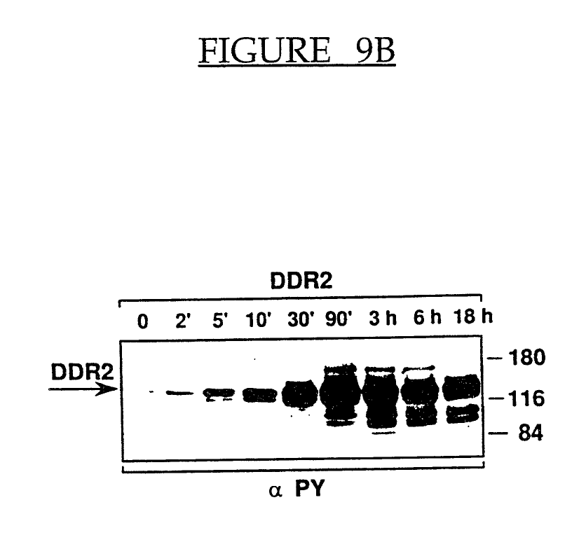

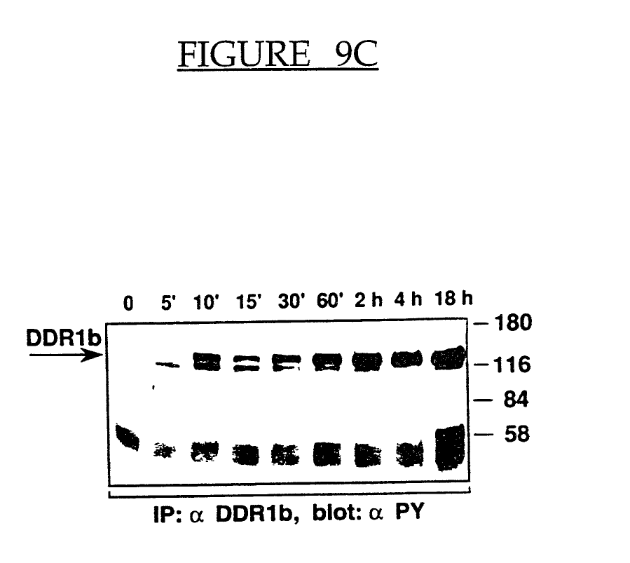

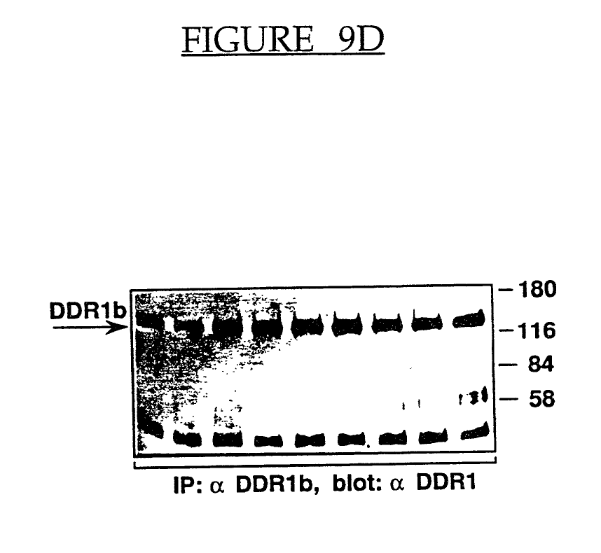

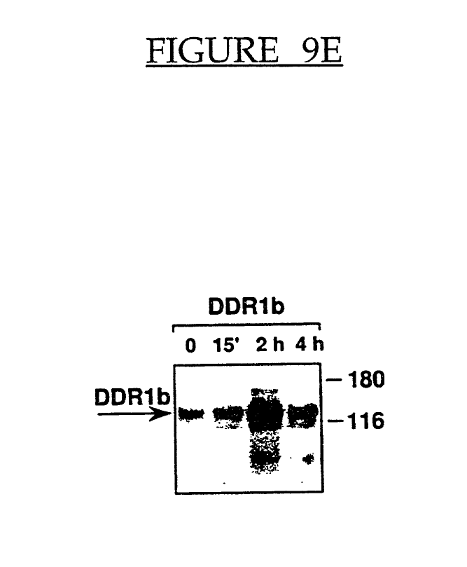

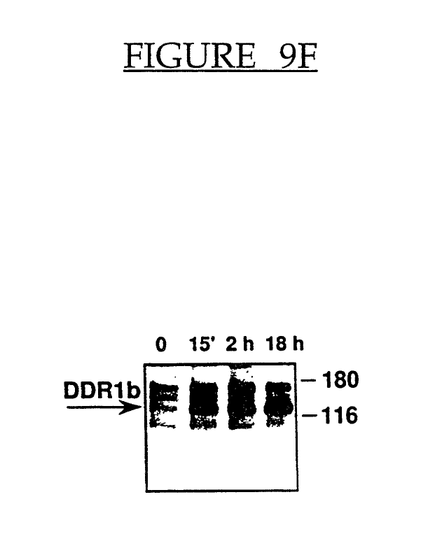

Receptor tyrosine kinases usually become rapidly autophosphorylated upon stimulation by an activating ligand. For example, an increase in autophosphorylation of the receptors for EGF or insulin takes place in a matter of seconds. Maximal activation is generally achieved a few minutes after stimulation and the receptor is then commonly downregulated through a variety of mechanisms, including receptor internalization and proteolysis. However, there are exceptions to this rule; for example, the activation of Eph family receptors by their cell surface ligands is a more protracted affair, requiring at least an hour for maximal receptor phosphorylation (Gale et al., 1996; Holland et al., 1997). The kinetics of DDR1 and DDR2 activation by collagen was investigated using transfected 293 cells, and no significant increase was found in autophosphorylation after 2 min of stimulation with 10 μg/ml (app. 30 nM) mouse collagen type I (FIG. 9A and B). Receptor activation was rather delayed and peaked 90 min to 2 h after stimulation. The tyrosine phosphorylation of both DDR1 and DDR2 was sustained for up to 18 hours following addition of soluble collagen (FIG. 9A and B). The DDR1a and DDR1b isoforms showed identical kinetics of activation.

To investigate the effects of collagen on cells that express endogenous DDR tyrosine kinases, the mammary carcinoma cell line T-47D was employed. Previous results have shown that DDR1 is highly expressed in human carcinoma cell lines, in particular, the breast cancer cell lines BT-20, MDA-MB-175 and T-47D (1) (Perez et al., 1996). As shown in FIG. 9C, DDR1b was inducibly phosphorylated on tyrosine following incubation of T-47D cells with collagen. The time course of endogenous DDR1b tyrosine phosphorylation in T-47D cells was even slower than in 293 cells, with maximal activation being achieved only after stimulation for 18 hours.

To analyze the effect of collagen on DDR1b autokinase activity, the receptor was immunoprecipitated from overexpressing 293 or T-47D cells after various periods of stimulation. The immunoprecipitates were subjected to in vitro kinase reactions and the incorporation of [³²P]-phosphate into the receptor was monitored by autoradiography. In each case, the extent of in vitro receptor kinase activity reflected the state of in vivo tyrosine phosphorylation (FIG. 9E and F). Whereas DDR1b, that was overexpressed in 293 cells, reached maximal in vitro autokinase activity after stimulation for 2 h, endogenous DDR1b from T-47D showed the highest activity after overnight incubation with collagen.

Differential Activation of DDRs by Various Types of Collagen

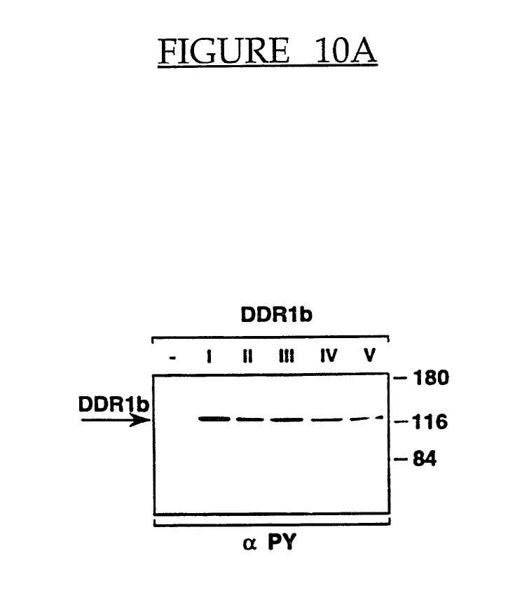

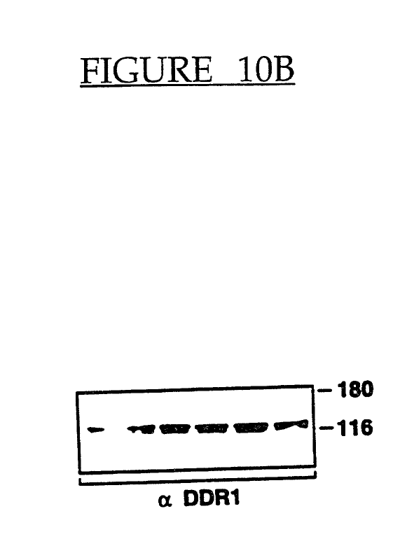

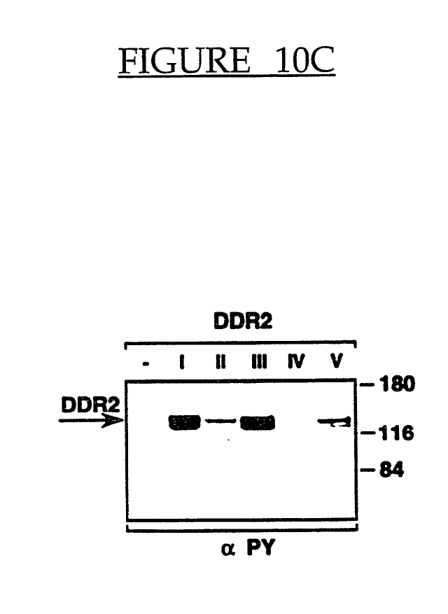

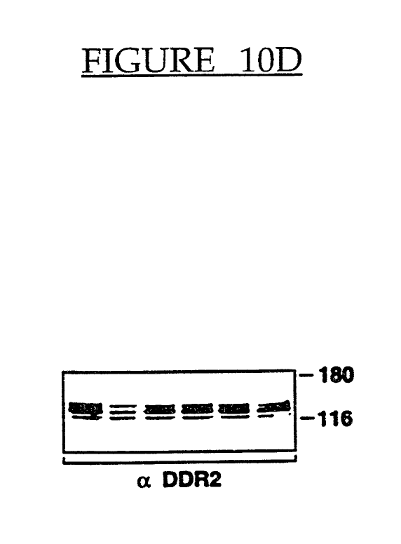

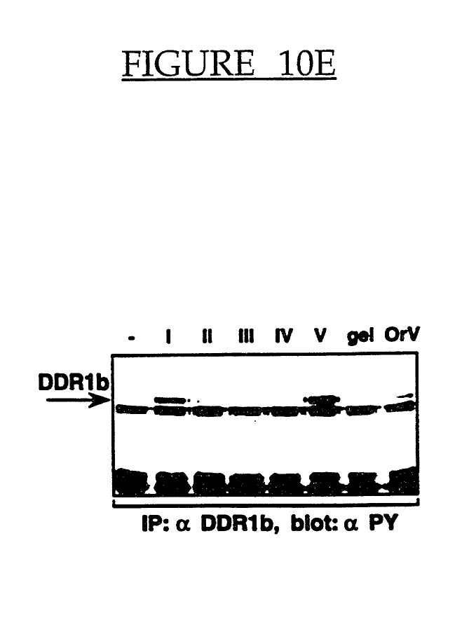

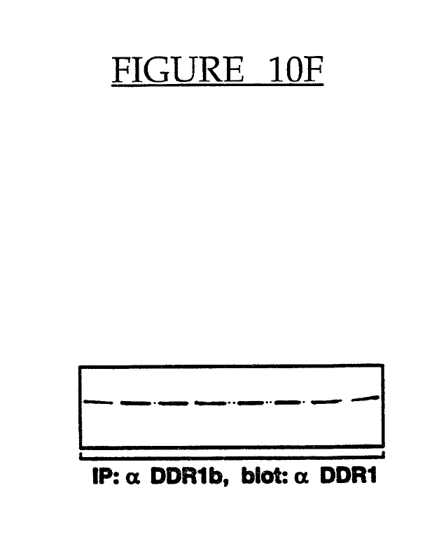

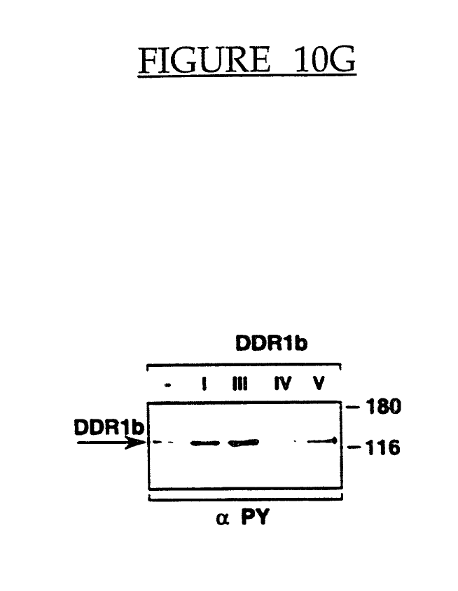

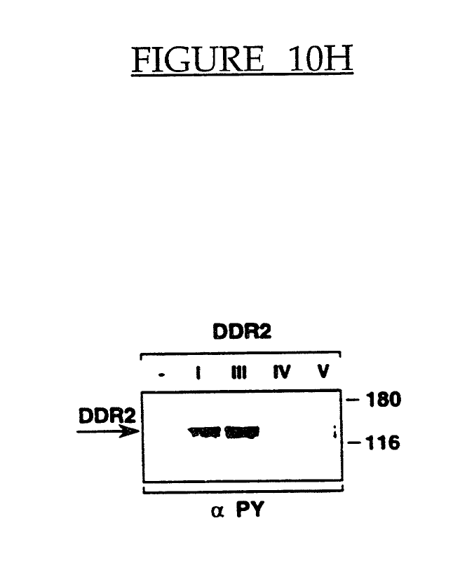

To further investigate the role of different types of collagen in the activation of DDR1 and DDR2, collagen types I, II, III and V purified from human placenta and collagen type II from bovine tracheal cartilage were obtained. To test their ability to induce receptor tyrosine phosphorylation, 293 cells overexpressing DDR1 or DDR2 were employed. After stimulation with 10 μg/ml collagen for 90 min, DDR1 showed an increase in tyrosine phosphorylation with all types of collagen tested (FIG. 10A). In contrast, DDR2 was highly activated only by collagen types I and III, while collagen types II and V-showed moderate activity (FIG. 10C). Surprisingly, collagen type IV, which was originally identified as the ligand activity for DDR1 in matrigel, was not able to stimulate DDR2 tyrosine phosphorylation. The various types of collagen were also tested for their ability to stimulate tyrosine phosphorylation of endogenous DDR1b present in T-47D cells. For this purpose, DDR1b was immunoprecipitated from lysates of stimulated T-47D cells and analyzed by Western blotting with anti-phosphotyrosine antibodies. As shown in FIG. 10E, collagen types I and V gave rise to a substantial increase of DDR1b tyrosine phosphorylation. The level of activation induced by collagen types II, III and IV was lower, but still detectable compared to unstimulated cells. As previously shown, DDR1 tyrosine phosphorylation could also be activated by treating cells with 1 mM orthovanadate, an inhibitor of tyrosine phosphatases. Furthermore, gelatin which contains heat denatured collagen was tested for its capacity to induce DDR1b tyrosine phosphorylation. At a concentration of 10 μg/ml, gelatin did not induce an increase in DDR1b tyrosine phosphorylation, indicating that the native structure of collagen is essential for DDR1 receptor activation in the cell-based assay (FIG. 1E).

Tyrosine Phosphorylation of DDR1 and DDR2 can be Activated by Immobilized Collagen

The experiments described above employed soluble collagen, whereas collagen encountered in vivo will commonly be immobilized as part of the extracellular matrix. The self-assembly of solubilized collagen into fibrils is a spontaneous process that can be induced in vitro by neutralization or evaporation of the solvent. To this end, tissue culture dishes were coated with collagen types I, III, IV and V. Transfected 293 cells that overexpress DDR1b or DDR2 were then resuspended in PBS without trypsin/EDTA treatment and added to the collagen-coated dishes for 90 min. Analysis of receptor phosphorylation indicated that DDR1b became partially tyrosine phosphorylated upon detaching cells from the plate; however receptor tyrosine phosphorylation was further increased after plating on collagen types I, III and V (FIG. 10G). In contrast, no initial receptor phosphorylation was seen after detachment of DDR2 overexpressing cells; DDR2 tyrosine phosphorylation was triggered by plating cells on collagen types I and III, and to a smaller extent on type V (FIG. 10H). Immobilized collagen type IV was inactive in this assay, as also observed for soluble collagen type IV (FIG. 10C).

The ligand Activity for DDR Activation is Pepsin-Resistant, but Collagenase-Sensitive

The preceding data suggest that collagen may stimulate the tyrosine phosphorylation of DDR1 and DDR2. To investigate whether collagen is indeed the molecule responsible for activating these receptors, advantage was taken of the unique structural properties of the members of the collagen family. The primary amino acid sequence of collagen contains stretches of Gly-X-Y repeats that vary in length. By virtue of the glycine residue at every third position, three identical or highly similar polypeptide chains are able to coil around each other, forming a left-handed helix. The residues X or Y are frequently proline or 4-hydroxyproline respectively, which allows further stabilization of the triple helix due to restrictions in chain flexibility and the formation of interchain hydrogen bonds. This particular structure makes collagen resistant to protease cleavage, for example by pepsin, which cleaves after large hydrophobic residues (phenylalanine, methionine, leucine, tryptophan) that are not found in the triple helix of collagen. In contrast, collagenase isolated from Clostridium histolyticum specifically cleaves before every second or third glycine residue of the Gly-X-Y repeat.

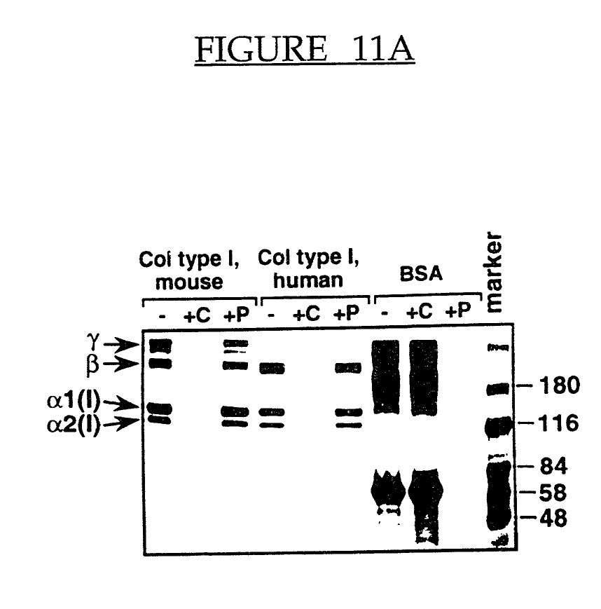

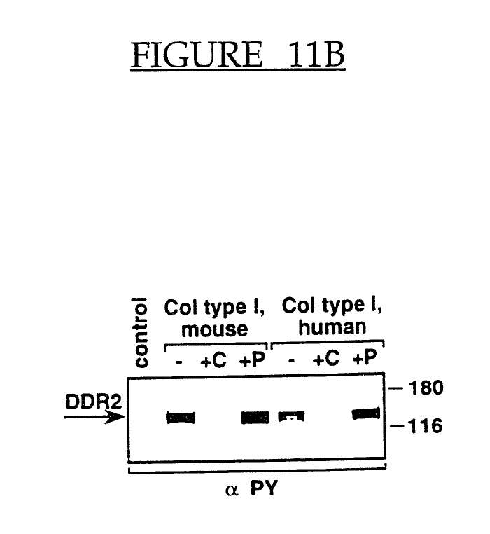

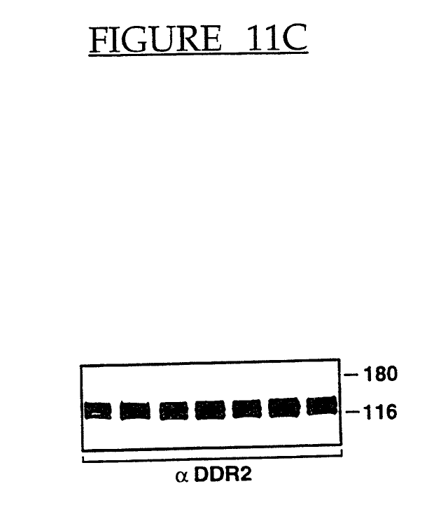

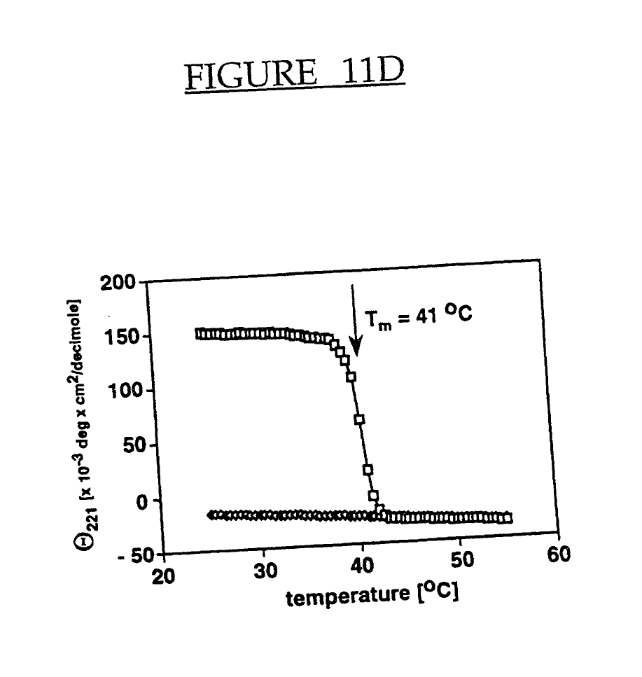

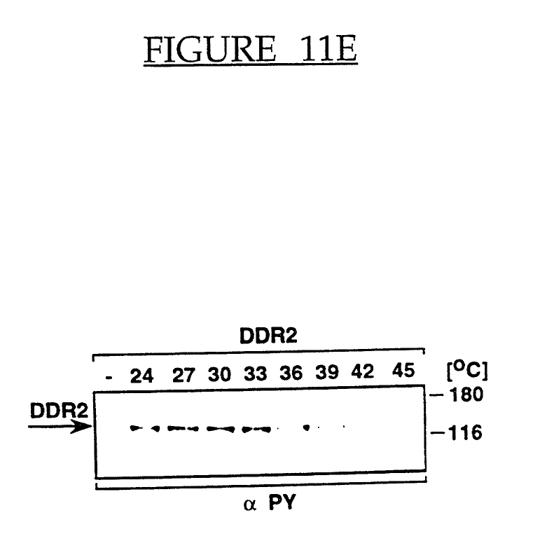

Collagen type I isolated from mouse tail or human placenta was preincubated with pepsin or collagenase for 30 min at 37° C. Following protease treatment, one aliquot was analyzed by SDS-PAGE and staining with Coomassie Blue, while a second aliquot was used to stimulate DDR2 overexpressing 293 cells, which were subsequently analyzed for DDR2 tyrosine phosphorylation. As shown in FIG. 11A, mouse and human collagen type I were effectively digested by collagenase, but were resistant to the proteolytic activity of pepsin. In contrast, bovine serum albumin was not degraded by collagenase, but was digested by pepsin treatment. Analysis of stimulated cells showed that collagenase treatment abrogated the ability of collagen to induce DDR2 tyrosine phosphorylation, whereas pepsin-treated collagen retained its stimulatory activity (FIG. 11B). This result indicates that the ability of the collagen preparation to activate DDR2 depends on the integrity of collagen. The finding that the stimulatory activity is sensitive to the specific proteolytic enzyme collagenase, but resistant to the more general protease pepsin, supports the observation that the induction of DDR tyrosine phosphorylation is due to collagen itself rather than a distinct protein non-covalently associated with collagen.

Thermal Denaturation of Collagen Inhibits the Stimulatory Activity for DDR Tyrosine Phosphorylation

As outlined above, the triple helical structure of collagen is stabilized by hydrogen bonds. At higher temperatures, the polypeptide chains of collagen will therefore melt and finally denature into a random coil. To pursue the possibility that the ligand activity for DDR receptors is intrinsic to collagen, mouse collagen type I was subjected to thermal denaturation and the circular dichroism spectrum at a wavelength of 221 nm was recorded. The absorption dropped sharply at 40° C. (FIG. 11D, squares), and the heat treatment resulted in the irreversible denaturation of collagen (FIG. 11D, diamonds). The thermal melting transition midpoint was calculated to be 41° C. To test the effect of thermal denaturation on the ability of the collagen preparation to stimulate DDR2 tyrosine phosphorylation, aliquots of collagen type I were incubated at various temperatures between 27° C. and 45° C. for 30 min and these samples were then incubated with DDR2 overexpressing in 293 cells. As shown in FIG. 11E, the ligand activity of collagen preparation was markedly reduced after heat-treatment at 39° C. and almost completely abolished at temperatures above 42° C.

These data demonstrate that the activity of the collagen preparation in stimulating DDR2 kinase activity decreases over the same temperature range as the trimeric structure of collagen is unfolded due to the melting of the coiled coil. Therefore, the triple helical configuration of collagen is essential for full activation of the DDR receptors in this assay.

The Interaction Between DDR and Collagen is Direct

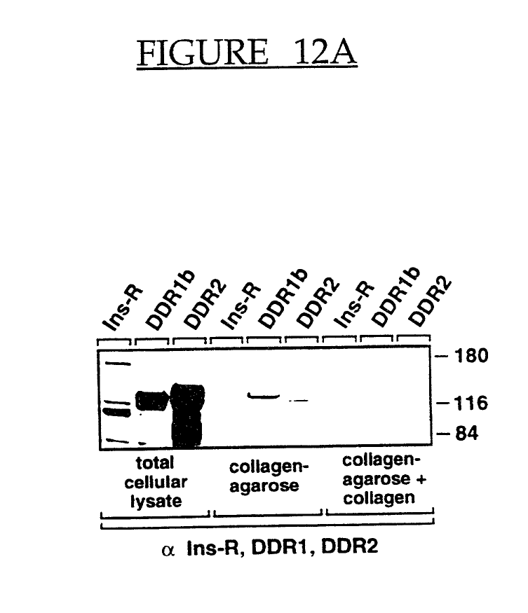

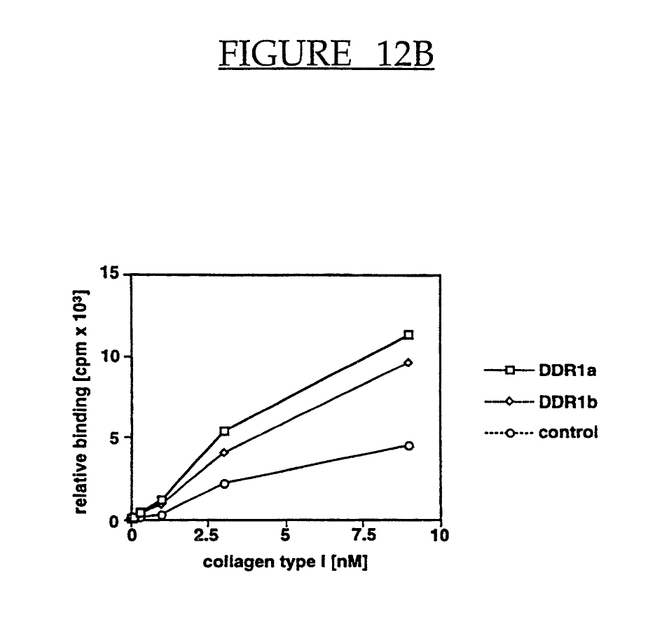

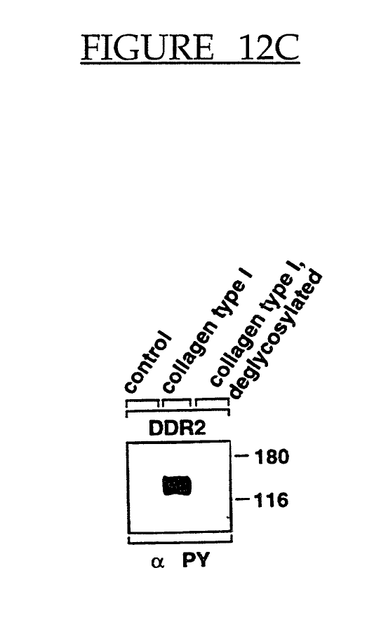

The ability of collagen to stimulate DDR tyrosine phosphorylation could be due to a direct association of collagen with the extracellular domain of the receptor, or could represent an indirect effect of collagen, for example on clustering of cell surface molecules. To explore whether collagen might associate directly and specifically with DDR receptors, collagen covalently linked to agarose beads was employed, in an in vitro mixing experiment. Equal amounts of cellular lysates from 293 cells overexpressing DDR1, DDR2 or insulin receptor were incubated with collagen-agarose in the absence or presence of soluble collagen type 1. As shown in FIG. 12A, collagen-agarose bound to DDR1 and DDR2, but not to the insulin receptor. This interaction of immobilized collagen with DDR1 or DDR2 was competed by an excess of soluble collagen.

Consistent with the view that collagen interacts directly with DDR receptors, the ability of collagen to induce DDR tyrosine phosphorylation was shown to be abrogated by a lysine to alanine point mutation in the DDR receptor cDNA that destroys receptor kinase activity, but was not inhibited by treatment of cells with cycloheximide (data not shown). These results suggest that collagen directly induces DDR autophosphorylation, and does not act through induction of a distinct DDR ligand.

Expression of DDR1 and DDR2 Increases Cell Surface Binding Sites for Collagen

To investigate whether expression of DDR tyrosine kinases increases the amount of cell surface receptors for collagen, mouse collagen type I was iodinated and incubated in varying concentrations with 293 cells that have been transfected with DDR1a, DDR1b or a control plasmid. As shown in FIG. 12B, binding of collagen was approximately three times higher to DDR1 transfected cells than to control cells. The binding of ¹²⁵I-collagen to DDR1 was almost fully competed with a 100 fold excess of cold ligand (data not shown).

The Interaction Between DDR2 and Collagen is Sensitive to the Carbohydrate-Moiety of Collagen

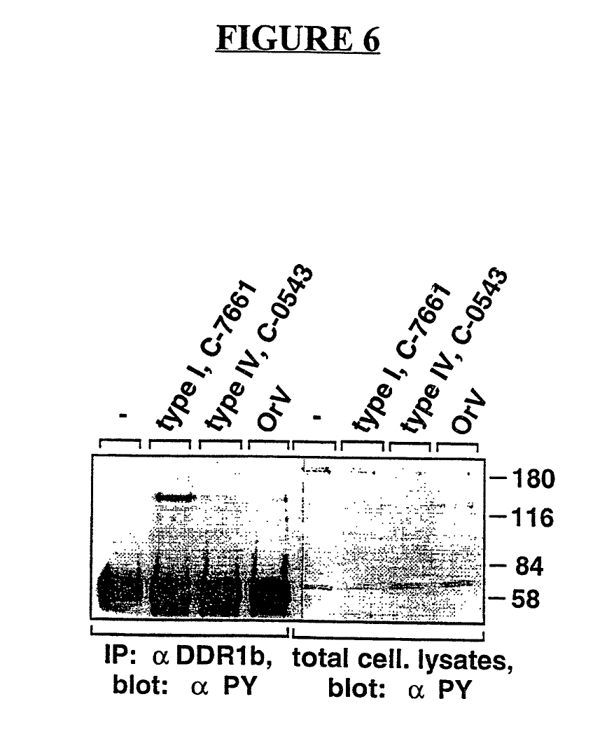

Multiple studies have shown that collagen is extensively glycosylated through N- and O-linked carbohydrates after its initial synthesis (for review see Kivirikko and Myllylä, 1982). In particular, several lysines are oxidized to hydroxylysine and afterwards linked to galactose and glucose. The monosaccharide composition of mouse tail collagen was analyzed using fluorophore-assisted carbohydrate eiectrophoresis technology (Higgins & Friedman, 1995) and it was found that this collagen preparation contained high amounts of glucose and galactose, smaller amounts of fucose and mannose, but no sialic acid, N-acetyl-glucosamine or N-acetyl-galactosamine (data not shown). Therefore, collagen was treated with sodium m-periodate to partially remove the glyco-conjugate. The periodate-treatment of collagen type I did not induce hydrolysis of the polypeptide backbone or denature the collagen triple helix (data not shown). In contrast, the ability of collagen to stimulate DDR2 in 293 overexpressing cells was significantly reduced after deglycosylation (FIG. 6C). Therefore, either the N- or O-linked glyco-fraction of collagen (or both) may be important for DDR2 activation. Occassional commercial preparations of collagen have been encountered that do not give DDR receptor activation. This may be explained by a loss of native conformation or by a failure of a modification such as glycosylation.

The She PTB Domain Binds to DDR1b after Collagen Stimulation

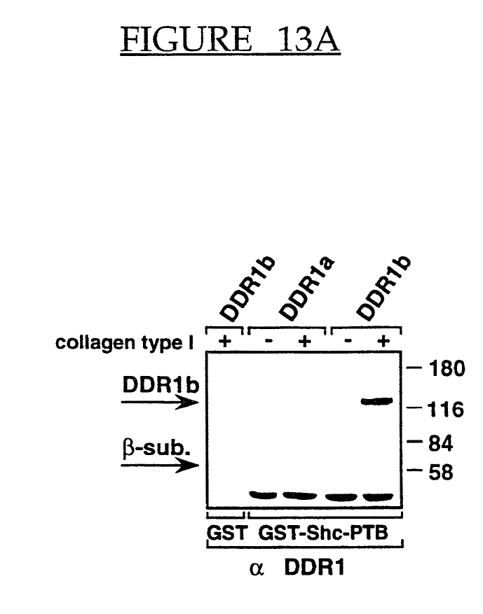

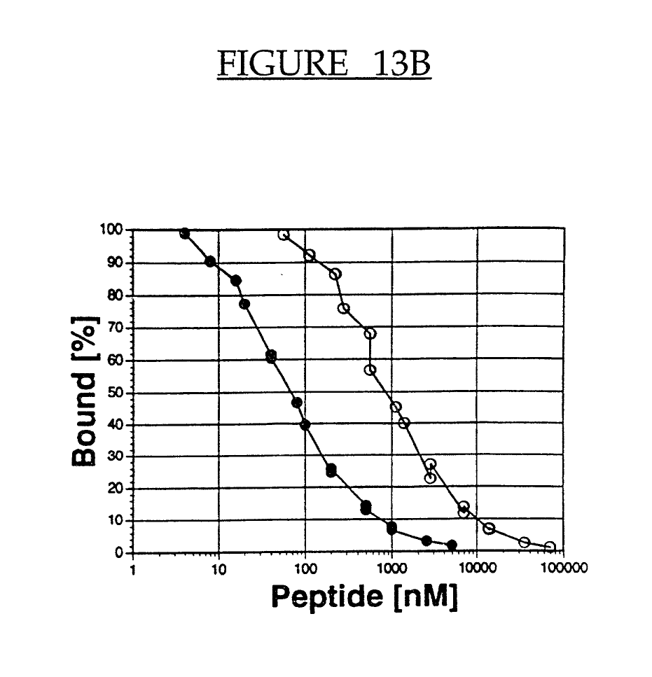

Since collagen induces DDR1 tyrosine phosphorylation, it is possible that such receptor autophosphorylation might create binding sites for phosphotyrosine recognition modules, such as SH2 or PTB domains. In this regard, it is of interest that the juxtamembrane insert of DDR1b contains the motif LSNPAY (including tyrosine 513), which corresponds to the consensus binding motif for the Shc PTB domain. The Shc PTB domain binds with high affinity to phosphotyrosine-containing peptides with the sequence ψXNPXpY (where ψ is a hydrophobic residue)(23)(van der Geer et al., 1996). The possibility that Shc might interact with autophosphorylated DDR1b was tested. Indeed, DDR1b bound to a GST-fusion protein containing the Shc PTB domain after collagen stimulation (FIG. 13A). The Shc PTB domain did not bind to either the a-isoform of DDR1 or DDR2, which lack the XNPXY motif found in DDR1b. This demonstrates that collagen-stimulation of DDR1 induces receptor autophosphorylation and consequent formation of docking sites for modular downstream signaling molecules.

To quantify the interaction between DDR1b and Shc more precisely, surface plasmon resonance technology was used in which a soluble phosphopeptide derived from the juxtamembrane region of DDR1b (residues 503-518) was employed to inhibit binding of the GST-Shc PTB fusion protein to a polyomavirus middle T antigen phosphopeptide, immobilized on a biosensor chip. As shown in FIG. 13B, a concentration of approximately 800 nM DDR1b phosphopeptide, containing the sequence around tyrosine 513, inhibited binding of the Shc PTB domain to the middle T antigen phosphopeptide by 50% (IC₅₀). These findings contrast with an IC₅₀ value of 70 nM found when a phosphopeptide around the NPXY motif of the NGF receptor was used in a comparable experiment (FIG. 13B), but are similar to the IC₅₀ of the middle T antigen phosphopeptide itself.

Activation of DDR2 Induces Expression of Matrix Metalloproteinase-1

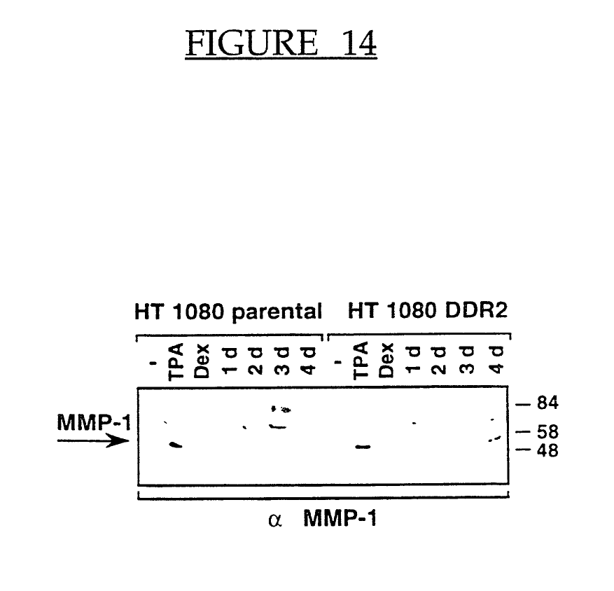

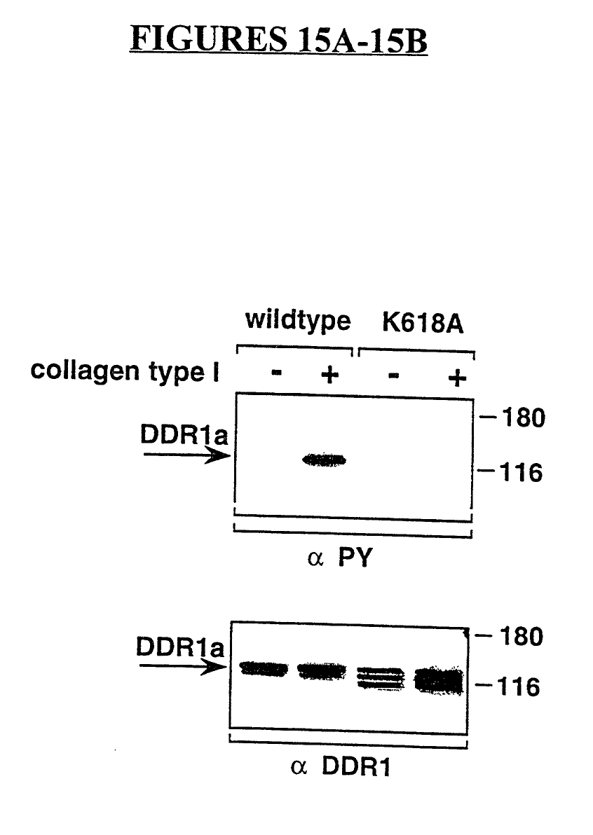

Extracellular matrix degradation and remodelling is largely controlled by the activity of matrix metalloproteinases (MMPs). To test whether DDR signaling influences the expression of MMPs, DDR2 was stably expressed in the human fibrosarcoma cell line HT 1080, which shows no detectable expression of DDR receptors. Parental and DDR2 overexpressing HT 1080 cells were stimulated for various periods of time with 10 μg/ml collagen type I. The amount of matrix metalloproteinase-1 (MMP-1) secreted by the cells was measured by Western blot analysis of the conditioned media. As shown in FIG. 14, the expression of MMP-1 was upregulated in HT 1080 cells overexpressing DDR2 after stimulation with collagen for 4 days. In contrast, MMP-1 was not induced in parental HT 1080 in response to collagen. MMP-1 expression was induced in both, parental and DDR2 overexpressing cells after treatment with phorbol 12-myristate 13-acetate (TPA), an activator of MMP expression. These data suggest that DDR receptor activation results in secretion of coliagenolytic activity, which might ultimately lead to extracellular matrix breakdown.

Discussion

Collagen Activation of Discoidin Domain Receptors

A search for ligands of the DDR subfamily of receptor tyrosine kinases has unexpectedly revealed that collagen, one of the most abundant proteins in vertebrates, is able to bind and to activate both DDR receptors. The analysis showed that activation of overexpressed DDR1 is triggered by all five collagens tested, whereas DDR2 is only activated by collagen types I and III, and to a lesser extent by collagen types II and V. In a human mammary carcinoma cell line endogenous DDR1 is strongly triggered by collagen I and V, and to a smaller extent by II, III and IV. Therefore, there is some specificity in the interactions of DDR1 and DDR2 with collagen molecules.

Collagen types I, II, III, V and XI have an uninterrupted Gly-X-Y repeat that spans more than 1000 amino acids and forms a perfect triple-helical structure. Individual helices polymerize thereby generating fibers with high tensile strength. In contrast, collagen type IV is characterized by approximately 20 short interruptions of the triple-helix, which provide more flexibility and allow the formation of network-like structures (Prockop & Kivirikko, 1995). Collagen type IV is the main component of the basement membrane surrounding various tissues and organs.

Three lines of evidence support the conclusion that the binding epitopes for DDR1 and DDR2 are located in the Gly-X-Y repeat region and that the triple-helical conformation is essential for receptor activation and autophosphorylation. Firstly, treatment of collagen with pepsin, a protease that cleaves collagen only in the non-helical regions at the N- and C-terminus, did not affect its ligand activity. In contrast, bacterial collagenase, which specifically digests collagen in the Gly-X-Y repeat region, abolished receptor activation. Second, the ability of collagen preparations to activate DDR receptors was sensitive to heat denaturation. The collagen triple-helix is mainly held together by non-covalent linkages, making it sensitive to thermal denaturation. Various types of collagen become denatured in the range between 37° C. and 45° C., at which temperature the triple-helical fold is irreversibly destroyed (Niyibizi et al., 1984). The DDR stimulating activity of collagen preparations is markedly decreased at the thermal melting transition midpoint of collagen, and that gelatin has neither in vitro binding activity, nor the ability to induce tyrosine phosphorylation in vivo. Thirdly, although denatured collagen is unable to activate DDR receptors, collagen type I that was denatured in 7 M urea and thereafter refolded by dialysis into a physiological solvent recovered its ability to induce tyrosine phosphorylation of DDR1 and DDR2 (data not shown). Several collagen-associated molecules, such as chondroitin-sulphate A, B and C, decorin and heparin were tested and no effect on DDR activity ws found. The results discussed above indicate that collagen itself, or a very tightly linked molecule, acts as a stimulatory ligand for DDR receptors

Biological Functions of Proteins with Discoidin Motifs

The discoidin domains of DDR1 and DDR2 have extensive homology (approximately 75%) to the discoidin proteins of Dictyostelium discoideum. In the slime mold, discoidins are expressed and secreted during the formation of the slug and the fruiting body, and function as lectins by binding to N-acetyl-galactosamine and galactose (Rosen et al. 1973). In discoidin-l deficient Dictyostelium, cells lose their ability to adhere and migrate on the substratum, resulting in a defect in ordered cell aggregation (Springer et al., 1984). The activation of DDR receptors by collagen clearly requires the triple-helical peptide backbone of collagen, but may also involve N- or O-linked carbohydrate moieties, as treatment of collagen with periodate resulted both in partial deglycosylation and marked reduction of ligand activity. If the capacity to bind carbohydrates is conserved in the discoidin domains of mammalian DDR, it is possible that DDR1 and DDR2 recognize mono- or oligosaccarides bound to collagen. Further specificity and high affinity could be provided by the triple-helical conformation of the peptide backbone close to the glycosylation site, which could also allow the oligomerization and consequent transphosphorylation of bound DDR tyrosine kinases. In contrast to DDR2, however, periodate treatment did not affect the ability of collagen to activate DDR1 (data not shown).

Collagen as a Signaling Molecule

The observation that DDR receptors bind a protein as abundant as collagen raises a puzzling issue which is fundamental to cell surface signaling receptors for matrix components, namely how cytoplasmic signaling is regulated. The activation of DDR kinases by collagen follows a very delayed time course relative to conventional growth factor receptors, consistent with the possibility that DDR receptors monitor the relationship of the cell to the extracellular matrix rather than mediating an acute signaling response. However, since DDR1 receptor isolated from mouse embryos or adult brain contains little phosphotyrosine (Perez et al., 1996), there must be regulatory mechanisms that control DDR1 activation. Although DDR1 RNA expression has been detected in the outer epithelial layer of the lung, kidney and colon in close proximity to the basement membrane (1), it is possible that the localization of DDR receptors to specific subregions of the cell surface might be regulated. Furthermore, the signaling activity of DDR1 can potentially be controlled by inclusion or exclusion of the juxtamembrane binding site for the Shc PTB domain. It is interesting, in this regard, that DDR1 is the first example of a RTK whose docking sites for downstream targets are directly controlled by alternative splicing.

Shc is apparently recruited to the al I integrin complex, and may play a role in cell survival and proliferation upon engagement of this integrin (Wary et al., 1996). Since the Shc PTB domain is bound to phosphorylated tyrosine 513 in the DDR1b-isoform, it is possible that stimulated DDR1 and activated integrin receptors converge on the same signaling pathways. However, triggering of the MAP kinase pathway by has not been shown suggesting that Shc fulfills another function in DDR1 signaling. In this context, it is interesting that Shc SH2 domain binds the phosphorylated tail of cadherin, a transmembrane cell-cell adhesion receptor (Xu et al., 1997), raising the possibility that Shc might bridge distinct adhesion molecules through its PTB and SH2 domains. Shc may also couple to cytoplasmic signaling pathways other than the MAP kinase pathway.

In embryogenesis, the appropriate expression of different types of collagen induces the correct formation of bone and cartilage (Mundlos & Olsen, 1997). A number of human genetic diseases caused either by aberrant expression of collagen or point mutations in the primary collagen sequence, result in skeletal malformations or inherited osteoporosis (Prockop & Kivirikko, 1995). The early expression of DDR receptors in embryogenesis indicates that they may have a role in the patterning of cartilage and bone formation.

Interactions with collagen are also potentially important for controlling cell shape and movement, for example in the movement and joining of epithelial sheets during development. Recent studies have shown a strikingly high level of DDR1 and DDR2 in various human primary tumors (1). In particular, fast growing tumors, originating from mammary, ovarian and lung epithelial cells, have elevated expression of DDR1. These tumors are characterized by their invasive growth into neighboring tissues and organs, leading to tumor cell metastasis. The initial stimuli necessary to induce breakdown of the matrix barrier and migration of cells away from the tumor are largely unknown (Alves et al., 1995b). Elevated expression of matrix metalloproteinases, enzymes that specifically degrade collagens and elastin, has been found in various solid tumors, and therefore links this class of enzymes to tumor growth and metastasis (Stetler-Stevenson et al., 1996). For example, elevated levels of MMP-1 were found to be associated with poor prognosis in colorectal cancer (Murray et al., 1996). Because DDR1 and DDR2 are triggered by collagen, and because activated DDR2 promotes MMP-1 expression, the two receptors may have a role in tumor cell activation and subsequent degradation of the matrix by metalloproteinases. One model places the DDR receptors as sensors for collagen, as a major component of the extracellular matrix, on the surface of tumor cells. After ligand binding and receptor activation, the DDR signal induces expression and secretion of MMP-1, which in turn degrades the collagen molecules surrounding the tumor allowing tumor cells to migrate and to metastasize. In normal cells, such as keratinocytes, MMP-1 expression is highly elevated after collagen type I stimulation raising the possibility that DDR receptor activity could be involved in wound healing (Sudbeck et al., 1994; 1997).

The slime mold is a simple model for metazoan development, as it exists in the form of unicellular amoebae that can inducibly aggregate into multicellular structures that develop two distinct cell types: spore and stalk cells. Recent evidence has indicated that an SH2 domain signaling pathway, involving tyrosine phosphorylation of a Dictostelium Stat protein, plays a key role in mediating differentiation of these two cells types in response to the morphogen DIF (Kawata et al., 1997). Here, the data establish that an extracellular domain originally identified in the slime mold lectin discoidin, and known to be important for the aggregation of differentiating cells, is coupled in vertebrates to tyrosine kinase signaling activated by collagen, and is potentially important in the ordered movement of normal mammalian cells, and the disordered migration of tumor cells.

### Example 4

DDR1a with K618A mutation is no longer activated by collagen.

The experiment illustrated in FIG. 15A and 15B shows that tyrosine phosphorylation of DDR1a is clearly dependent on an intact catalytic domain. The activation of DDR1a by collagen is abolished by an inactivating (dominant negative) mutation in the catalytic domain. No other kinases seem to be involved in the in vivo tyrosine phosphorylation of DDR1a in response to collagen.

Blocking Antibodies to α1- or β1-Integrins Do Not Inhibit the Activation of DDR1.

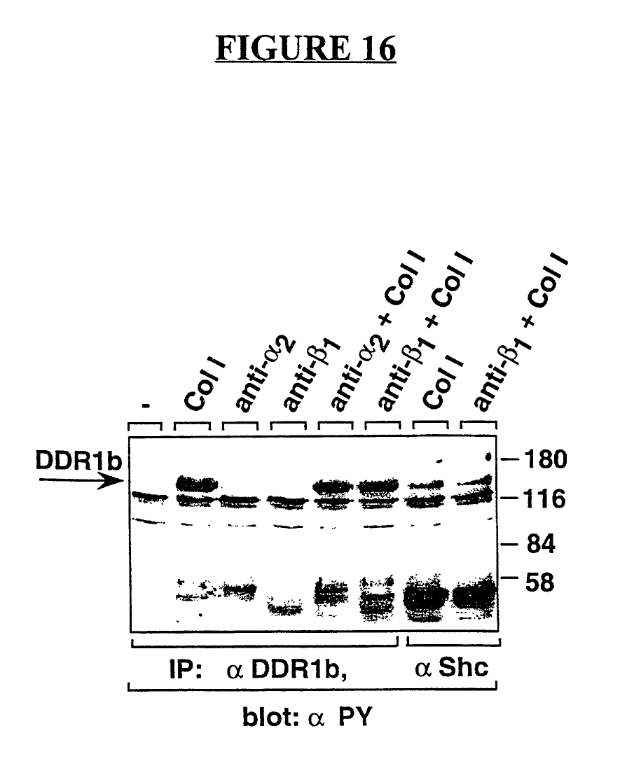

Integrins of the type α1β1 and α2β1 have long been known to be receptors for collagen. Therefore, tests were conducted to determine if these integrins are somehow involved in the activation of DDR1b. The mammary carcinoma cell line T-47D, which endogenously expresses the b-isoform of DDR1, was used. Monoclonal antibodies directed against the extracellular domains of integrins can block binding to collagen and therefore signaling of integrins. T-47D cells were treated with antibody A2-IIE10 against α2-integrin and antibody DE9 against β1-integrin (both from Upstate Biotechnology) in the absence or presence of 10 μg/ml collagen type I overnight. DDR1b or Shc were immunoprecipitated from cellular lysates and analyzed by Western blotting with antiphosphotyrosine antibody. The experiment illustrated in FIG. 16 shows that activation of integrins is not necessary for DDR1b activation. The extent of DDR1b tyrosine phosphorylation after stimulation with collagen in T-47D cells with blocked integrin receptors is identical to untreated cells

The Binding of DDR1b to She is Also Not Altered After Blocking Integrin Signalling.

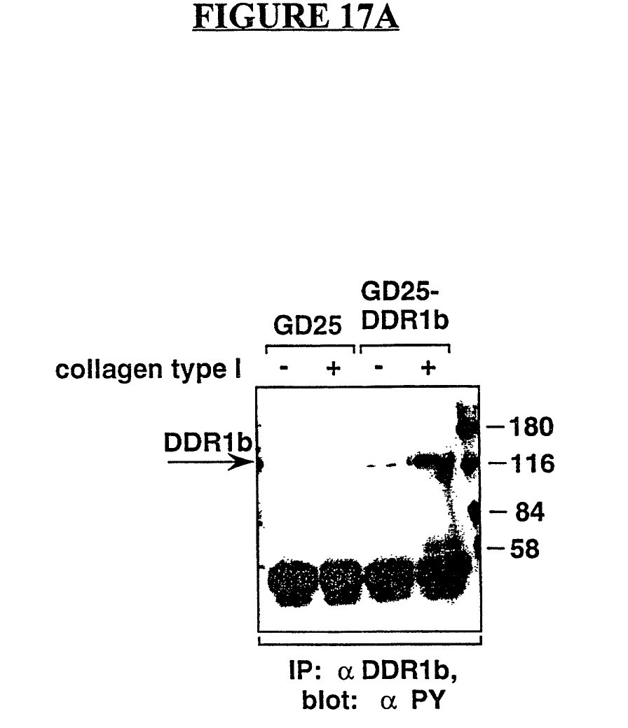

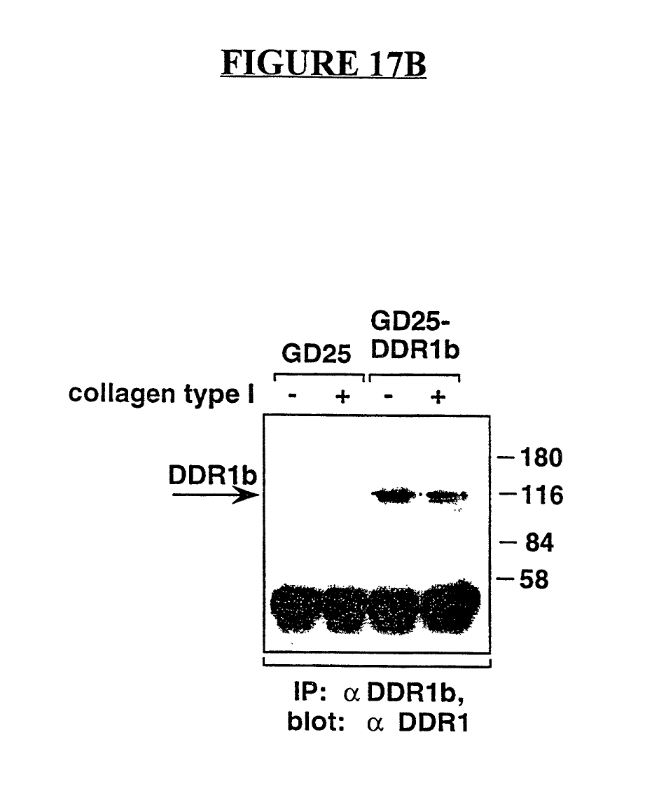

DDR1 is activated by collagen in integrin β1-deficient cells. the signalling of DDR1b in the cell line GD25 (Dr. R. Fassler, Martinsried, Germany), which is derived from integrin β1-knockout mice, was tested. In these cells, a functional integrin receptor for collagen is absent. The cDNA coding for DDR1b was transfected into GD25 cells using a retroviral transfer protocol. DDR1b overexpressing and parental cells were stimulated with collagen type I overnight. DDR1b was immunoprecipitated from cellular lysates and analysed by Western blotting with antiphosphotyrosine antibodies (FIG. 17A). The blot was reprobed with antibodies against DDR1 (FIG. 17B). Using a genetically modified cell line, the experiment illustrated in FIGS 17A and 17B shows that DDR1b can signal in the absence of the two integrin-type collagen receptors.

Slow Activation of DDR1b in Integrin β1-Deficient Cells.

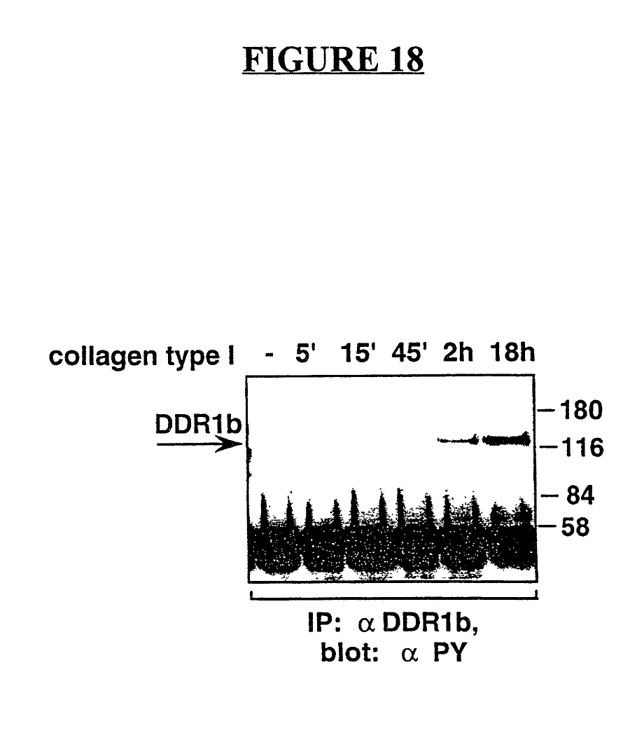

The generation of DDR1b overexpressing GD25 cells is described in FIGS. 17A and 17B. These cells were stimulated with collagen type I for various periods of time. Immunoprecipitated DDR1b was analysed in a Western blot with antiphosphotyrosine antibody. The results shown in FIG. 18 indicate that DDR1b activation in integrin β1-deficient cells is as slow as in normal cells, indicating that the protracted activation of DDR1b is not due to the action of integrins. Activation of DDR1 and DDR2 receptor does not influence EGF mediated MAPK activation.

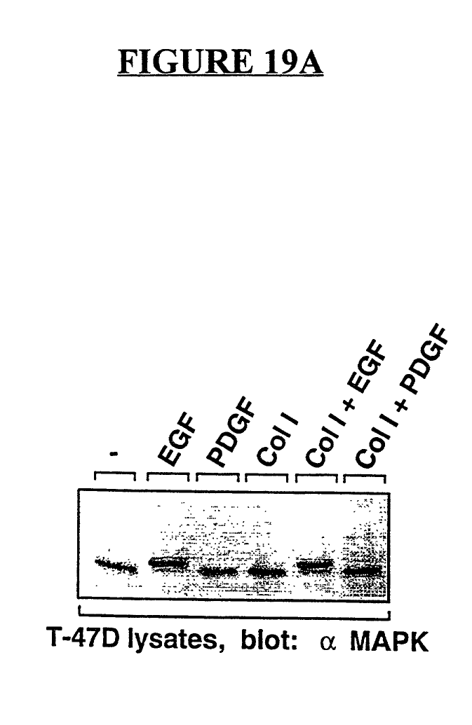

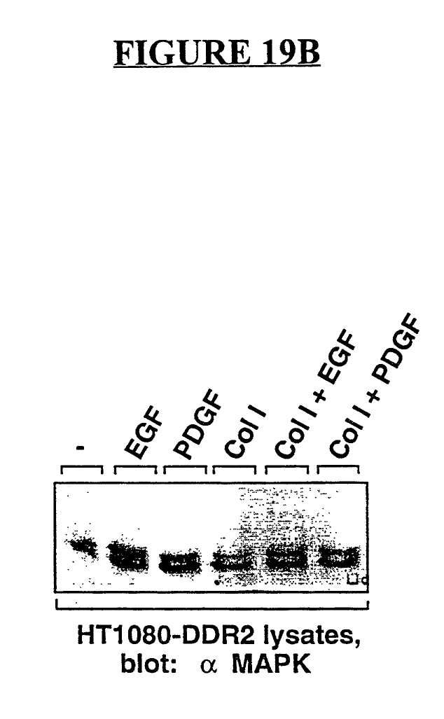

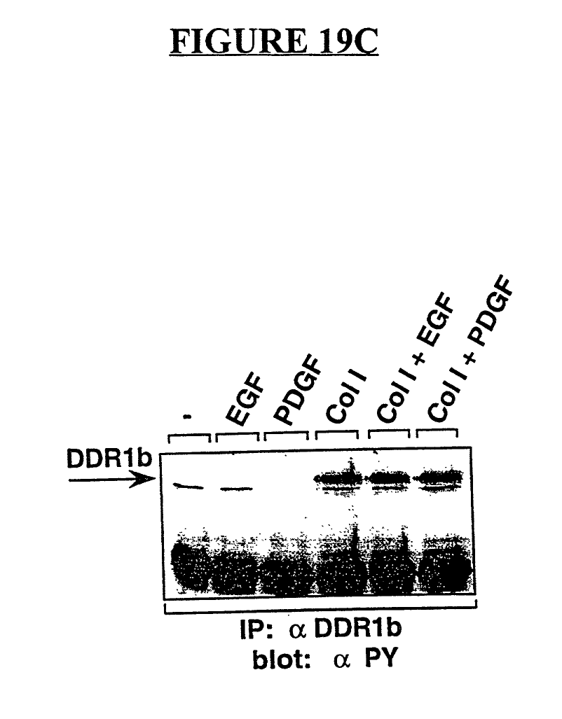

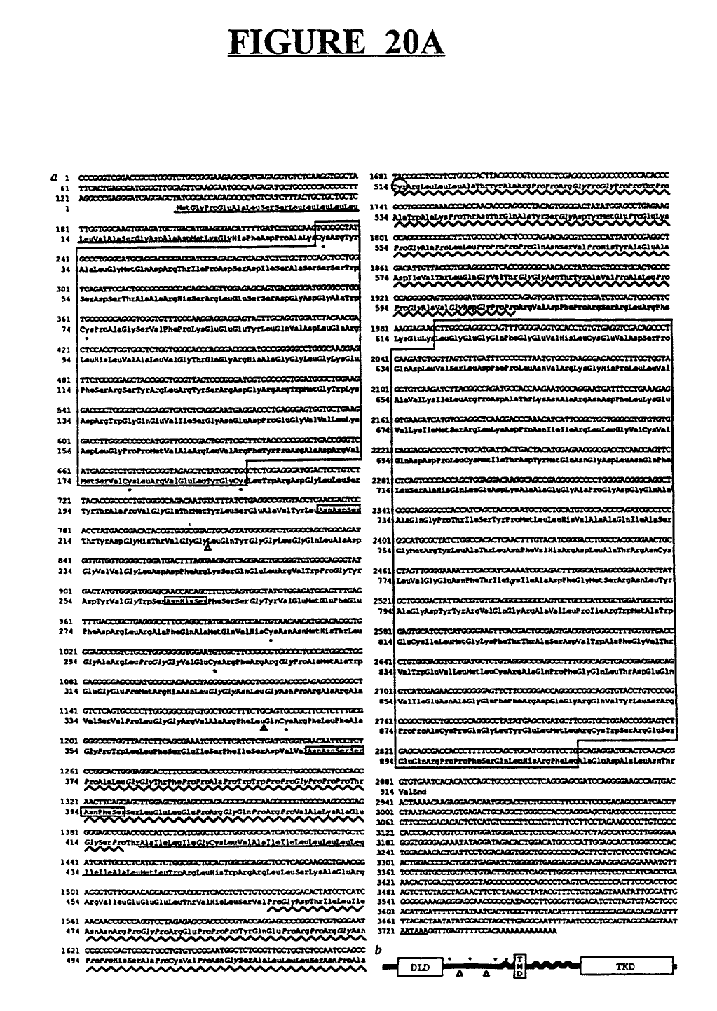

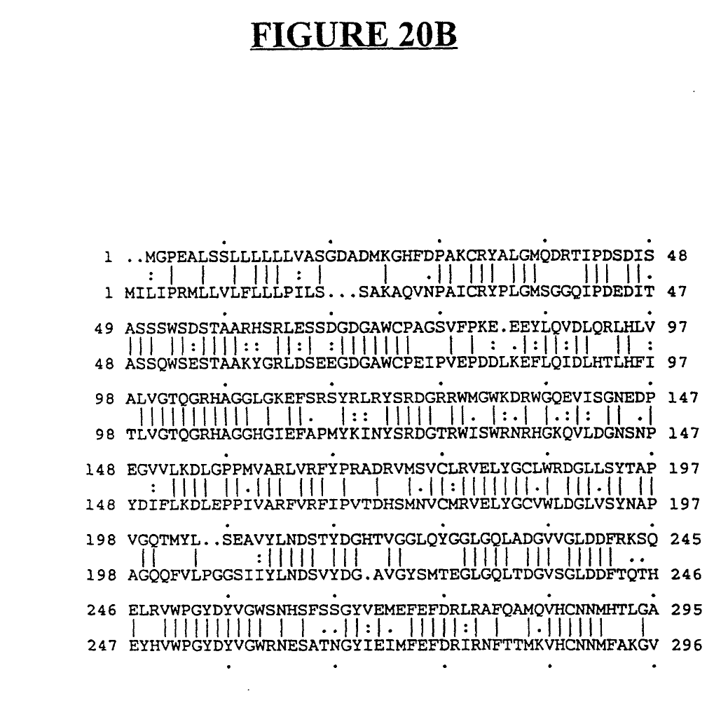

T-47D or HT 1080 overexpressing DDR2 cells were stimulated with PDGF or EGF for 5 min, with collagen type I overnight and with a combination of EGF/collagen or PDGF/collagen. Aliquotes of cellular lysates were separated by SDS-PAGE and probed with an antibody to MAPK (FIG. 19A (T-47D) and FIG. 19B (HT 1080-DDR2). Activated MAPK shows slower migration on SDS-PAGE than non-activated. MAPK becomes activated by EGF or EGF/collagen treatment, but not by PDGF, collagen or PDGF/collagen treatment The remaining lysates from T-47D cells were used to immunoprecipitate DDR1b. Western blotting with antiphosphotyrosine antibodies show, that DDR1b is activated by collagen and not by EGF or PDGF (FIG. 19C). The combination of collagen with EGF does not decrease the extent of MAPK activation or the tyrosine phosphorylation of DDR1b. The experiments illustrated in FIGS. 19A to 19C demonstrate that DDR1 and DDR2 activation does not result in activation of the MAPK pathway. Furthermore, activation of MAPK by EGF is not influenced by simultaneous activation of DDR receptors.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

1. Alves, F., Vogel, W., Mossie, K., Millauer, B., Höfler, H., and Ullrich A. (1995) Oncogene 10, 609-618.

2. Johnson, J. D., Edman, J. C., and Rutter, W. J. (1993) Proc. Natl. Acad. Sci. USA 90, 5677-5681

3. Perez, J. L., Shen, X., Finkemagel, S., Sciorra, L., Jenkins, N. A., Gilbert, D. J., Copeland, N. G., and Wong, T. W. (1994) Oncogene 9, 211-219

4. Zerlin, M., Julius, M. A., and Goldfarb, M. (1993) Oncogene 8, 2731-2739

5. Di Marco, E., Cutuli, N., Guerra, L., Cancedda, R., and De Luca, M. (1993) J. Biol. Chem. 268, 24290-24295

6. Sanchez, M. P., Tapley, P., Saini, S. S., He, B., Pulido, D., and Barbacid, M. (1994) Proc. Natl. Acad. Sci. USA 91, 1819-1823

7. Laval, S., Butler, R., Shelling, A. N., Hanby, A. M., Poulsom, R., and Ganesan, T. S. (1994) Cell Growth & Diff. 5, 1171-1183

8. Karn, T., Holtrich, U., Bräuninger, A., Böhme, B., Wolf, G., Rübsamen-Waigmann, H., and Strebhardt, K. (1993) Oncogene 8, 3433-3440

9. Lai, C., and Lemke, G. (1994) Oncogene 9, 877-883

10. Barker, K. T., Martindale, J. E., Mitchell P. J., Kamalati, T., Page, M. J., Phippard, D. J., Dale, T. C., Gusterson, B. A., and Crompton, M. R. (1995) Oncogene 10, 569-575

11. Kavanaugh, W. M., and Williams, L. T. (1994) Science 266, 1862-1865

12. Blaikie, P., Immanuel, D., Wu, J., Li, N., Yainik, V., and Margolis, B. (1994) J. Biol. Chem. 2695 32031-32034

13. van der Geer, P., Wiley, S., Lai, V. K. -M., Olivier, J. P., Gish, G. D., Stephens, R., Kaplan, D., Shoelson, S., and Pawson, T. (1995) Curr. Biol. 5, 404-412

14. Lai, V. K. -M., Olivier, J. P., Gish, G. D., Henkemeyer, M., McGlade, J., and Pawson, T. (1994)Mol.Cell. Biol.15, 4810-4818.

15. Campbell, K. S., Ogris, E., Burke, B., Su, W., Auger, K. R., Druker, B. J., Schaffhausen, B. S., Roberts, T. M., and Pallas, D. C. (1994) Proc. Natl. Acad. Sci. USA 91, 6344-6348

16. van der Geer, P., and Pawson, T. (1995) TiBS 20, 277-280

17. Obermeier, A., Lammers, R., Wiesmüller, K. H., Jung, G., Schlessinger, J., and Ullrich, A. (1993) J. Biol. Chem. 268, 22963-22966

18. Batzer, A. G., Blaikie, P., Nelson, K., Schlessinger, J., and Margolis, B. (1995) Mol. Cell. Biol. 15, 44034409

19. Rozakis-Adcock, M., McGlade, J., Mbamalu, G., Pelicci, G., Daly, R., Thomas, S., Brugge, J., Pelicci, P. G., Sclessinger, J., and Pawson, T. (1992) Nature 360,689-692

20. Damen, J. E., Liu, L., Rosten, P., Humphries, R. K., Jefferson, A. B., Majerus, P. W., and Krystal, G. (1996) Proc. Natl. Acad. Sci. USA (in press)

21. van der Geer, P., Wiley, S., Gish, G. D., Lai, V. K. -M., Stephens, R., White, M. F., Kaplan, D., and Pawson, T. (1996) Proc. Natl. Acad. Sci. USA (in press)

22. Songyang, Z., Margolis, B., Chaudhuri, M., Shoelson, S. E., and Cantley, L. C. (1995) J. Biol. Chem. 270, 14863-14866

23. Kavanaugh, W. M., Turck, C. W., and Williams, L. T. (1995) Science 268, 1177-1179

24. Dikic, I., Batzer, A. G., Blaikie, P., Obermeier, A., Ullrich, A., Schlessinger, J., and Margolis, B. (1995) J. Biol. Chem. 270, 15125-15129

25. Gustafson, T. A., He, W., Craparo, A., Schaub, C. D., and O Neill, T. J. (1995) Mol. Cell. Biol. 15, 2500-2508

26. Zhou, M. -M., Ravichandran, K. S., Olejniczak, E. T., Petros, A. M., Meadows, R. P., Sattler, M., Harlan, J. E., Wade, W. S., Burakoff, S. J., and Fesik, S. W. (1995) Nature 378, 584-592

27. He, W., O Neill, T. J., Gustafson, T. A. (1995) J. Biol. Chem. 270, 2325-23262

28. Wolf, G., Trüb T., Ottinger E., Groninga, L., Lynch, A., White, M. F., Miyazaki, M., Lee, J., and Shoelson, S. E. (1995) J. Biol. Chem. 270, 27407-27410

29. Guiton, M., Gunn-Moore, F. J., Glass, D. J., Geis, D. R., Yancopoulos G. D., and Tavare, J. M. (1995) J. Biol. Chem. 270, 20384-20390

30. Gillespie, L. L., Chen, G., and Paterno, G. D. (1995) J. Biol. Chem. 270, 22758-22763.

31. Alves F., Vogel, W., and Ullrich A. (1995b). Tumorinvasion—Die Rolle der Rezeptoren. In Molekularbiologische Grundlagen der Gastroenterologie, H. G. Beger, M. Manns, H. Greten, eds. (Berlin: Springer), pp. 17-27.

32. Gale, N. W., Holland, S. J., Valenzuela, D. M., Flenniken, A., Pan, L., Ryan, T. E., Henkemeyer, M., Strebhardt, K., Hirai, H., Wilkinson, D. G., Pawson, T., Davis, S., and Yancopoulos, G. D. (1996). Neuron 17, 8-19.

33. Higgins, E., and Friedman Y. (1995). Anal. Biochem. 228, 221-225.

34. Holland, S. J., Gale, N. W., Gish, G. D., Roth, R. A., Songyang, Z., Cantley, L. C., Henkemeyer, M., Yancopoulos, G. D., and Pawson, T. (1997). EMBO J. 16, 3877-3888.

35. Kawata, T., Shevchenko, A., Fukuzawa, M., Jermyn, K. A., Totty, N. F., Zhukovskaya, N. V., Sterling, A. E., Mann, M., and Williams, J. G. (1997). Cell 89, 909-917.

36. Kivirikko, K. I., and Myllylä, R. (1982). Methods Enzymol. 82A, 245-304.

37. Kleinman H. K., McGarvey, M. L., Liotta, L. A., Gehron Robey, P., Tryggvason, K., and Martin, G. R. (1982). Biochemistry 21, 6188-6193.

38. Mundlos, S., and Olsen, B. R. (1997). FASEB J. 11, 227-233.

39. Murray, G. I., Duncan, M. E., O'Neil, P., Melvin, W. T., and Fothergill, J. E. (1996). Nature medicine 2,461-462.

40. Niyibizi, C., Fietzek, P. P., and van der Rest, M. (1984). J. Biol. Chem. 259, 14170-14174.

41. Perez, J. L., Jing, S. Q., and Wong, T. W. (1996) Oncogene 12, 1469-1477.

42. Prockop, D. J. and Kivirikko, K. I. (1995). Annu. Rev. Biochem. 64,403-434.

43. Rosen, S. D., Kafka, J. A., Simoson, D. L., and Barondes, S. H. (1973). Proc. Natl. Acad. Sci. USA 70, 2554-2557.

44. Springer, W. R., Cooper, D. N. W., and Barondes, S. H. (1984). Cell 39, 557-564.

45. Sudbeck, B. D., Parks, W. C., Welgus, H. G., and Pentland, A. P. (1994). J. Biol. Chem. 269, 30022-30029.

46. Sudbeck, B. D., Pilcher, B. K., Welgus, H. G., and Parks, W. C. (1997). J. Biol. Chem. 272, 22103-22110.

47. Stetler-Stevenson, W. G., Hewitt, R., and Corcoran, M. (1996). Sem. Cancer Biol. 7, 147-154.

48. Timpl, R., Bruckner, P., and Fietzek, P. (1979). Eur. J. Biochem. 95, 255-263.

49. van der Geer, P., Wiley, S., Gish, G. D., Lai, V. K. -M., Stephens, R., White, M. F., Kaplan, D., and Pawson, T. (1996). Proc. Natl. Acad. Sci. USA 93, 963-968.

50. Wary, K. K., Mainiero, F., Isakoff, S. J., Marcantonio, E. E., and Giancotti, F. G. (1996). Cell 87,733-743.

51. Xu, Y., Guo, D. -F., Davidson, M., Inagami, T., and Carpenter, G. (1997). J. Biol. 272, 13463-13466.

Detailed Figure Legends

FIGS. 1A to 1E. MCK10b (DDR1b) coimmunoprecipitates with Shc and associates with the Shc PTB domain. FIGS. 1A-1C, Human embryonic kidney fibroblast 293 cells were transfected with expression plasmids encoding MCK10a or MCK10b. After stimulation with 1 mM orthovanadate for 90 min, Shc was immunoprecipitated from cell lysates. After SDS-PAGE the immunoblot was probed with antibodies to phosphotyrosine (FIG. 1A). Subsequently, the blot was stripped and reprobed with antibodies against MCK10 (FIG. 1B), and thereafter against Shc (FIG. 1C). Migration of MCK10b and the three isoforms of Shc, p66, p52 and p46 are indicated. FIG. 1D, Aliquots of the total cell lysates used in (FIG. 1A) were analysed by Western blotting with anti-phosphotyrosine antibodies. The migration of the precursors (prec.) and b-subunits (b-sub.) of MCK10a and MCK10b are indicated. FIG. 1E, GST-fusion proteins containing the Shc SH2 domain or Shc PTB domain were bound to glutathione agarose and incubated with lysates from 293 cells, which had been transfected with MCK10a or MCK10b and stimulated with orthovanadate. Bound proteins were separated by SDS-PAGE, transferred to nitrocellulose and probed with antibodies to MCK10. Molecular weight standards are indicated on the right.

FIGS. 2A and 2B. Phosphorylation of Tyr 513 in the MCK10b (DDR1b) juxtamembrane insert forms a Shc PTB-binding site. FIG. 2A, GST-Shc PTB domain fusion protein bound to glutathione beads was incubated with lysates from MCK10b overexpressing 293 cells in the absence or presence of increasing concentrations of a competing peptide, ALLLSNPApYRLLLA, corresponding to the sequence around tyrosine 513 of MCK10b. Bound protein was detected by immunoblotting with antibodies to MCK10. FIG. 2B, Analysis of the binding of purified GST-Shc PTB domain to the middle T antigen phosphopeptide by surface plasmon resonance in the presence of increasing amounts of MCK10b phosphopeptide (ALLLSNPApYRLLLA, open circles) or NGF receptor phosphopeptide (HIIENPQpYFSD, closed circles), respectively. The percentage of Shc PTB domain bound to the chip surface is plotted against the concentration of competing peptides.

FIGS. 3A to 3F. MCK10b (DDR1b) contains a major autophosphorylation site which is absent from MCK10a (DDR1a). FIGS. 3A and 3B, 293 cells were transfected with plasmids encoding the a- or b-isoform of MCK10 and were in vivo labeled with [32P]-orthophosphate. After stimulation with 1 mM orthovanadate for 90 min, MCK10 was immunoprecipitated and digested with trypsin. The two dimensional tryptic phosphopeptide maps of the a-isoform (FIG. 3A) and b-isoform (FIG. 3B) are shown. FIGS. 3D-3F, MCK10a or MCK10b were immunoprecipitated from transfected cells and subjected to in vitro kinase assays. Labeled protein of the a-isoform (FIG. 3D) and the b-isoform (FIG. 3E) were analysed by tryptic mapping. Equal cpm of MCK10a and MCK10b phosphopeptides are combined and analysed in (FIG. 3F). The origin is indicated by x. FIG. 3C, shematic representation of the phosphopeptides in (FIG. 3B) and (FIG. 3F).

FIG. 4A, 4B and 4C—Transient expression of DDR1a, DDR1b, DDR2, and trkA in 293 cells. Blot total cellular lysates: αPY. Use collagens in 10 μg per ml final concentration.

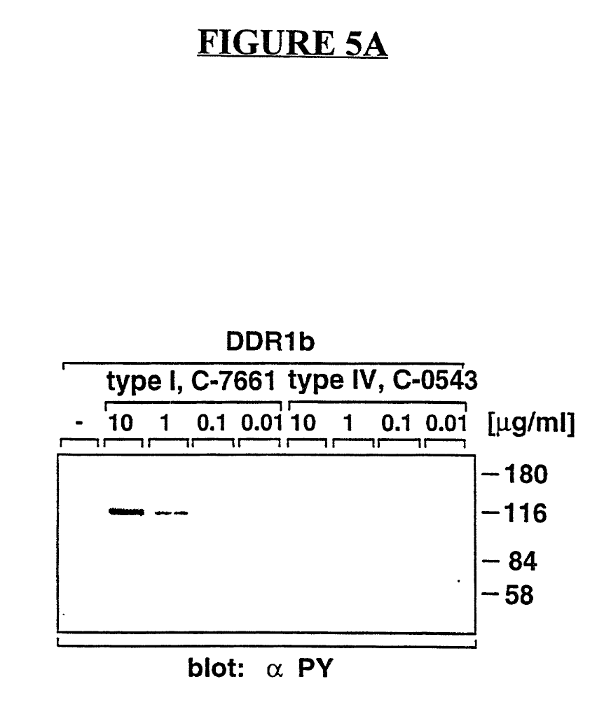

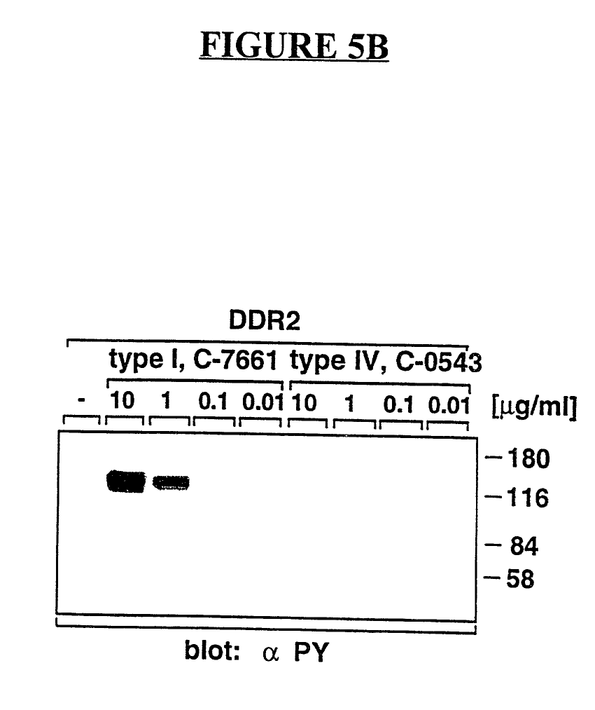

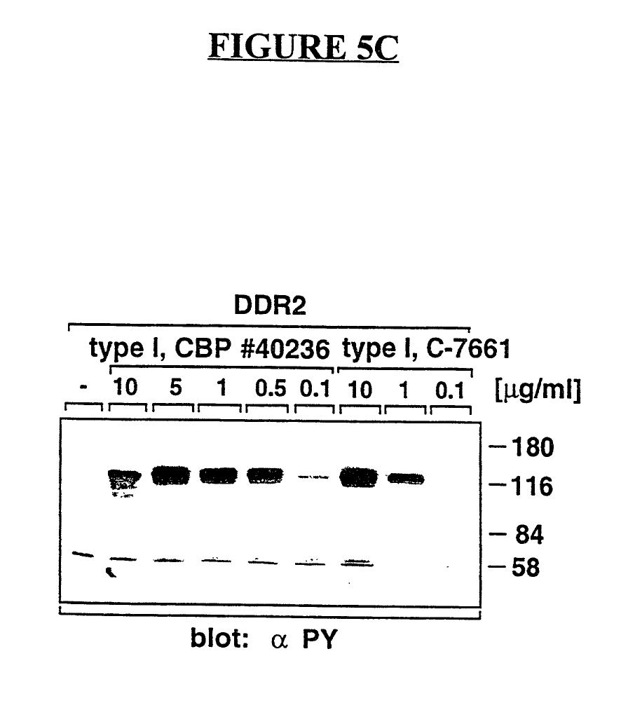

FIG. 5A, 5B, and 5C—α-PY blots of total cellular lysates: Concentration dependent stimulation with Collagen I [C-7661 CBP: Collaborative Biomedical Research] and Collagen IV [C-0543]

FIG. 6—Tyrosine phosphorylation of DDR1b in T-47D, human mammary carcinoma cells. Immunoprecipitation of DDR1b with alternative exon specific antibody, blot PY. Stimulate Collagen I [C-766, Collagen IV [C-0543 for 30 min and 1 mM orthovanadate for 90 min].

FIGS. 7A to D. Identification of Collagen Type IV as the Ligand Activity for DDR1 in Matrigel.

Human kidney fibroblast 293 cells were transfected with a DDR1b expression plasmid. Matrigel was added to the tissue culture medium in the indicated concentrations for 90 min. (FIG. 7A) Cells were lysed and 10 μg total cellular protein was analyzed by SDS-PAGE and Western blotting with antiphosphotyrosine antibody. (FIG. 7B) The blot was stripped and reprobed with antibodies raised against DDR1. (FIG. 7C) 293 cells overexpressing DDR1b were treated with the following reagents: 400 μM acetic acid (a), 50 μl/ml matrigel (b), 10 μg/ml laminin type IV (c), 10 μg/ml fibronectin (d), collagen type IV, partially purified from matrigel by extraction with guanidinium hydrochloride (e) or by extraction with acetic acid and pepsin (f), 10 μg/ml mouse collagen type IV, Sigma C-0543 (g), 10 μg/ml human collagen type IV, Sigma C-5533 (h). Equal amounts of total cellular lysate were probed with antiphosphotyrosine antibody or (FIG. 7D) with anti-DDR1 antibody.

FIGS. 8A and 8B. Mouse Collagen Type I Specifically Activates DDR1 and DDR2.

293 cells were transfected with plasmids coding for human insulin receptor (Ins-R), DDR1a, DDR1b or DDR2. Cells were stimulated with 10 μg/ml mouse collagen type I, 100 nM insulin or left unstimulated. (FIG. 8A) Aliquots of cellular lysates were analyzed by SDS-PAGE and Western blot with antiphosphotyrosine antibody. (FIG. 8B) The blot was reprobed with a mixture of antibodies against DDR1, DDR2 and insulin receptor.

FIGS. 9A to 9F. Delayed Activation of DDR in Response to Collagen.

DDR1b and DDR2 were transfected into 293 cells and were stimulated with 10 g/ml mouse collagen type I for different periods of time. (FIG. 9A and FIG. 9B) Total cellular lysates were probed with antiphosphotyrosine antibody. Maximal activation is seen 90 min after stimulation of DDR1b (FIG. 9A) and DDR2 (FIG. 9B). (FIG. 9C) Human mammary carcinoma T-47D cells were cultivated on 10 cm dishes to confluence and starved in 0.5% serum overnight. Cells were stimulated with collagen type I for various periods of time and lysed. DDR1b was immunoprecipitated from the lysates and analyzed by Western blotting with antiphosphotyrosine antibody. Maximal phosphorylation of DDR1b is seen after stimulation for 18 h. An unidentified, tyrosine phosphorylated protein with an apparent molecular weight of 115 kDa is copurified in the immunoprecipitations. (FIG. 9D) Reprobing the blot with a antibody specific to the C-terminus of DDR1 showed no reactivity with the 115 kDa protein. Prolonged stimulation with collagen increases the processing of DDR1b in T-47D cells, indicated by an additional 62 kDa band. (FIGS. 9E and 9F) DDR1b was immunoprecipitated from overexpressing 293 cells (E) and T-47D (FIG. 9F) cells and subjected to an in vitro kinase reaction. The incorporation of [³²P]-phosphate is monitored by SDS-PAGE followed by autoradiograpy.

FIGS. 10A to 10H. Differential Activation of DDR1 and DDR2 by Various Types of Collagen.

293 cells were transfected with DDR1b or DDR2 and stimulated with 10 μg/ml human collagen types I, III, IV or V and bovine collagen type II. (FIGS. 10A to 10D) Total cellular lysates were probed with antiphosphotyrosine antibody (FIG. 10A: DDR1b and FIG. 10C: DDR2) and reprobed with receptor specific antibodies for DDR1 (FIG. 10B) or DDR2 (FIG. 10D). DDR1b was immunoprecipitated from T-47D cells that had been stimulated for 90 min with collagen types I, II, III, IV, V or gelatin or treated with 1 mM orthovanadate. (FIG. 10E) Immunoprecipitates were analyzed by Western blotting with antiphosphotyrosine antibody and (FIG. 10F) reprobed with DDR1 specific antibody. (FIG. 10G and FIG. 10H) 293 cells that have been transfected with DDR1b or DDR2 were washed off the plates with PBS by repeated pipetting and added to dishes that have been coated with human collagen types I, III, IV and V. After incubation at 37° C. for 90 min, cells were lysed. Total cellular lysates were analyzed by Western blotting with antiphosphotyrosine antibody (FIG. 10G: DDR1b and FIG. 10H: DDR2).

FIGS. 11A to 11E. DDR Ligand Activity is Destroyed by Collagenase-Treatment or Thermal Denaturation.

Collagen type I isolated from mouse or human tissue or BSA were treated with collagenase (from Clostridium histolyticum, 20 ng per μg collagen) or pepsin (from pig mucosa, 2 ng per μg collagen). (FIG. 11A) Equal amounts were analyzed by SDS-PAGE and visualized by Coomassie staining (+C: incubation with collagenase; +P: incubation with pepsin; α1(1) and α2(1): monomeric collagen chains; β and γ: covalently crosslinked oligomeric collagen) or used to stimulated 293 cells overexpressing DDR2. (FIG. 11B) Aliquots of cellular lysates were blotted with antiphosphotyrosine antibody and (FIG. 11C) reprobed with DDR2 specific antibody. (FIG. 11D) Mouse collagen type I (500 ng/ml in 10 mM acetic acid) was melted in a spectropolarimeter and the change in circular dichroism recorded (squares). After heat denaturation, the spectrum was measured again (diamonds). The thermal transition midpoint (Tₘ) is indicated. (FIG. 11E) Aliquots of mouse collagen type I were incubated at various temperatures between 24° C. and 45° C. for 30 min and used to stimulate 293 cells overexpressing DDR2. Total cellular lysates were analyzed by Western blotting with antiphosphotyrosine antibody. FIGS. 12A to 12C. The binding of collagen to DDR1 and DDR2 is direct and activation of DDR2 is reduced after deglycosylation of collagen. (FIG. 12A) Collagen covalently coupled to agarose-beads was incubated with lysates of 293 cells overexpressing insulin-receptor, DDR1b or DDR2 in the absence or presence of 50 g/ml soluble collagen type I. Bound material was analyzed by SDS-PAGE and Western blotting with a mixture of antibodies against insulin-receptor, DDR1 and DDR2 (FIG. 12A). The lower glycosylated isoform of DDR2 showed strongest affinity to collagen-beads. (FIG. 12B) 293 cells transfected with DDR1a (squares), DDR1b (diamonds) or control plasmid (circles) were incubated with various concentrations of iodinated collagen type I and the amount of bound ligand determined by β-counting. (FIG. 12C). Collagen type I was deglycosylated with sodium m-periodate and used to stimulate 293 cells overexpressing DDR2. Total cellular lysates were blotted with antiphosphotyrosine antibody.

FIGS. 13A and 13B. The Adaptor Protein Shc Binds to DDR1b.

(FIG. 13A) The PTB domain of Shc was expressed in E. coli as GST-fusion protein and incubated with lysates from 293 cells overexpressing DDR1a or DDR1b. Bound protein was detected with an antibody against the C-terminus of DDR1. (FIG. 13B) Analysis of the binding of purified GST-Shc PTB domain to the middle T antigen phosphopeptide (LSLLSNPTpYSVMRSK) by surface plasmon resonance in the presence of competing amounts of DDR1b phosphopeptide (ALLLSNPApYRLLLA, open circles) or NGF receptor phosphopeptide (HIIENPQpYFSD, closed circles), respectively. The percentage of Shc PTB domain bound to the chip surface is plotted against the concentration of competing peptide.

FIG. 14. The Activation of DDR2 Induces the Expression of MMP-1.

Parental and DDR2 overexpressing HT 1080 cells were stimulated with collagen type I or TPA for the indicated periods of time. The conditioned media were concentrated and analyzed by Western blotting with antibodies against MMP-1.

FIGS. 15A and 15B: DDR1a with K618A Mutation is No Longer Activated by Collagen.

Sequence alignments showed that lysine 618 in the DDR1a protein is presumably essential for the catalytic function of the tyrosine kinase domain. Therefore, a point mutation was introduced into the cDNA coding for DDR1a, changing lysine 618 to alanine. The mutant cDNA was transiently expressed together with the wildtype DDR1a cDNA in 293 embryonic kidney fibroblast cells. Cells were stimulated with 10 μg/ml collagen type I for 90 min or left untreated. Equal aliquots of total cellular lysate were analyzed by SDS-PAGE and probed with antiphosphotyrosine antibody (FIG. 15A). The blot was reprobed with antibodies to DDR1 (FIG. 15B). This experiment shows that tyrosine phosphorylation of DDR1a is clearly dependent on an intact catalytic domain. The activation of DDR1a by collagen is abolished by an inactivating (dominant negative) mutation in the catalytic domain. No other kinases seem to be involved in the in vivo tyrosine phosphorylation of DDR1a in response to collagen.

FIG. 16: Blocking Antibodies to α1- or β1-integrins Do Not Inhibit the Activation of DDR1.

Integrins of the type α1β1 and α2β1 have long been known to be receptors for collagen. Therefore, the involvement of integrins in the activation of DDR1b was investigated. The mammary carcinoma cell line T-47D, which endogenously expresses the b-isoform of DDR1 was used. Monoclonal antibodies directed against the extracellular domains of integrins can block binding to collagen and therefore signaling of integrins. T-47D cells were treated with antibody A2-IIE10 against α2-integrin and antibody DE9 against β1-integrin (both from Upstate Biotechnology) in the absence or presence of 10 μg/ml collagen type I overnight. DDR1b or Shc were immunoprecipitated from cellular lysates and analyzed by Western blotting with antiphosphotyrosine antibody. This experiment shows that activation of integrins is not necessary for DDR1b activation. The extent of DDR1b tyrosine phosphorylation after stimulation with collagen in T-47D cells with blocked integrin receptors is identical to untreated cells. The binding of DDR1b to Shc is also not altered after blocking integrin signaling.

FIGS. 17A and 17B: DDR1 is activated by collagen in integrin β1-deficient cells. The signaling of DDR1b in the cell line GD25, which is derived from integrin β1-knockout mice, was tested. In these cells, a functional integrin receptor for collagen is absent The cDNA coding for DDR1b was transfected into GD25 cells using a retroviral transfer protocol. DDR1b overexpressing and parental cells were stimulated with collagen type I overnight DDR1b was immunoprecipitated from cellular lysates and analysed by Western blotting with antiphosphotyrosine antibodies (FIG. 17A). The blot was reprobed with antibodies against DDR1 (FIG. 17B). Using a genetically modified cell line, this experiment shows that DDR1b can signal in the absence of the two integrin-type collagen receptors.

FIG. 18: Slow Activation of DDR1b in Integrin β1-Deficient Cells.

The generation of DDR1b overexpressing GD25 cells is described in FIG. 17. These cells were stimulated with collagen type I for various periods of time. Immunoprecipitated DDR1b was analysed in a Western blot with antiphosphotyrosine antibody. This result shows that DDR1b activation in integrin β1-deficient cells is as slow as in normal cells, indicating that the protracted activation of DDR1b is not due to the action of integrins.

FIGS. 19A to 19C: Activation of DDR1 and DDR2 Receptor Does Not Influence EGF Mediated MAPK Activation.

T-47D or HT 1080 overexpressing DDR2 cells were stimulated with PDGF or EGF for 5 min, with collagen type I overnight and with a combination of EGF/collagen or PDGF/collagen. Aliquots of cellular lysates were separated by SDS-PAGE and probed with an antibody to MAPK (FIG. 19A (T-47D) and FIG. 19B (HT 1080-DDR2). Activated MAPK shows slower migration on SDS-PAGE than non-activated. MAPK becomes activated by EGF or EGF/collagen treatment, but not by PDGF, collagen or PDGF/collagen treatment. The remaining lysates from T-47D cells were used to immunoprecipitate DDR1b. Western blotting with antiphosphotyrosine antibodies show, that DDR1b is activated by collagen and not by EGF or PDGF (FIG. 19C). The combination of collagen with EGF does not decrease the extent of MAPK activation or the tyrosine phosphorylation of DDR1b. This experiment demonstrates that DDR1 and DDR2 activation does not result in activation of the MAPK pathway. Furthermore, activation of MAPK by EGF is not influenced by simultaneous activation of DDR receptors.

We claim:

1. An isolated complex comprising (a) a discoidin domain receptor tyrosine kinase or a part thereof, and a collagen or a part thereof; (b) a discoidin domain receptor tyrosine kinase or a part thereof and Shc or PTB binding domain of Shc; or (c) a discoidin domain receptor tyrosine kinase or a part thereof, and a protein containing a PDZ domain, or a PDZ domain.

2. An isolated complex as claimed in claim 1 comprising a discoidin domain receptor 1, or a part thereof, and a collagen type I, II, III, IV, or V, or a part thereof.

3. An isolated complex as claimed in claim 1 comprising a discoidin domain receptor 2, or a part thereof, and a collagen type I or III or a part thereof.

4. An isolated complex as claimed in claim 1 wherein the discoidin domain receptor tyrosine kinase or a part thereof is an oligomer.

5. An isolated complex as claimed in claim 1 comprising a discoidin domain receptor 1b and Shc or PTB binding domain of Shc.

6. A peptide derived from the binding domain of a discoidin domain receptor tyrosine kinase that interacts with a collagen, or interacts with Shc or a protein containing a PDZ domain.

7. A molecule derived from the binding domain of collagen that interacts with a discoidin domain receptor tyrosine kinase.

8. An antibody specific for a complex as claimed in claim 2.

9. A method of modulating a discoidin domain receptor tyrosine kinase-mediated signaling pathway in a cell, comprising reacting a discoidin domain receptor tyrosine kinase protein, or an isoform or a part of the protein, with a collagen or part of a collagen, or reacting the cell with a complex as claimed in claim 1, a peptide as claimed in claim 6, a molecule as claimed in claim 7, or an antibody as claimed in claim 8, thereby modulating the signaling pathway in the cell.

10. A method as claimed in claim 9 wherein the discoidin domain receptor tyrosine kinase protein is a discoidin domain receptor 1, or a part thereof, and the collagen is a type I, II, III, IV, or V collagen, or a part thereof.

11. A method as claimed in claim 9 wherein the discoidin domain receptor tyrosine kinase protein is a discoidin domain receptor 2, or a part thereof, and the collagen is a type I or III collagen, or a part thereof.

12. A method for evaluating a compound for its ability to modulate a DDR-mediated signaling pathway, comprising the steps of:
(a) reacting a collagen, and at least one discoidin domain receptor tyrosine kinase protein, or an isoform or a part of the protein, and a test substance, wherein the collagen and discoidin domain receptor tyrosine kinase protein are selected so that they bind to form a collagen-discoidin domain receptor tyrosine kinase protein complex; and
(b) comparing to a control in the absence of the substance to determine the effect of the substance.

13. A method for identifying a substance which affects a DDR receptor tyrosine kinase-mediated signaling pathway in a cell, comprising (a) reacting a collagen or part thereof, and at least one discoidin domain receptor tyrosine kinase protein, or an isoform or a part of the protein, and a test substance, wherein the collagen and discoidin domain receptor tyrosine kinase protein are selected so that they bind to form a collagen-discoidin domain receptor tyrosine kinase protein complex, under conditions which permit the formation of collagen-discoidin domain receptor tyrosine kinase protein complexes, and (b) assaying for complexes, for free substance, for non-complexed collagen, or for activation of the protein.

14. A method as claimed in claim 13 wherein activation of the protein is assayed by measuring tyrosine phosphorylation of the protein, oligomerization of the protein, binding of a PTB domain to the discoidin domain receptor tyrosine kinase protein juxtamembrane domain, or by assaying for a biological affect on the cell.

15. A method as claimed in claim 13 wherein the discoidin domain receptor tyrosine kinase protein is a discoidin domain receptor 1, or a part thereof, and the collagen is a type I, II, III, IV, or V collagen, or a part thereof.

16. A method as claimed in claim 13 wherein the discoidin domain receptor tyrosine kinase protein is a discoidin domain receptor 2, or a part thereof, and the collagen is a type I or III collagen, or a part thereof.

17. A method as claimed in claim 13 wherein the test substance is a carbohydrate moiety of a collagen, or a mimetic thereof, or a peptide derived from the domain of a DDR that binds to a collagen.

18. A method for treating or preventing a condition involving a discoidin domain receptor tyrosine kinase-mediated signaling pathway, which method comprises administering to a patient in need thereof an amount of a substance which is effective to interfere with the signaling pathway wherein the substance is (a) a discoidin domain receptor tyrosine kinase or part thereof; (b) a collagen or part thereof; (c) a substance first identified by a method as claimed in claim 13, (c) an isolated complex as claimed in claim 1; (d) a peptide as claimed in claim 6; (e) a molecule as claimed in claim 7, or (f) an antibody as claimed in claim 8

19. A composition which comprises (a) an isolated and purified discoidin domain receptor tyrosine kinase or part thereof; b) a collagen or part thereof; (c) a substance fit identified by a method as claimed in claim 13, (c) an isolated complex as claimed in claim 1 (d) a peptide as claimed in claim 6; (e) a molecule as claimed in claim 7, or (f) an antibody as claimed in claim 8.

20. A composition as claimed in claim 19 which comprises an extracellular domain of a discoidin domain receptor tyrosine kinase, or the portion of the extracellular domain which binds to the carbohydrate moiety of a collagen, or mimetics thereof.

21. A method for up-regulating MMP-1 expression in a cell comprising administering a discoidin domain receptor 2 or an oligomer thereof.
